(19) United States
(12) Patent Application Publication
(10) Pub. No.: US 2007/0244155 A1
(43) Pub. Date: Oct. 18, 2007

(54) Bicyclic carboxylic acid derivatives useful for treating metabolic disorders

(75) Inventors: Rajiv Sharma, Fremont, CA (US); Michelle Akerman, San Francisco, CA (US); Mario G. Cardozo, San Francisco, CA (US); Jonathan B. Houze, San Mateo, CA (US); An-Rong Li, South San Francisco, CA (US); Jinquian Liu, Palo Alto, CA (US); Jiwen Liu, Foster City, CA (US); Zhihua Ma, San Mateo, CA (US); Julio C. Medina, San Carlos, CA (US); Michael J. Schmitt, Oakland, CA (US); Ying Sun, Albany, CA (US); Yingcai Wang, Fremont, CA (US); Zhongyu Wang, San Mateo, CA (US); Liusheng Zhu, Burlingame, CA (US)

Correspondence Address: AMGEN INC., MAIL STOP 28-2-C, ONE AMGEN CENTER DRIVE, THOUSAND OAKS, CA, 91320-1799, US

(73) Assignee: AMGEN INC., Thousand Oaks, CA (US)

(21) Appl. No.: 11/717,945

(22) Filed: Mar. 13, 2007

Related U.S. Application Data
(60) Provisional application No. 60/782,706, filed on Mar. 14, 2006.
(60) Provisional application No. 60/905,207, filed on Mar. 5, 2007.

(51) Int. Cl. A61K 31/4704; A61K 31/405; A61K 31/366; A61K 31/353; A61K 31/192

(52) U.S. Cl. 514/312; 514/419; 514/457; 514/450; 514/569; 546/157; 548/495; 549/285; 549/355; 514/456; 562/467; 549/405

(57) ABSTRACT

Compounds having the general formula I and/or the general formula II are useful, for example, for treating metabolic disorders in a subject 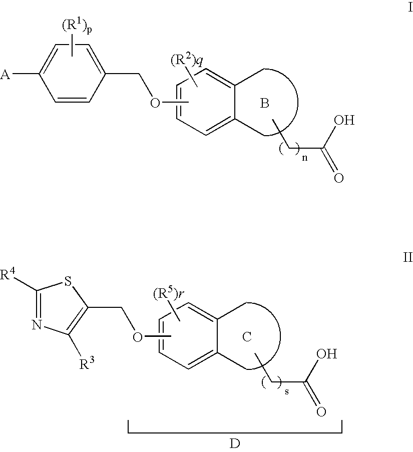 where the variables are provided herein. Compositions and methods for using the compounds for preparing medicaments and for treating metabolic disorders such as, for instance, type II diabetes are disclosed.

### CROSS REFERENCES TO RELATED APPLICATIONS

This application claims priority to U.S. Provisional Application No. 60/782,706, filed on Mar. 14, 2006, and the U.S. Provisional Application titled “Bicyclic Carboxylic Acid Derivatives Useful for Treating Metabolic Disorders” filed on Mar. 5, 2007, which are hereby incorporated by reference in their entireties and for all purposes as if fully set forth herein.

### FIELD OF THE INVENTION

The present invention relates to compounds capable of modulating the G-protein-coupled receptor GPR40, compositions comprising the compounds, and methods for their use for controlling insulin levels in vivo and for the treatment of conditions such as type II diabetes, hypertension, ketoacidosis, obesity, glucose intolerance, and hypercholesterolemia and related disorders associated with abnormally high or low plasma lipoprotein, triglyceride or glucose levels.

### BACKGROUND OF THE INVENTION

The production of insulin is central to the regulation of carbohydrate and lipid metabolism. Insulin imbalances lead to conditions such as type II diabetes mellitus, a serious metabolic disease that afflicts around 5% of the population in Western Societies and over 150 million people worldwide. Insulin is secreted from pancreatic β cells in response to elevated plasma glucose which is augmented by the presence of fatty acids. The recent recognition of the function of the G-protein coupled receptor GPR40 in modulating insulin secretion has provided insight into regulation of carbohydrate and lipid metabolism in vertebrates, and further provided targets for the development of therapeutic agents for disorders such as obesity, diabetes, cardiovascular disease and dyslipidemia.

GPR40 is a member of the gene superfamily of G-protein coupled receptors (“GPCRs”). GPCRs are membrane proteins characterized as having seven putative transmembrane domains that respond to a variety of molecules by activating intra-cellular signaling pathways critical to a diversity of physiological functions. GPR40 was first identified as an orphan receptor (i.e., a receptor without a known ligand) from a human genomic DNA fragment. Sawzdargo et al., Biochem. Biophys. Res. Commun., 239:543-547 (1997). GPR40 is highly expressed in pancreatic β cells and insulin-secreting cell lines. GPR40 activation is linked to modulation of the G_{q} family of intra-cellular signaling proteins and concomitant induction of elevated calcium levels. It has been recognized that fatty acids serve as ligands for GPR40, and that fatty acids regulate insulin secretion through GPR40. Itoh et al., Nature, 422:173-176 (2003); Briscoe et al., J. Biol. Chem., 278:11303-11311 (2003); Kotarsky et al., Biochem. Biophys. Res. Commun., 301:406-410 (2003).

The prevalence of type II diabetes, obesity, hypertension, cardiovascular disease and dyslipidemia underscores the need for new therapies to effectively treat or prevent these conditions.

### SUMMARY OF THE INVENTION

Provided herein are compounds, pharmaceutical compositions, and methods useful for treating or preventing a condition or disorder such as type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer, and edema. Also provided is the use of compounds of the invention for treating such conditions or disorders and the use of the compounds in the manufacture of medicaments for treating such conditions or disorders.

In one aspect, the invention provides compounds of formula I 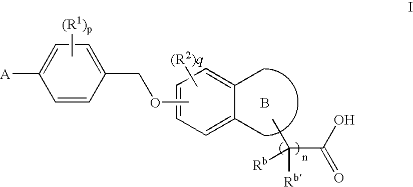
and pharmaceutically acceptable salts, esters, solvates, tautomers, stereoisomers, and/or prodrugs thereof,
wherein,
A is selected from an aryl group or a heterocyclyl group;
B is a 5 to 7 membered carbocyclic or heterocyclic ring;
R¹ is selected from halo, cyano, C₁-C₆ alkyl, —OH, or C₁-C₆ alkoxy;
R² is selected from halo, C₁-C₆ alkyl, —OH, or C₁-C₆ alkoxy;
n is selected from 0, 1, or 2;
p is selected from 0, 1, or 2;
q is selected from 0, 1, or 2;
each R¹ is independently selected if p is 2;
each R² is independently selected if q is 2; and
R^{b} and R^{b}′ are independently selected from —H, and halo.
   In such embodiments, each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl.
   The B ring may further be substituted with an oxo group (═O) or may include a group of formula ═CR^{a}R^{a′} where R^{a} and R^{a′} are independently selected from H or C₁-C₄ alkyl groups. In some embodiments, B does not include an O atom if B is a 5-membered ring that comprises four C atoms.

In some embodiments of the compounds of formula I, R^{b} and R^{b′} are independently selected from H and F. In some such embodiments, n is 1 and R^{b} and R^{b′} are either both H or are both F. In some embodiments, both R^{b} and R^{b′} are H.

In some embodiments of the compounds of formula I, n is 1.

In some embodiments of the compounds of formula I, p is 0.

In some embodiments of the compounds of formula I, q is 0.

In some embodiments of the compounds of formula I, A is an optionally substituted aryl group. In some such embodiments, A is an unsubstituted phenyl group or is a phenyl group that is substituted with at least one cyano, —CF₃, C₁-C₆ alkyl, —OH, or C₁-C₆ alkoxy group. In other such embodiments A is a phenyl group substituted with at least one methyl group, methoxy group, ethoxy group, propoxy group, butoxy group, or pentoxy group.

In some embodiments of the compounds of formula I, B is a 5 or 6 membered carbocyclic or heterocyclic ring. In some such embodiments, B is a 5 or 6 membered carbocyclic ring.

In some embodiments of the compounds of formula I, the compound has a formula selected from:

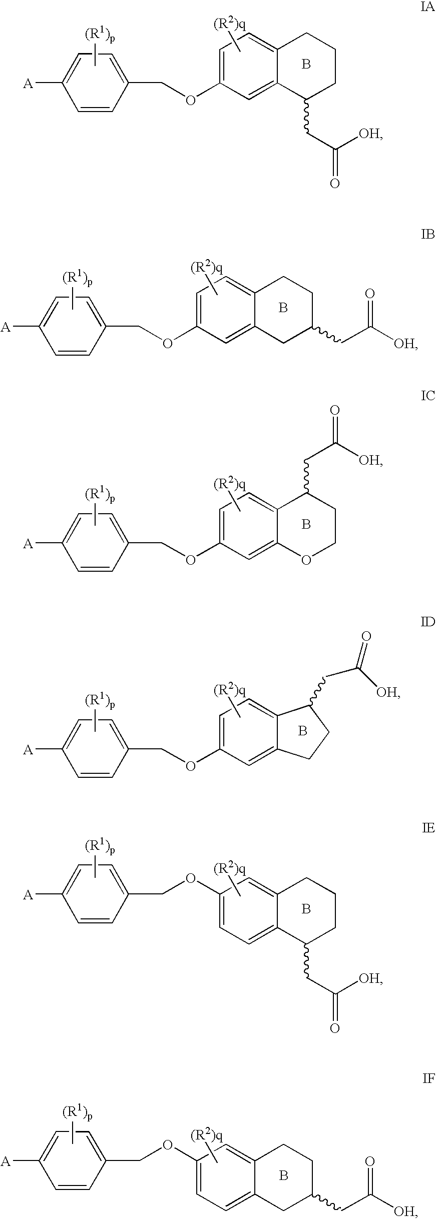

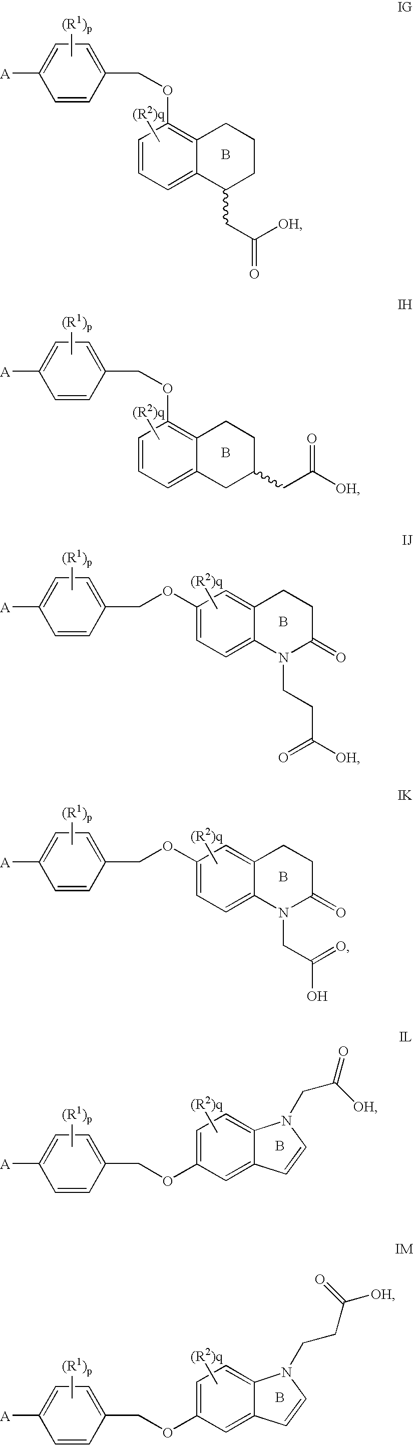

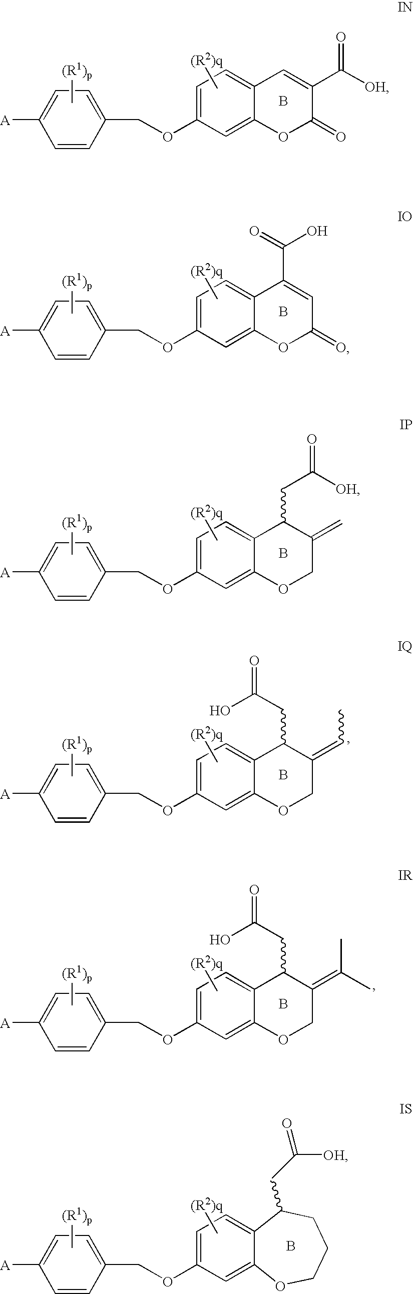

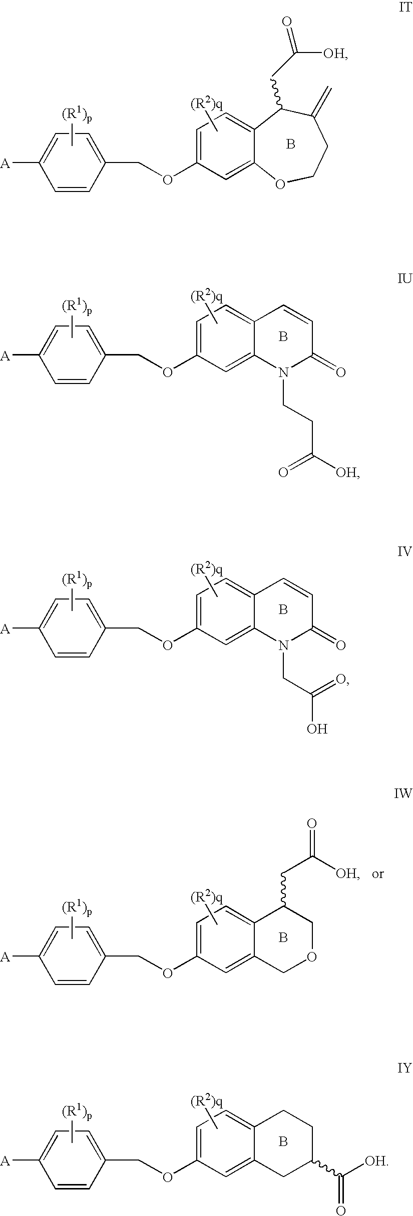

In such embodiments, the B ring may be further substituted with a halo, a C₁-C₆ alkyl group, an oxo, a C₂-C₆ alkenyl group, or a group of formula ═CR^{a}R^{a′} where R^{a} and R^{a′} are independently selected from H or C₁-C₄ alkyl groups. In the above structures, a wavy bond indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers. In some embodiments, the compound has the formula of any one or more of the structures shown above.

In another aspect, the invention provides compounds of formula II 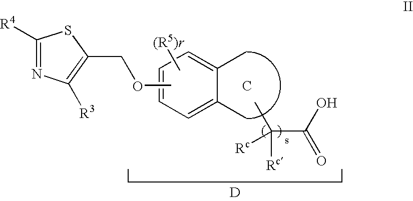
and pharmaceutically acceptable salts, esters, solvates, tautomers, stereoisomers, and/or prodrugs thereof,
wherein,
C is a 5 to 7 membered carbocyclic or heterocyclic ring;
D is a fragment of the compound as shown above;
R³ is selected from —H, halo, or C₁-C₆ alkyl;
R⁴ is an aryl group;
R⁵ is selected from halo, C₁-C₆ alkyl, —OH, or C₁-C₆ alkoxy;
s is selected from 0, 1, or 2;
r is selected from 0, 1, or 2;
each R⁵ is independently selected if r is 2; and
R^{c} and R^{c′} are independently selected from —H and halo.
   In such embodiments, each of the above alkyl and aryl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl.
   The C ring may further be substituted with an oxo group (═O) or may include a group of formula ═CR^{a}R^{a′} where R^{a} and R^{a′} are independently selected from H or C₁-C₄ alkyl groups.

In some embodiments of the compounds of formula II, R^{c} and R^{c′} are independently selected from H and F. In some such embodiments, s is I and R^{c} and R^{c′} are either both H or are both F. In some embodiments, both R^{c} and R^{c′} are H.

In some embodiments of the compounds of formula II, s is 1.

In some embodiments of the compounds of formula II, r is 0.

In some embodiments of the compounds of formula II, R⁴ is an unsubstituted phenyl group or is a phenyl group that is substituted with at least one cyano, halo, —CF₃, C₁-C₆ alkyl, —OH, or C₁-C₆ alkoxy group. In some such embodiments, R⁴ is a phenyl group substituted with a methyl group. In some such embodiments, R⁴ is a phenyl group substituted in the para position with a methyl group

In some embodiments of the compounds of formula II, R³ is a C₁-C₆ alkyl group. In some such embodiments, R³ is a methyl, ethyl, or propyl group. In some of these embodiments, R³ is a methyl group.

In some embodiments of the compounds of formula II, C is a 5 or 6 membered carbocyclic or heterocyclic ring. In some such embodiments, C is a 5 or 6 membered carbocyclic ring.

In some embodiments of the compounds of formula II, the fragment D has a formula selected from: 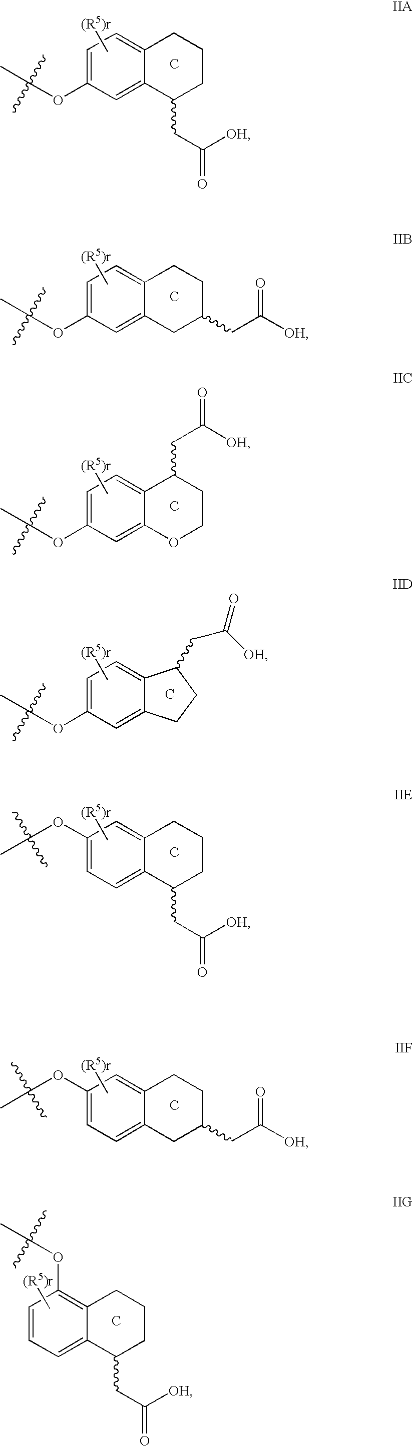 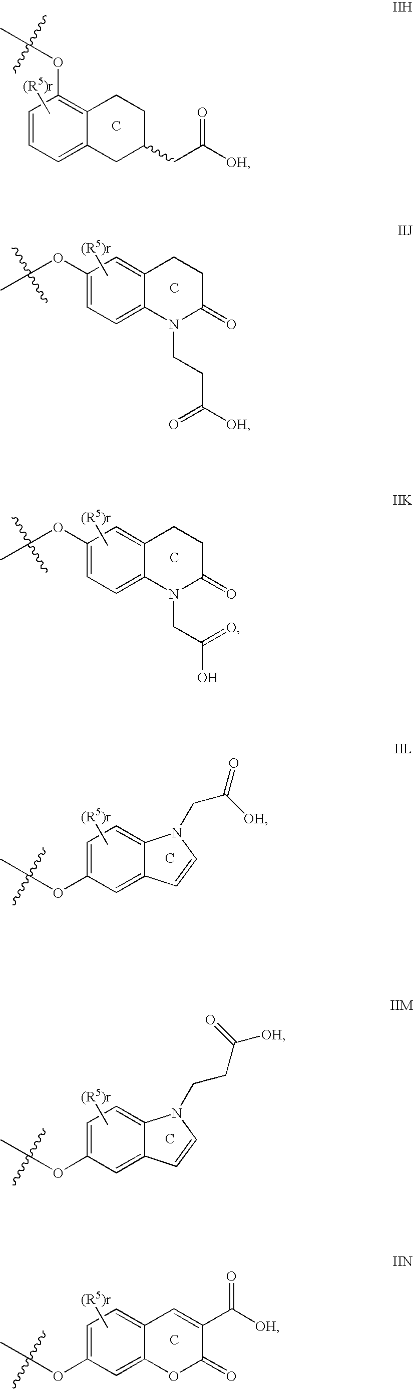 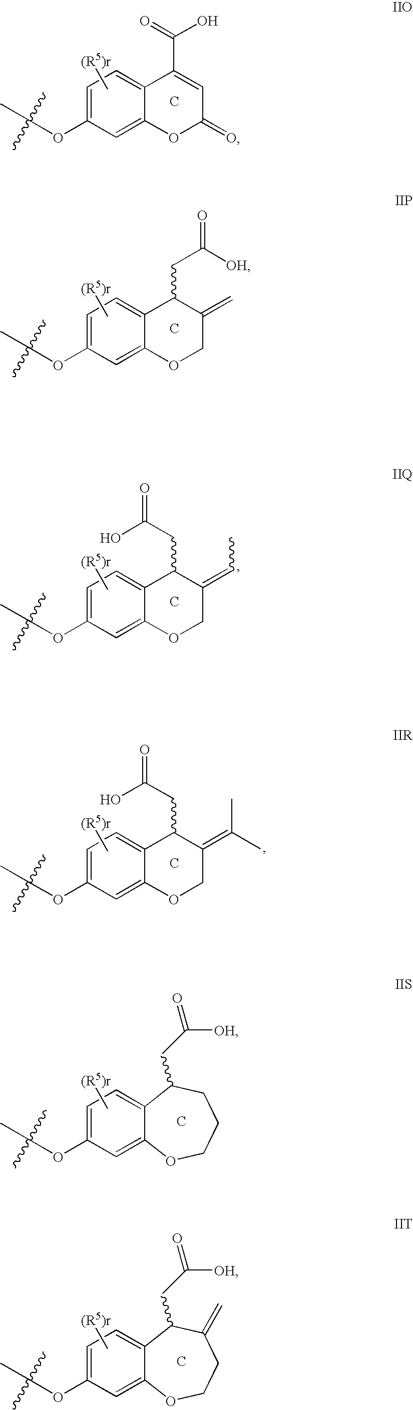 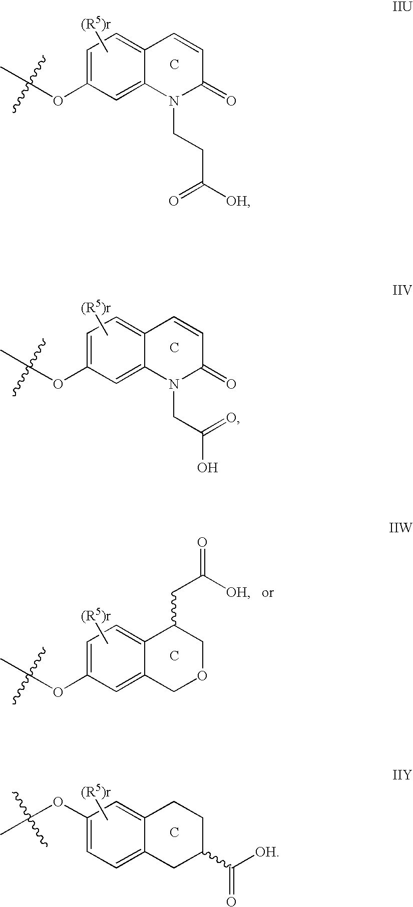
In such embodiments, the C ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups. In the structures shown above, a wavy bond indicates a point of attachment when drawn across a bond, indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers. In some embodiments, the compound has the formula of any one or more of the structures shown above.

In another aspect, the invention provides compounds of formula III
F-L₁-E-L₂-L₃-G III
and pharmaceutically acceptable salts, esters, solvates, tautomers, stereoisomers, and/or prodrugs thereof,
wherein,
E is selected from an aryl group or a heterocyclyl group;
F is selected from —H, an aryl group, or a heterocyclyl group;
L₁ is selected from a bond, —O—, —NH—, —S—, —CH₂—, —C(═O)—, —SO—, or —SO₂—;
L₂ is selected from —CH₂)ₘ—, or O—(CH₂)ₘ— where m is selected from 1 or 2;
L₃ is —O—, —NH—, —S—, or L₂ and L₃, when taken together, represent a group of formula —CH═CH—, or —C(═CH₂)—; and
G is selected from 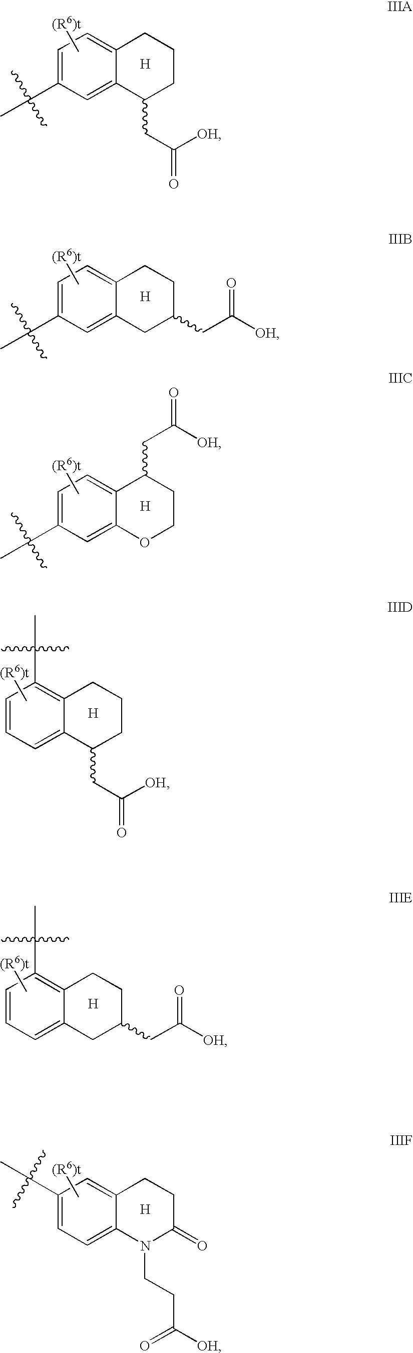 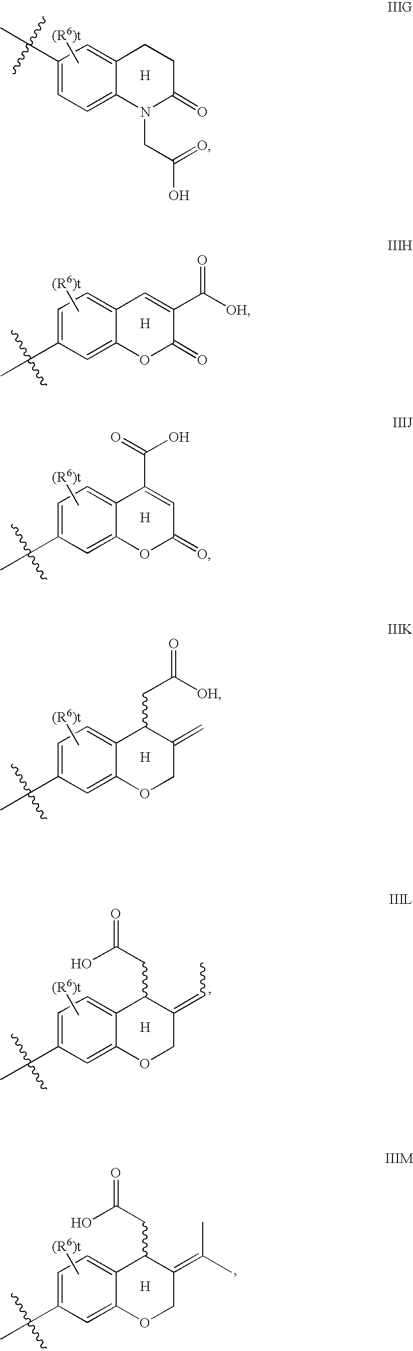 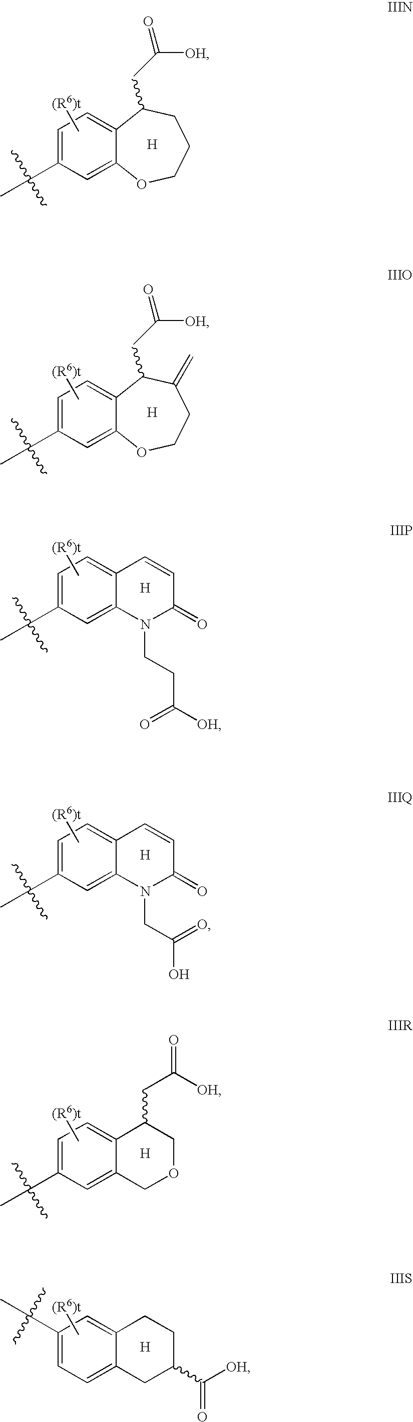 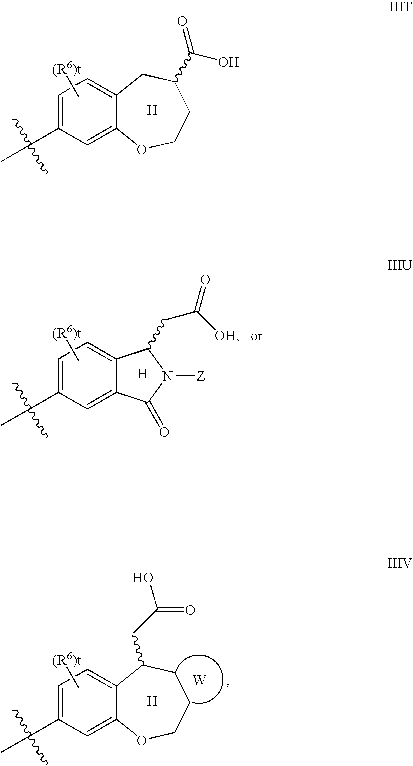
   wherein,
R⁶ is selected from halo, C₁-C₆ alkyl, —OH, or C₁-C₆ alkoxy;
t is selected from 0, 1, or 2;
each R⁶ is independently selected if t is 2;
Z is selected from H and C₁-C₆ alkyl; and
W is a 5-7 membered heterocyclic ring.
   The H ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups, and a wavy bond indicates a point of attachment when drawn across a bond, indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers. If G is IIIT, L³ is —O—, L² is —CH₂)—, L¹ is a bond, E is an unsubstituted benzene ring, and F and L² are oriented in a meta substitution pattern on E, then F is not substituted with two methyl groups. If G is IIIT, L³ is —O—, L² is —CH₂)—, L¹ is —O—, E is an unsubstituted benzene ring, and L¹ and L² are oriented in a meta substitution pattern on E, then F is not an unsubstituted benzene ring. Each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl.

In some embodiments of the compounds of formula III, L₁ is a bond or —O—. In some such embodiments, L₁ is a bond. In other such embodiments, L₁ is —O—.

In some embodiments of the compounds of formula III, L₃ is —O—, or L₂ and L₃, when taken together, represent a group of formula —CH═CH—, or —C(═CH₂)—. In some such embodiments, L₃ is —O—.

In some embodiments of the compounds of formula III, L₂ is —CH₂)ₘ— and m is 1.

In some embodiments of the compounds of formula III, E is an optionally substituted thiazole group. In some such embodiments, the compound has the formula IV where R⁷ is selected from —H, halo, or C₁-C₆ alkyl and the other variables have any of the definitions of the other embodiments 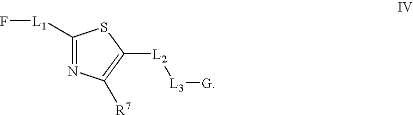
In some such embodiments, R⁷ is a C₁-C₆ alkyl groups such as a methyl group.

In some embodiments of the compounds of formula III, E is an optionally substituted phenyl group. In some such embodiments, the compound has the formula VA or VB where R⁸ is selected from halo, cyano, C₁-C₆ alkyl, —OH, or C₁-C₆ alkoxy; u is selected from 0, 1, or 2; each R⁸ is independently selected if u is 2, and the other variables have any of the values of the other embodiments

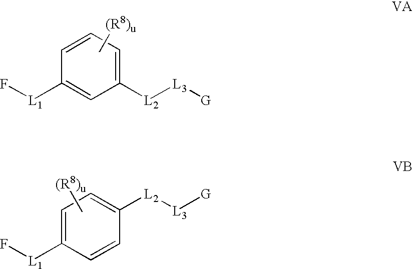

In some embodiments of the compounds of formula III, F is an unsubstituted phenyl group or is a phenyl group that is substituted with at least one cyano, —CF₃, C₁-C₆ alkyl, —OH, or C₁-C₆ alkoxy group. In some such embodiments, F is a phenyl group substituted with at least one methyl group, methoxy group, ethoxy group, propoxy group, butoxy group, or pentoxy group.

In some embodiments of the compounds of formula III, G is selected from one of IIIA-IIIS. In other embodiments, G is selected from one of IIIT, IIIU, or IIIV. In some embodiments where G is IIIU, X is H whereas in other such embodiments, Z is methyl.

In some embodiments of the compounds of formula III where G is IIIV, W is a heteroaryl ring. In some such embodiments, W is an isoxazole. In some such embodiments, IIIV, has the formula IIIV′.

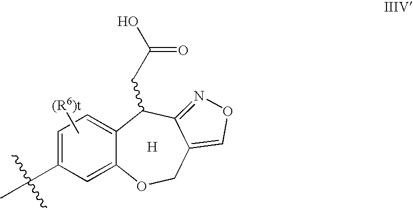

In another aspect, the invention provides compounds of formula VI 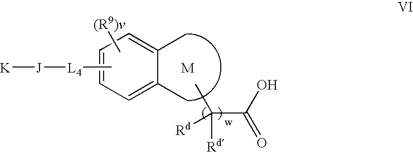
and pharmaceutically acceptable salts, esters, solvates, tautomers, stereoisomers, and/or prodrugs thereof,
wherein,
J is selected from an aryl group or a heterocyclyl group;
K is selected from —H, —CF₃, halo, cyano, C₁-C₆ alkyl, —OH, C₁-C₆ alkoxy, —O-aryl, an aryl group, or a heterocyclyl group;
M is a 5 to 7 membered carbocyclic or heterocyclic ring;
L₄ is selected from —H₂CH₂—, —CH═CH—, or —C(═CH₂)—;
R⁹ is selected from halo, C₁-C₆ alkyl, —OH, or C₁-C₆ alkoxy;
v is selected from 0, 1, or 2;
w is selected from 0, 1, or 2;
each R⁹ is independently selected if v is 2; and
R^{d} and R^{d′} are independently selected from —H and halo,
   and further wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl,
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
and further wherein the M ring may be further substituted with an oxo group or a group of formula ═CR^{a}R^{a′} where R^{a} and R^{a′} are independently selected from H or C₁-C₄ alkyl groups.

In some embodiments of the compounds of formula VI, R^{d} and R^{d′} are independently selected from H and F. In some such embodiments, w is 1 and R^{d} and R^{d′} are either both H or are both F. In some embodiments, both R^{d} and R^{d′} are H.

In some embodiments of the compounds of formula VI, w is 1.

In some embodiments of the compounds of formula VI, v is 0.

In some embodiments of the compounds of formula VI, J is an optionally substituted aryl group.

In some embodiments of the compounds of formula VI, J is an optionally substituted thiazole group.

In some embodiments of the compounds of formula VI, M is a 6 membered carbocyclic or heterocyclic ring. In some such embodiments, M is a 6 membered carbocyclic ring.

In some embodiments of the compounds of formula I, II, III, and/or VI, the B ring, the C ring, the H ring, or the M ring is substituted with a ═CR^{a}R^{a′} group where R^{a} and R^{a′} are independently selected from H and C₁-C₄ alkyl groups.

In another aspect, the invention provides compounds of formula VII 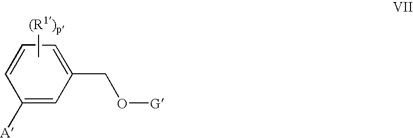
or a pharmaceutically acceptable salt, ester, solvate, tautomer, stereoisomer, or prodrug thereof,
wherein,
A′ is selected from an aryl group or a heterocyclyl group;
R^{1′} is selected from halo, cyano, C₁-C₆ alkyl, —OH, or C₁-C₆ alkoxy;
p′ is selected from 0, 1, or 2;
each R^{1′} is independently selected if p is 2; and
G′ is selected from 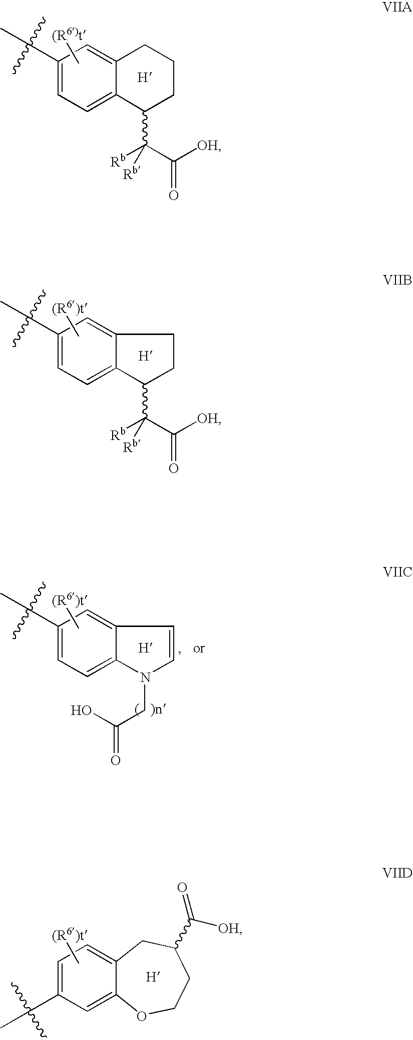
   wherein,
R^{6′} is selected from halo, C₁-C₆ alkyl, —OH, or C₁-C₆ alkoxy;
t′ is selected from 0, 1, or 2;
each R^{6′} is independently selected if t′ is 2;
R^{b} and R^{b′} are independently selected from —H and halo; and
n′ is selected from 1 or 2
   and further wherein the H′ ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups, and a wavy bond indicates a point of attachment when drawn across a bond, or indicates the R and S enantiomers individually or as a mixture of the R and S enantiomers;
   and further wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl,
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
   and further wherein, A′ does not have the following formula

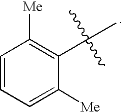

In some embodiments of the compounds of formula VII, A′ is a phenyl group that is substituted with at least one cyano, —CF₃, C₁-C₆ alkyl, —OH, or C₁-C₆ alkoxy group.

In some embodiments of the compounds of formula VII, A′ is a phenyl group that is substituted with at least one —CF₃, —F, —Cl, —Br, —I, methoxy group, ethoxy group, propoxy group, butoxy group, or pentoxy group.

In some embodiments of the compounds of formula VII, p′ is 0.

In some embodiments of the compounds of formula VII, t′ is 0.

In some embodiments of the compounds of formula VII, G′ is VIIA. In other embodiments, G′ is VIIB. In still other embodiments, G′ is VIIC. In still other embodiments, G′ is VIID.

In some embodiments of the compounds of formula VII, H′ is not further substituted.

In some embodiments of the compounds of formula VII, H′ is substituted with a C₁-C₄ alkyl group.

In some embodiments of the compounds of formula VII, H′ is substituted with a group of formula ═CR^{a}R^{a′} where R^{a} and R^{a′} are independently selected from H or C₁-C₄ alkyl groups.

In other aspects, the invention provides pharmaceutical compositions that include a pharmaceutically acceptable carrier, diluent or excipient and any of the compounds, or pharmaceutically acceptable salts, esters, solvates, tautomers, stereoisomers, and/or prodrugs thereof of any of the embodiments described herein. In other aspects the invention thus also provides the use of any of the compounds, or pharmaceutically acceptable salts, esters, solvates, tautomers, stereoisomers, and/or prodrugs thereof of the invention in the preparation of a medicament. Such medicaments may be used in accordance with the methods described herein.

In yet other aspects, the invention provides a method for treating a disease or condition such as one of these selected from type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer or edema. Such methods include administering to a subject in need thereof a therapeutically effective amount of any of the compounds, or pharmaceutically acceptable salts, esters, solvates, tautomers, stereoisomers, and/or prodrugs thereof or pharmaceutical compositions of any of the embodiments described herein. In some embodiments, the disease or condition is type II diabetes.

In still another aspect, the invention provides a method for treating a disease or condition responsive to the modulation of GPR40. Such methods include administering to a subject in need thereof a therapeutically effective amount of any of the compounds, or pharmaceutically acceptable salts, esters, solvates, tautomers, stereoisomers, and/or prodrugs thereof or pharmaceutical compositions of any of the embodiments described herein. In some embodiments, the disease or condition is selected from type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer, or edema. In some such embodiments, the disease or condition is type II diabetes.

In some embodiments of the methods described herein, the compounds, or pharmaceutically acceptable salts, esters, solvates, tautomers, stereoisomers, and/or prodrugs thereof or pharmaceutical compositions may be administered orally, parenterally or topically. In some such embodiments, the compound, pharmaceutically acceptable salt, ester, solvate, tautomer, stereoisomer, or prodrug or pharmaceutical composition is administered in combination with a second therapeutic agent. The second therapeutic agent may be administered before during or after the compound, pharmaceutically acceptable salt, ester, solvate, tautomer, stereoisomer, or prodrug is administered. In some embodiments, the second therapeutic agent is a metformin or a thiazolidinedione.

In another aspect, the invention also provides a method for modulating GPR40 function in a cell. Such methods include contacting the cell with the compound, pharmaceutically acceptable salt, ester, solvate, tautomer, stereoisomer, or prodrug or the pharmaceutical composition of any of the embodiments described herein.

In yet another aspect, the invention provides a method for modulating GPR40 function. Such methods include contacting GPR40 with the compound, pharmaceutically acceptable salt, ester, solvate, tautomer, stereoisomer, or prodrug or the pharmaceutical composition of any of the embodiments described herein.

In still another aspect, the invention provides a method for modulating circulating insulin concentration in a subject. Such methods include administering to the subject the compound, pharmaceutically acceptable salt, ester, solvate, tautomer, stereoisomer, or prodrug or the pharmaceutical composition of any of the embodiments described herein. In some embodiments the insulin concentration is increased after administration whereas in other embodiments the insulin concentration is decreased after administration.

In still another aspect, the invention provides the use of the compound, pharmaceutically acceptable salt, ester, solvate, tautomer, stereoisomer, or prodrug or the pharmaceutical composition of any of the embodiments described herein in the manufacture of a medicament for: treating a disease or condition selected from type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer or edema; treating a disease or condition responsive to the modulation of GPR40; modulating GPR40 function in a cell; modulating GPR40 function; and/or modulating circulating insulating concentration in a subject.

In still further aspects, the invention provides any of the Example compounds described herein individually or as a member of a group that includes any number of or all of the other Example compounds.

Other objects, features and advantages of the invention will become apparent to those skilled in the art from the following description and claims.

### DETAILED DESCRIPTION OF THE INVENTION

The invention provides GPR40 modulating compounds. Such compounds may be used to prepare pharmaceutical compositions and are useful in various methods of treating and/or preventing a variety of conditions and disorders such as type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer, and edema.

The terms “treat”, “treating” and “treatment”, as used herein, are meant to include alleviating or abrogating a condition or disease and/or its attendant symptoms and alleviating. The terms “prevent”, “preventing” and “prevention”, as used herein, refer to a method of delaying or precluding the onset of a condition or disease and/or its attendant symptoms, barring a subject from acquiring a condition or disease or reducing a subject's risk of acquiring a condition or disease.

The term “therapeutically effective amount” refers to that amount of the compound that will elicit the biological or medical response of a tissue, system, or subject that is being sought. The term “therapeutically effective amount” includes that amount of a compound that, when administered, is sufficient to prevent development of, or alleviate to some extent, one or more of the symptoms of the condition or disorder being treated in a subject. The therapeutically effective amount in a subject will vary depending on the compound, the disease and its severity and the age, weight, etc., of the subject to be treated.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “modulate”, “modulation” and the like refer to the ability of a compound to increase or decrease the function or activity of GPR40 either directly or indirectly. Inhibitors are compounds that, for example, bind to, partially or totally block stimulation, decrease, prevent, delay activation, inactivate, desensitize, or down regulate signal transduction, such as, for instance, antagonists. Activators are compounds that, for example, bind to, stimulate, increase, activate, facilitate, enhance activation, sensitize or up regulate signal transduction, such as agonists for instance. Modulation may occur in vitro or in vivo.

As used herein, the term “GPR40-mediated condition or disorder” and the like refer to a condition or disorder characterized by inappropriate, for example, less than or greater than normal, GPR40 activity. A GPR40-mediated condition or disorder may be completely or partially mediated by inappropriate GPR40 activity. However, a GPR40-mediated condition or disorder is one in which modulation of GPR40 results in some effect on the underlying condition or disease (e.g., a GPR40 modulator results in some improvement in patient well-being in at least some patients). Exemplary GPR40-mediated conditions and disorders include cancer and metabolic disorders, e.g., diabetes, type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, ketoacidosis, hypoglycemia, thrombotic disorders, metabolic syndrome, syndrome X and related disorders, e.g., cardiovascular disease, atherosclerosis, kidney disease, nephropathy, diabetic neuropathy, diabetic retinopathy, sexual dysfunction, dermatopathy, dyspepsia and edema.

The terms “comprising”, “comprises”, and “including” are used herein in their open, non-limiting sense. For example, a composition comprising component “A” includes component “A”, but may also include other components.

As used herein, unless otherwise specified, the term “alkyl” means a saturated straight chain or branched non-cyclic hydrocarbon having from 1 to 20 carbon atoms, preferably 1-10 carbon atoms and most preferably 1-4 carbon atoms. Representative saturated straight chain alkyls include, but are not limited to, -methyl, -ethyl, -n-propyl, -n-butyl, -n-pentyl, -n-hexyl, -n-heptyl, -n-octyl, -n-nonyl and -n-decyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted.

As used herein, unless otherwise specified, the term “alkenyl” means an unsaturated straight chain or branched non-cyclic hydrocarbon having from 2 to 20 carbon atoms and at least one carbon-carbon double bond. Preferably an alkenyl has 2 to 10 carbon atoms and most preferably has 2 to 4 carbon atoms. Exemplary straight chain alkenyls include, but are not limited to, -but-3-ene, -hex-4-ene, and -oct-1-ene. Exemplary branched chain alkenyls include, but are not limited to, -2-methyl-but-2-ene, -1-methyl-hex-4-ene, and -4-ethyl-oct-1-ene. An alkenyl group can be substituted or unsubstituted.

As used herein, and unless otherwise specified, the term “alkynyl” means an alkyl group in which one or more carbon-carbon single bonds is replaced with an equivalent number of carbon-carbon triple bonds. An alkynyl group must comprise at least two carbon atoms, and can be substituted or unsubstituted. Alkynyl groups typically include from 2 to 8 carbon atoms. In some embodiments, alkynyl groups include from 2 to 6, from 2 to 4, or from 2 to 3 carbon atoms. Alkynyl groups can be substituted or unsubstituted.

The term “halo” refers to a halogen atom such as a —F, —Cl, —Br, or —I atom.

As used herein, unless otherwise specified, the term “haloalkyl” means an alkyl group in which one or more hydrogens has been replaced by a halogen atom. A halogen atom is a fluorine, chlorine, bromine, or iodine atom. In some embodiments, a haloalkyl group is a perfluoroalkyl group such as a —CF₃ group otherwise known as a trifluoromethyl group.

The term “hydroxyl” refers to the —OH substituent.

As used herein, unless otherwise specified, the term “hydroxyalkyl” means an alkyl group in which one or more hydrogens has been replaced with a hydroxyl group.

The term “alkoxy” means a structure of formula —O-alkyl where alkyl has the meaning set forth above. Representative examples of alkoxy groups include methoxy, ethoxy, propoxy, butoxy, and pentoxy groups, and the like. The term “aryloxy” means a structure of formula —O-aryl where aryl has the meaning set forth above.

The term “amino” refers to the —NH₂ group. The terms “alkylamino” and “dialkylamino” mean an amino group where one (alkylamino) or both (dialkylamino) of the hydrogen atoms is replaced with an alkyl group. Thus, the terms “alkylamino” and “dialkylamino” have a structure of formula —NH-alkyl and —N(alkyl)alkyl, respectively where alkyl has the meaning set forth above.

The phrase “carbocyclic ring” means a ring system in which each of the ring members is a carbon atom. Examples of carbocyclic rings include cyclopentane, cyclohexane, cycloheptane, cyclooctane, cyclopentene, cyclohexene, cycloheptene, cyclohexadiene, and benzene. Carbocyclic rings may be substituted or unsubstituted and may be saturated or include unsaturation. Carbocyclic rings may be aromatic or non-aromatic and, in some embodiments, include from 3 to 14 or 3 to 8 ring members, but may include more. In some embodiments, carbocyclic rings include 5 to 7 ring members and, in some embodiments, may include 6 ring members. In some embodiments, carbocyclic rings may be non-aromatic. Carbocyclic rings can be substituted or unsubstituted.

The phrase “heterocyclic ring” means a ring system in which one or more ring members is a heteroatom such as a N, O, or S atom. Heterocyclic rings may be substituted or unsubstituted and may be saturated or include unsaturatation. Heterocyclic rings may be aromatic or non-aromatic and, in some embodiments, include from 3 to 14 ring members, but may include more. In some embodiments, heterocyclic rings include 5 to 8 ring members, include 5 to 7 ring members and, in some embodiments, may include 6 ring members. In some embodiments, heterocyclic rings may be non-aromatic. Heterocyclic rings can be substituted or unsubstituted. Some heterocyclic rings include 1 heteroatom whereas other heterocyclic rings include 2, 3, or more heteroatoms.

As used herein, unless otherwise specified, the term “aryl” means a carbocyclic ring or ring system in which at least one ring is aromatic. In some embodiments, aryl groups have from 6 to 14 ring members. In other embodiments, aryl groups have from 6 to 12 or from 6 to 10 ring members. The ring atoms of a carbocyclic aryl group are all carbon atoms. Aryl groups include mono-, bi-, and tricyclic groups as well as benzo-fused carbocyclic moieties such as, but not limited to, 5,6,7,8-tetrahydronaphthyl and the like. In some embodiments, the aryl group is a monocyclic ring or is a bicyclic ring. Representative aryl groups include, but are not limited to, phenyl, tolyl, anthracenyl, biphenyl, fluorenyl, indenyl, azulenyl, phenanthrenyl and naphthyl. An aryl group can be unsubstituted or substituted.

The term “heteroaryl” means an aryl group in which one or more, but not all, of the ring carbon atoms is substituted by a heteroatom. Exemplary heteroatoms are N, O, and S. In some embodiments, heteroaryl groups have from 5 to 14 ring members. In other embodiments, heteroaryl groups have from 5 to 10, from 5 to 8, or from 5 to 7 ring members. Representative examples of heteroaryl groups include, but are not limited to, 1-pyrrolyl, 2-pyrrolyl, 3-pyrrolyl, 1-pyrazolyl, 3-pyrazolyl, 5-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, dibenzofuryl, 2-thienyl, 3-thienyl, 2-pyridyl, 3-pyridyl, 4-pyridyl, 2-pyrimidyl, 4-pyrimidyl, 2-pyrimidinyl, 4-pyrimidinyl, 5-pyrimidinyl, 3-pyridazinyl, 4- pyridazinyl, 5-benzothiazolyl, 2-benzoxazolyl, 5-benzoxazolyl, benzo[c][1,2,5]oxadiazolyl, purinyl, 2-benzimidazolyl, 5-indolyl, 1H-indazolyl, 1-isoquinolyl, 5-isoquinolyl, 2-quinoxalinyl, 5-quinoxalinyl, 2-quinolyl, 3-quinolyl, 4-quinolyl, 5-quinolyl, 6-quinolyl, 7-quinolyl and 8-quinolyl. A heteroaryl group can be unsubstituted or substituted.

The term “cycloalkyl” means a saturated hydrocarbon that forms at least one ring, having from 3 to 20 ring carbon atoms, and in some embodiments, from 3 to 10, from 3 to 8, or from 5 to 7 ring carbon atoms. The rings in a cycloalkyl group are not aromatic. A cycloalkyl group can be unsubstituted or substituted.

The term “heterocyclyl” means a ring system in which one or more ring members is a heteroatom. The term “heterocyclyl” includes both heteroaromatic, saturated, and partially unsaturated heterocyclic ring systems. Exemplary heteroatoms include N, O, and S.

An “oxo” substituent means an O atom that is double bonded to a carbon atom which can be shown as ═O.

The term “nitro” refers to the —NO₂ substituent.

The term “cyano” refers to the —CN substituent.

Substituents for groups such as alkyl, cycloalkyl, heterocyclic rings, carbocyclic rings, and other groups such as alkenyl and alkynyl group may include a variety of groups such as, but not limited to, —OR′, ═O, ═NR′, ═N—OR′, —NR′R″, —SR′, —R′, halogen, —OC(O)R′, —C(O)R′, —CO₂R′, —CONR′R″, —OC(O)NR′R″, —NR″C(O)R′, —NR′—C(O)NR″R′″, —NR′—SO₂NR″R′″, —NR″CO₂R′, —NH—C(NH₂)═NH, —NR′C(NH₂)═NH, —NH—C(NH₂)═NR′, —SiR′R″R′″, —S(O)R′, —SO₂R′, —SO₂NR′R″, —NR″SO₂R, —CN and —NO₂, in a number ranging from zero to three, with those groups having zero, one or two substituents being particularly preferred. R′, R″ and R′″ each independently refer to H, unsubstituted (C₁-C₈)alkyl and heteroalkyl, unsubstituted aryl, aryl substituted with one to three halogens, unsubstituted alkyl, alkoxy or thioalkoxy groups, halo(C₁-C₄)alkyl, or aryl-(C₁-C₄)alkyl groups. When R′ and R″ are attached to the same nitrogen atom, they can be combined with the nitrogen atom to form a 5-, 6- or 7-membered ring. For example, —NR′R″ is meant to include 1-pyrrolidinyl and 4-morpholinyl.

Typically, an alkyl or heteroalkyl group will have from zero to three substituents, with those groups having two or fewer substituents being preferred in the present invention. More preferably, an alkyl or heteroalkyl radical will be unsubstituted or monosubstituted. Most preferably, an alkyl or heteroalkyl radical will be unsubstituted. From the above discussion of substituents, one of skill in the art will understand that the term “alkyl” is meant to include groups such as trihaloalkyl (e.g., —CF₃ and —CH₂CF₃).

Preferred substituents for the alkyl and heteroalkyl radicals are selected from: —OR′, ═O, —NR′R″, —SR′, halogen, —OC(O)R′, —C(O)R′, —CO₂R′, —CONR′R″, —OC(O)NR′R″, —NR″C(O)R′, —NR″CO₂R′, —NR′—SO₂NR″R′″, —S(O)R′, —SO₂R′, —SO₂NR′R″, —NR″SO₂R, —CN and —NO₂, where R′ and R″ are as defined above. Further preferred substituents are selected from: —OR′, ═O, —NR′R″, halogen, —OC(O)R′, —CO₂R′, —CONR′R″, —OC(O)NR′R″, —NR″C(O)R′, —NR″CO₂R′, —NR′—SO₂NR″R′″, —SO₂R′, —SO₂NR′R″, —NR″SO₂R, —CN and —NO₂.

Suitable substituents for the aryl and heteroaryl groups are also varied and are may include, but are not limited to, -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl.

The term “pharmaceutically acceptable salt” is meant to include a salt of the active compound which is prepared with relatively nontoxic acids or bases, depending on the particular substituents found on the compound described herein. When a compound of the invention contains relatively acidic functionalities, a base addition salt can be obtained by contacting the neutral form of such compound with a sufficient amount of the desired base, either neat or in a suitable inert solvent. Examples of pharmaceutically acceptable base addition salts include sodium, potassium, calcium, ammonium, organic amino, or magnesium salt, or a similar salt. When a compound of the invention contains relatively basic functionalities, an acid addition salt can be obtained by contacting the neutral form of such compound with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propanoic, isobutyric, maleic, malonic, benzoic, succinic, suberic, fumaric, mandelic, phthalic, benzenesulfonic, p-toluenesulfonic, citric, tartaric, methanesulfonic, and the like. Also included are salts of amino acids such as arginine and the like, and salts of organic acids like glucuronic or galacturonic acids and the like (see, for example, Berge et al. (1977) J. Pharm. Sci. 66:1-19). Certain specific compounds of the invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the invention.

In addition to salt forms, the invention provides compounds which are in a prodrug form. Prodrugs of the compounds described herein are those compounds that readily undergo chemical changes under physiological conditions to provide the compounds of the invention. Additionally, prodrugs can be converted to the compounds of the invention by chemical or biochemical methods in an ex vivo environment. For example, prodrugs can be slowly converted to the compounds of the invention when placed in a transdermal patch reservoir with a suitable enzyme or chemical reagent. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound of the invention which is administered as an ester (the “prodrug”), but then is metabolically hydrolyzed to the carboxylic acid, the active entity. Additional examples include peptidyl derivatives of a compound.

As used herein, “solvate” refers to a compound of the present invention or a salt thereof, that further includes a stoichiometric or non-stoichiometric amount of solvent bound by non-covalent intermolecular forces. Where the solvent is water, the solvate is a hydrate.

Certain compounds of the invention may exist in multiple crystalline or amorphous forms. In general, all physical forms are equivalent for the uses contemplated by the invention and are intended to be within the scope of the invention.

Certain compounds of the invention possess asymmetric carbon atoms (optical centers) or double bonds; the racemates, enantiomers, diastereomers, geometric isomers and individual isomers are all intended to be encompassed within the scope of the invention.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. It should be noted that if the stereochemistry of a structure or a portion of a structure is not indicated with, for example, bold or dashed lines, the structure or portion of the structure is to be interpreted as encompassing all stereoisomers of it. A bond drawn with a wavy line indicates the R enantiomer, the S enantiomer, or a mixture of both stereoisomers.

Various compounds of the invention contain one or more chiral centers, and can exist as racemic mixtures of enantiomers, mixtures of diastereomers or enantiomerically or optically pure compounds. This invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron, 33:2725 (1997); Eliel, E. L., Stereochemistry of Carbon Compounds (McGraw-Hill, NY, 1962); and Wilen, S. H., Tables of Resolving Agents and Optical Resolutions, p. 268 (E. L. Eliel, Ed., Univ. of Notre Dame Press, Notre Dame, Ind., 1972).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., GPR40 assay reagents, and diagnostic agents, e.g., in vivo imaging agents. All isotopic variations of the compounds of the invention, whether radioactive or not, are intended to be encompassed within the scope of the invention.

In some embodiments, the compounds of the invention modulate GPR40. Depending on the biological environment (e.g., cell type, pathological condition of the subject, etc.), these compounds can modulate, e.g., activate or inhibit, the actions of GPR40. By modulating GPR40, the compounds find use as therapeutic agents capable of regulating insulin levels in a subject. The compounds find use as therapeutic agents for modulating diseases and conditions responsive to modulation of GPR40 and/or mediated by GPR40 and/or mediated by pancreatic β cells. As noted above, examples of such diseases and conditions include diabetes, obesity, hyperglycemia, glucose intolerance, insulin resistance, cancer, hyperinsulinemia, hypercholesterolemia, hypertension, hyperlipoproteinemia, hyperlipidemia, hypertriglylceridemia, dyslipidemia, ketoacidosis, hypoglycemia, metabolic syndrome, syndrome X, cardiovascular disease, atherosclerosis, kidney disease, nephropathy, thrombotic disorders, diabetic neuropathy, diabetic retinopathy, dermatopathy, dyspepsia and edema. Additionally, the compounds are useful for the treatment and/or prevention of complications of these diseases and disorders (e.g., type II diabetes, sexual dysfunction, dyspepsia and so forth).

While the compounds of the invention are believed to exert their effects by interacting with GPR40, the mechanism of action by which the compounds act is not a limiting embodiment of the invention.

Compounds contemplated by the invention include, but are not limited to, the exemplary compounds provided herein.

In one aspect, the invention provides compounds of formula I 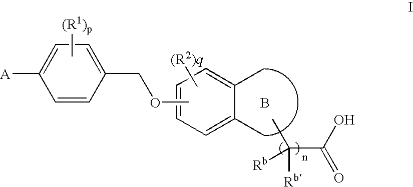
and pharmaceutically acceptable salts, esters, solvates, tautomers, stereoisomers, and/or progrugs thereof,
wherein,
A is selected from an aryl group or a heterocyclyl group;
B is a 5 to 7 membered carbocyclic or heterocyclic ring;
R¹ is selected from halo, cyano, C₁-C₆ alkyl, —OH, or C₁-C₆ alkoxy;
R² is selected from halo, C₁-C₆ alkyl, —OH, or C₁-C₆ alkoxy;
n is selected from 0, 1, or 2;
p is selected from 0, 1, or 2;
q is selected from 0, 1, or 2;
each R¹ is independently selected if p is 2;
each R² is independently selected if q is 2; and
R^{b} and R^{b}′ are independently selected from —H, or halo.
   In such embodiments, each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl.
   The B ring may further be substituted with an oxo group (═O) or may include a group of formula ═CR^{a}R^{a′} where R^{a} and R^{a′} are independently selected from H or C₁-C₄ alkyl groups. In some embodiments, B does not include an O atom if B is a 5-membered ring that comprises four C atoms.

In some embodiments of the compounds of formula I, R^{b} and R^{b′} are independently selected from H and F. In some such embodiments, n is 1 and R^{b} and R^{b′} are either both H or are both F. In some embodiments, both R^{b} and R^{b′} are H.

In some embodiments of the compounds of formula I, n is 1.

In some embodiments of the compounds of formula I, p is 0.

In some embodiments of the compounds of formula I, q is 0.

In some embodiments of the compounds of formula I, A is an optionally substituted aryl group. In some such embodiments, A is an unsubstituted phenyl group or is a phenyl group that is substituted with at least one cyano, —CF₃, C₁-C₆ alkyl, —OH, or C₁-C₆ alkoxy group. In other such embodiments A is a phenyl group substituted with at least one methyl group, methoxy group, ethoxy group, propoxy group, butoxy group, or pentoxy group.

In some embodiments of the compounds of formula I, B is a 5 or 6 membered carbocyclic or heterocyclic ring. In some such embodiments, B is a 5 or 6 membered carbocyclic ring. In other embodiments, B is heterocyclic ring that includes one heteroatom selected from N, O or S. In some embodiments, the B ring is not an aromatic ring and is at least partially saturated.

In some embodiments of the compounds of formula I, the B ring is substituted with a C₁-C₆ alkyl group such as a methyl, ethyl, propyl, or butyl group. In some such embodiments, the B ring is substituted with a methyl group. In other embodiments, the B ring does not include any further substituents.

In some embodiments of the compounds of formula I, the compound has a formula selected from: 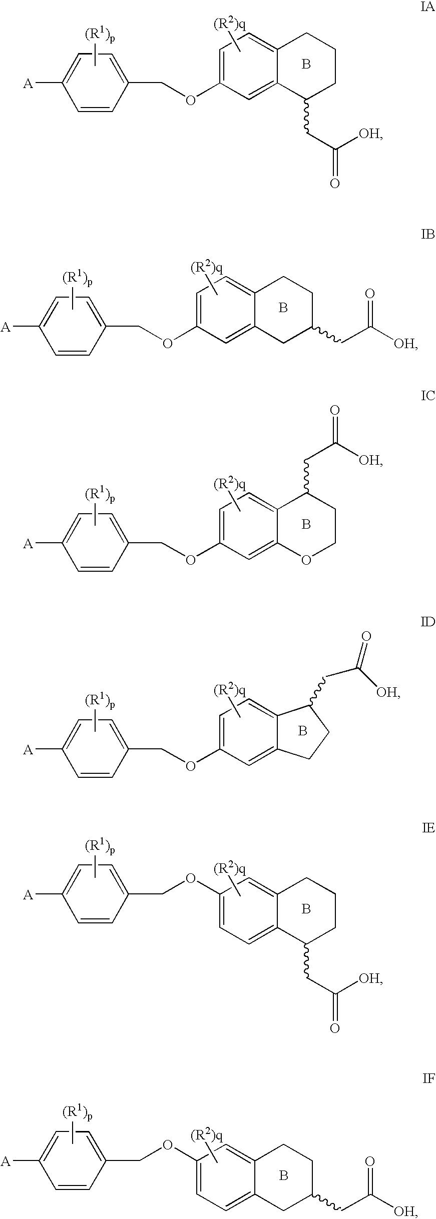 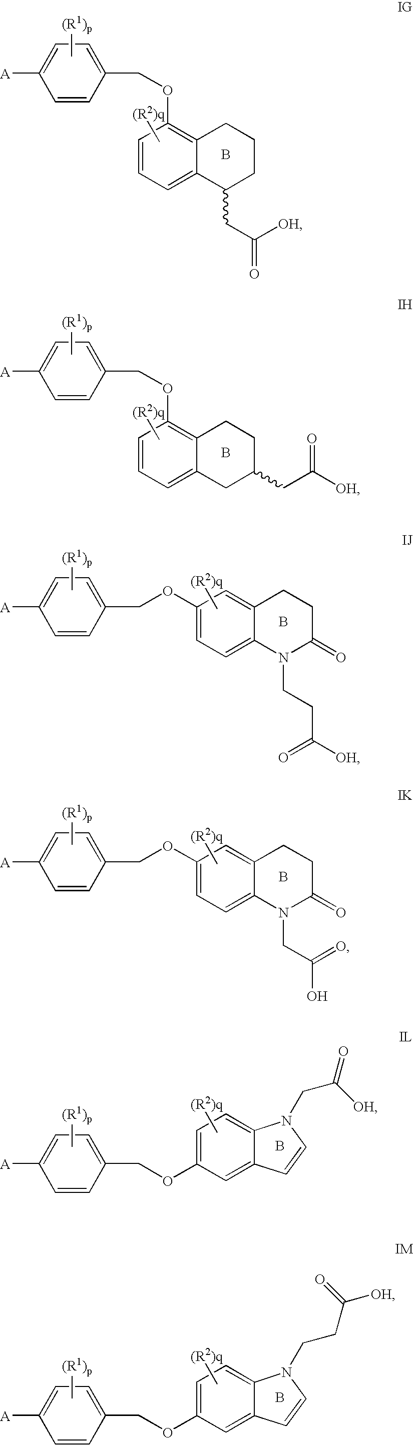 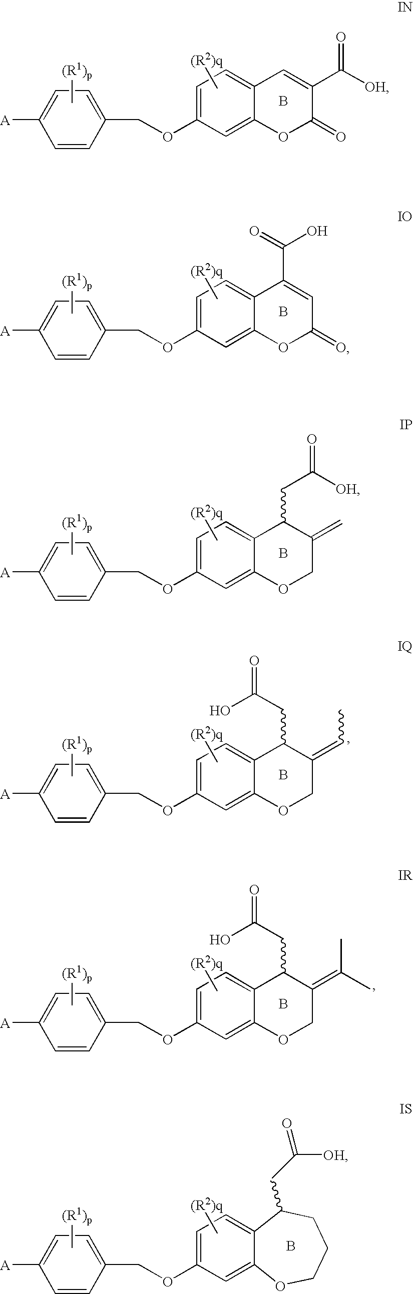 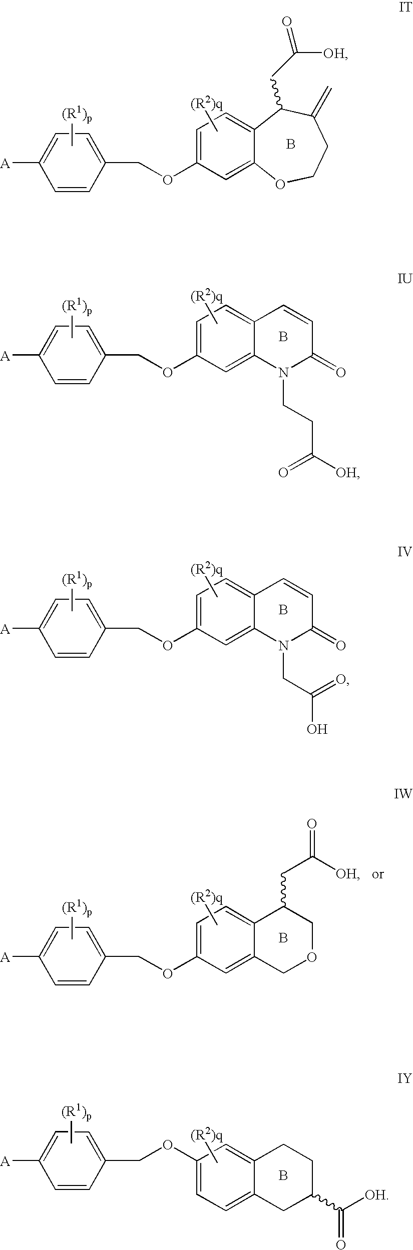
In such embodiments, the B ring may be further substituted with a halo, a C₁-C₆ alkyl group, an oxo group, a C₂-C₆ alkenyl group, or may include a group of formula ═CR^{a}R^{a′} where R^{a} and R^{a′} are independently selected from H or C₁-C₄ alkyl groups. Examples of compounds in which the B ring includes an oxo substituent include, but are not limited to, IJ, IK, and IO. Examples of compounds in which the B ring is substituted with a group of formula ═CR^{a}R^{a′} include, but are not limited to, IP, IQ, and IR. In the above structures, a wavy bond indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers. In some embodiments, the compound has the formula of any one or more of the structures shown above.

In another aspect, the invention provides compounds of formula II 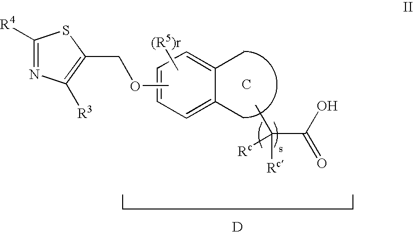
and pharmaceutically acceptable salts, esters, solvates, tautomers, stereoisomers, and/or prodrugs thereof,
wherein,
C is a 5 to 7 membered carbocyclic or heterocyclic ring;
D is a fragment of the compound as shown above;
R³ is selected from —H, halo, or C₁-C₆ alkyl;
R⁴ is an aryl group;
R⁵ is selected from halo, C₁-C₆ alkyl, —OH, or C₁-C₆ alkoxy;
s is selected from 0, 1, or 2;
r is selected from 0, 1, or 2;
each R⁵ is independently selected if r is 2; and
R^{c} and R^{c′} are independently selected from —H and halo.
   In such embodiments, each of the above alkyl and aryl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl,
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl.
   The C ring may further be substituted with an oxo group (═O) or may include a group of formula ═CR^{a}R^{a′} where R^{a} and R^{a′} are independently selected from H or C₁-C₄ alkyl groups.

In some embodiments of the compounds of formula II, R^{c} and R^{c′} are independently selected from H and F. In some such embodiments, s is 1 and R^{c} and R^{c′} are either both H or are both F. In some embodiments, both R^{c} and R^{c′} are H.

In some embodiments of the compounds of formula II, s is 1.

In some embodiments of the compounds of formula II, r is 0.

In some embodiments of the compounds of formula II, R⁴ is an unsubstituted phenyl group or is a phenyl group that is substituted with at least one cyano, halo, —CF₃, C₁-C₆ alkyl, —OH, or C₁-C₆ alkoxy group. In some such embodiments, R⁴ is a phenyl group substituted with a methyl group. In some such embodiments, R⁴ is a phenyl group substituted in the para position with a methyl group

In some embodiments of the compounds of formula II, R³ is a C₁-C₆ alkyl group. In some such embodiments, R³ is a methyl, ethyl, or propyl group. In some of these embodiments, R³ is a methyl group.

In some embodiments of the compounds of formula II, C is a 5 or 6 membered carbocyclic or heterocyclic ring. In some such embodiments, C is a 5 or 6 membered carbocyclic ring. In other embodiments, C is heterocyclic ring that includes one heteroatom selected from N, O or S. In some embodiments, the C ring is not an aromatic ring and is at least partially saturated.

In some embodiments of the compounds of formula II, the C ring is substituted with a C₁-C₆ alkyl group such as a methyl, ethyl, propyl, or butyl group. In some such embodiments, the C ring is substituted with a methyl group. In other embodiments, the C ring does not include any further substituents.

In some embodiments of the compounds of formula II, the fragment D has a formula selected from: 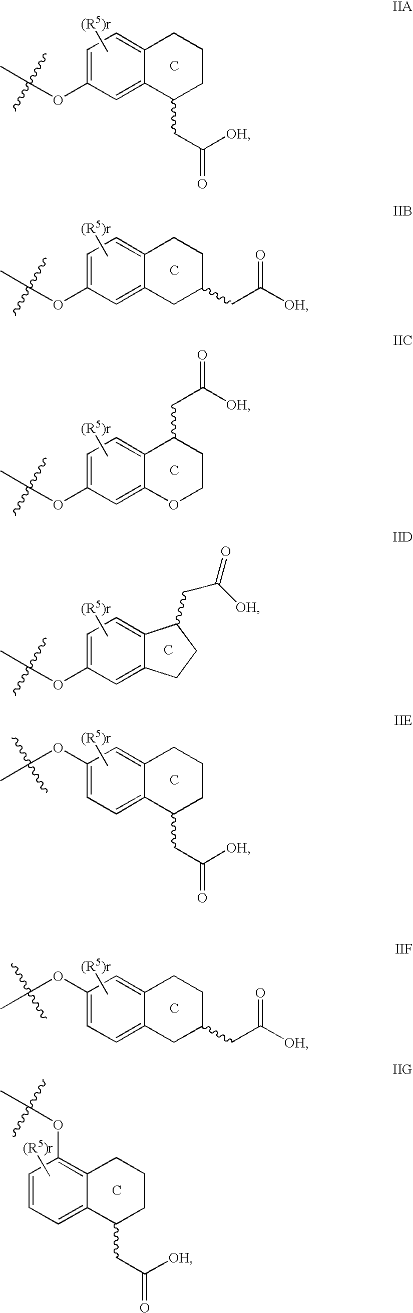 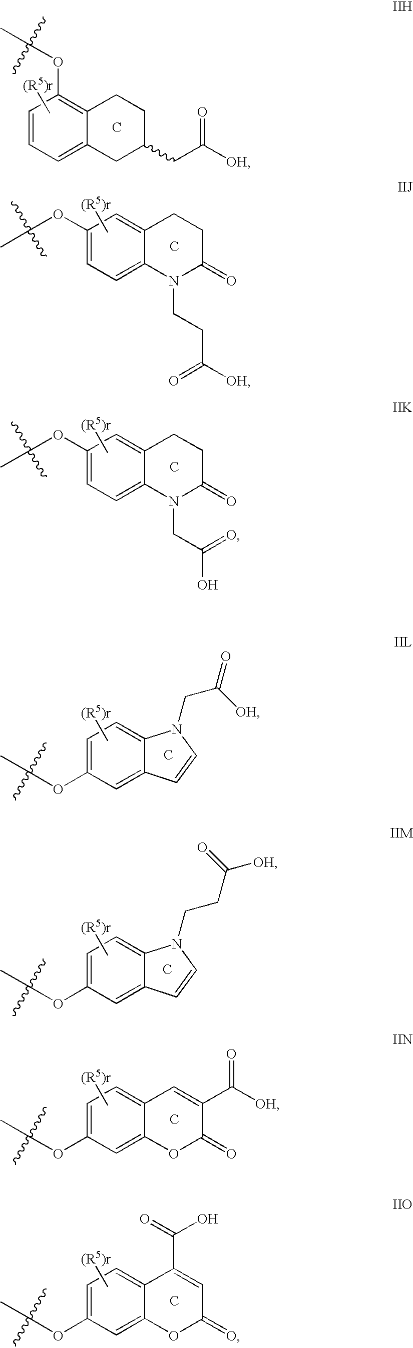 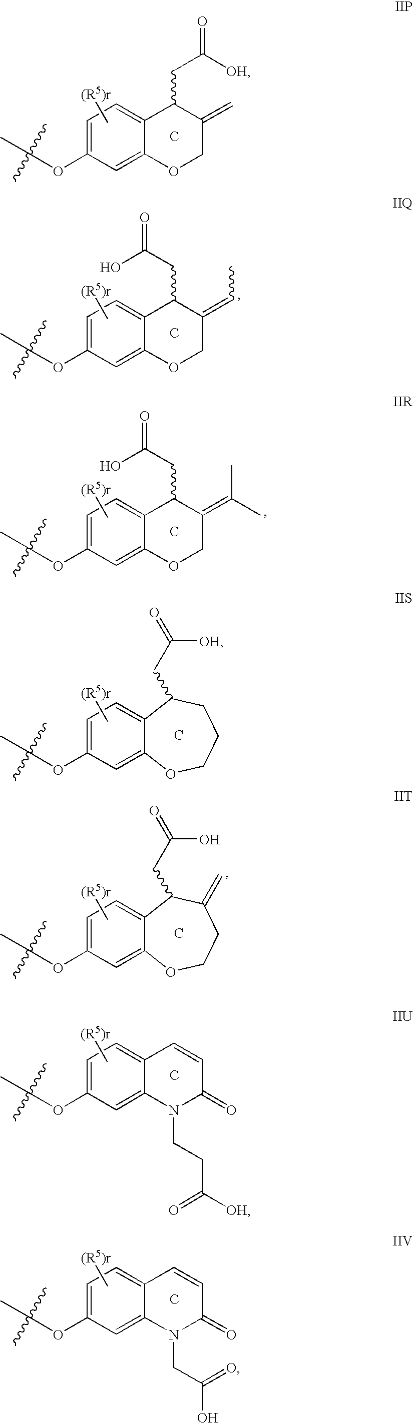 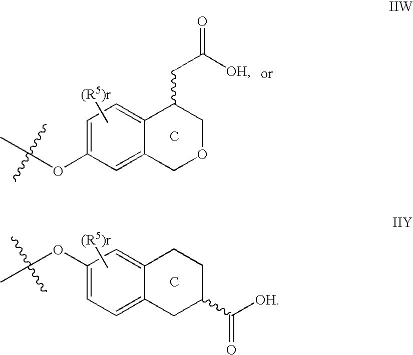
In such embodiments, the C ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups. In the structures shown above, a wavy bond indicates a point of attachment when drawn across a bond, indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers. In some embodiments, the compound has the formula of any one or more of the structures shown above.

In another aspect, the invention provides compounds of formula III
F-L₁-E-L₂-L₃-G III
and pharmaceutically acceptable salts, esters, solvates, tautomers, stereoisomers, and/or prodrugs thereof,
wherein,
E is selected from an aryl group or a heterocyclyl group;
F is selected from —H, an aryl group, or a heterocyclyl group;
L₁ is selected from a bond, —O—, —NH—, —S—, —CH₂—, —C(═O)—, —SO—, or —SO₂—;
L₂ is selected from —(CH₂)ₘ—, or O—(CH₂)ₘ— where m is selected from 1 or 2;
L₃ is —O—, —NH—, —S—, or L₂ and L₃, when taken together, represent a group of formula —CH═CH—, or —C(═CH₂)—; and
G is selected from 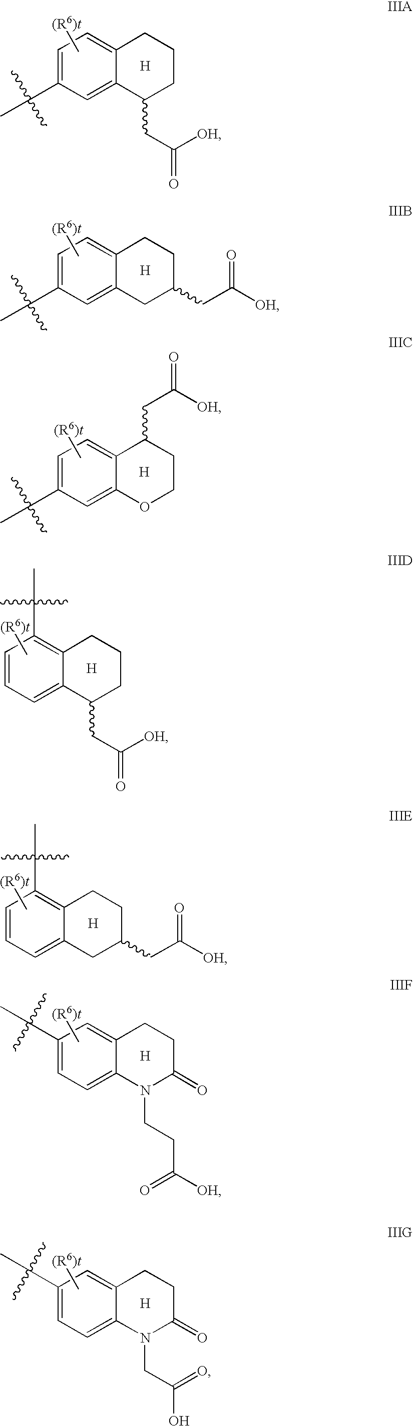 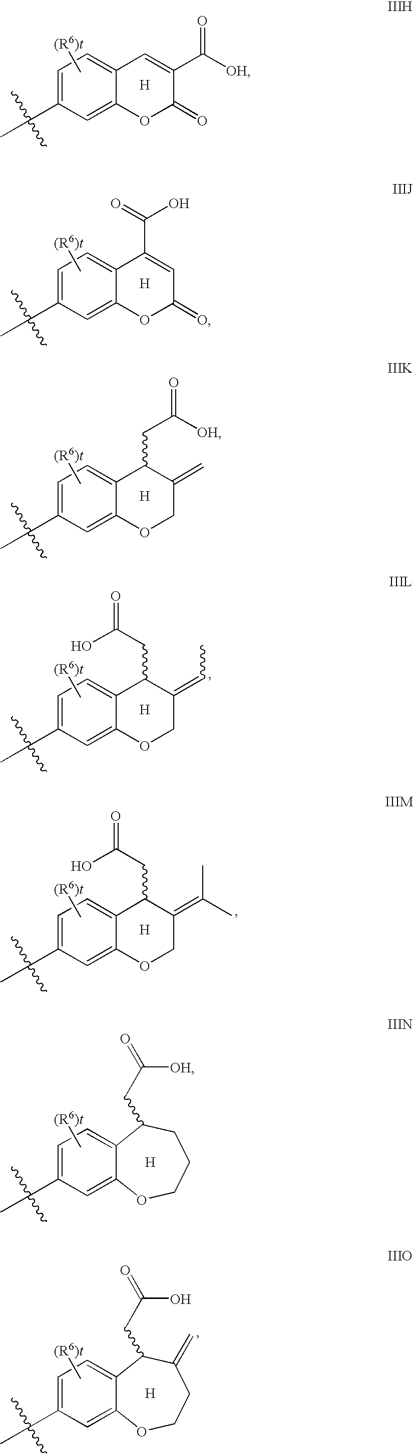 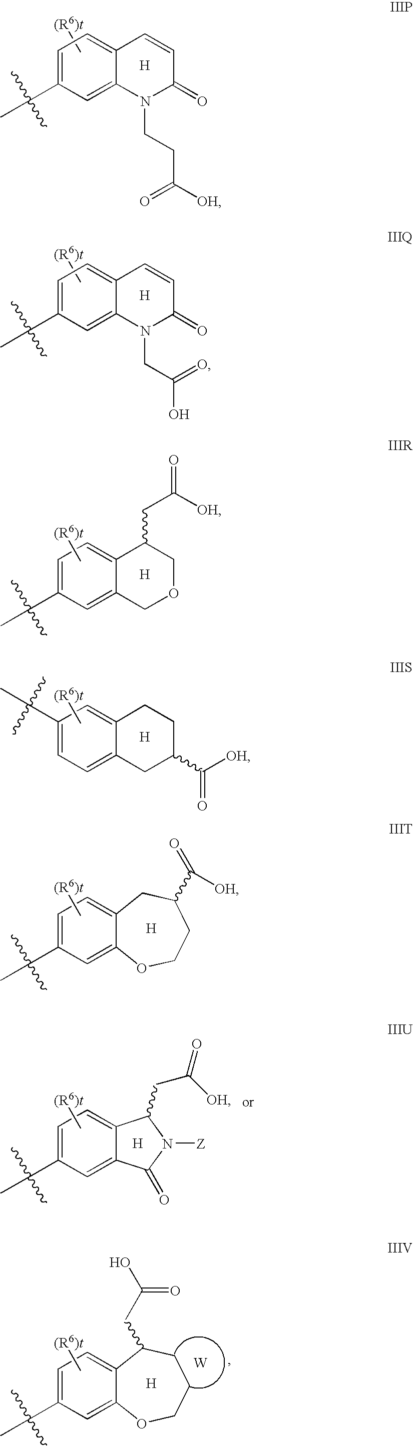
   wherein,
R⁶ is selected from halo, C₁-C₆ alkyl, —OH, or C₁-C₆ alkoxy;
t is selected from 0, 1, or 2;
each R⁶ is independently selected if t is 2,
Z is selected from H and C₁-C₆ alkyl; and
W is a 5 to 7 membered heterocyclic ring.
   The H ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups. A wavy bond indicates a point of attachment when drawn across a bond, indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers. If G is IIIT, L³ is —O—, L² is —(CH₂)—, L¹ is a bond, E is an unsubstituted benzene ring, and F and L² are oriented in a meta substitution pattern on E, then F is not substituted with two methyl groups. If G is IIIT, L³ is —O—, L² is —(CH₂)—, L¹ is —O—, E is an unsubstituted benzene ring, and L¹ and L² are oriented in a meta substitution pattern on E, then F is not an unsubstituted benzene ring. Each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl.
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl.

In some embodiments of the compounds of formula III, L₁ is a bond or —O—. In some such embodiments, L₁ is a bond. In other such embodiments, L₁ is —O—.

In some embodiments of the compounds of formula III, L₃ is —O—, or L₂ and L₃, when taken together, represent a group of formula —CH═CH—, or —C(═CH₂)—.

In some embodiments of the compounds of formula III, L₃ is —O—. In some embodiments, L₃ is —O—; L₂ is —(CH₂)ₘ— and m is 1; E is an optionally substituted phenyl or thiazole. In some such embodiments L₁ is a bond, and F is an optionally substituted phenyl.

In some embodiments of the compounds of formula III, L₂ is —CH₂)ₘ— and m is 1.

In some embodiments of the compounds of formula III, E is an optionally substituted thiazole group. In some such embodiments, the compound has the formula IV where R⁷ is selected from —H, halo, or C₁-C₆ alkyl and the other variables have any of the definitions of the other embodiments 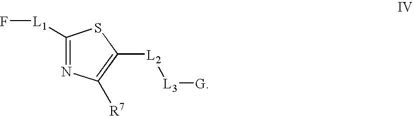
In some such embodiments, R⁷ is a C₁-C₆ alkyl groups such as a methyl group.

In some embodiments of the compounds of formula III, E is an optionally substituted phenyl group. In some such embodiments, the compound has the formula VA or VB where R⁸ is selected from halo, cyano, C₁-C₆ alkyl, —OH, or C₁-C₆ alkoxy; u is selected from 0, 1, or 2; each R⁸ is independently selected if u is 2, and the other variables have any of the values of the other embodiments

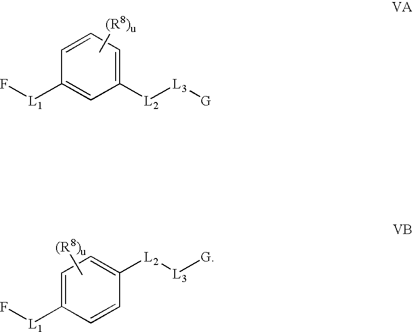

In some embodiments of the compounds of formula III, F is an unsubstituted phenyl group or is a phenyl group that is substituted with at least one cyano, —CF₃, C₁-C₆ alkyl, —OH, or C₁-C₆ alkoxy group. In some such embodiments, F is a phenyl group substituted with at least one methyl group, methoxy group, ethoxy group, propoxy group, butoxy group, or pentoxy group.

In some embodiments of the compounds of formula III, if G is selected from IIIT, then F is not an aryl group substituted with two methyl groups.

In some embodiments of the compounds of formula III, G is selected from one of IIIA-IIIS. In other embodiments, G is selected from one of IIIT, IIIU, or IIIV. In some embodiments where G is IIIU, X is H whereas in other such embodiments, Z is methyl.

In some embodiments of the compounds of formula III where G is IIIV, W is a heterocyclic ring having 5 or 6 ring members. In some such embodiments, W is a heteroaryl ring. In some such embodiments, W is an isoxazole. In some such embodiments, IIIV, has the formula IIIV′.

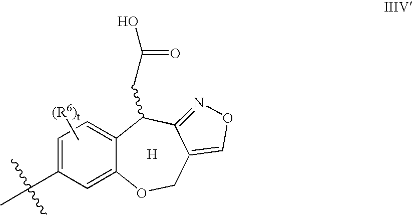

In another aspect, the invention provides compounds of formula VI 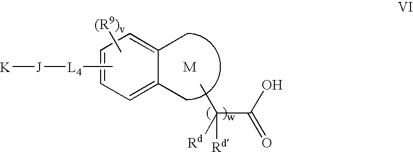
and pharmaceutically acceptable salts, esters, solvates, tautomers, stereoisomers, and/or prodrugs thereof,
wherein,
J is selected from an aryl group or a heterocyclyl group;
K is selected from —H, —CF₃, halo, cyano, C₁-C₆ alkyl, —OH, C₁-C₆ alkoxy, —O-aryl, an aryl group, or a heterocyclyl group;
M is a 5 to 7 membered carbocyclic or heterocyclic ring;
L₄ is selected from —CH₂CH₂—, —CH═CH—, or —C(═CH₂)—;
R⁹ is selected from halo, C₁-C₆ alkyl, —OH, or C₁-C₆ alkoxy;
v is selected from 0, 1, or 2;
w is selected from 0, 1, or 2;
each R⁹ is independently selected if v is 2; and
R^{d} and R^{d′} are independently selected from —H and halo, and further wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl,
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
and further wherein the M ring may be further substituted with an oxo group or a group of formula ═CR^{a}R^{a′} where R^{a} and R^{a′} are independently selected from H or C₁-C₄ alkyl groups.

In some embodiments of the compounds of formula VI, R^{d} and R^{d′} are independently selected from H and F. In some such embodiments, w is I and R^{d} and R^{d′} are either both H or are both F. In some embodiments, both R^{d} and R^{d′} are H.

In some embodiments of the compounds of formula VI, w is 1.

In some embodiments of the compounds of formula VI, v is 0.

In some embodiments of the compounds of formula VI, J is an optionally substituted aryl group.

In some embodiments of the compounds of formula VI, J is an optionally substituted thiazole group.

In some embodiments of the compounds of formula VI, M is a 6 membered carbocyclic or heterocyclic ring. In some such embodiments, M is a 6 membered carbocyclic ring. In other embodiments, M is heterocyclic ring that includes one heteroatom selected from N, O or S. In some embodiments, the M ring is not an aromatic ring and is at least partially saturated.

In some embodiments of the compounds of formula VI, the M ring is substituted with a C₁-C₆ alkyl group such as a methyl, ethyl, propyl, or butyl group. In some such embodiments, the M ring is substituted with a methyl group. In other embodiments, the M ring does not include any further substituents.

In some embodiments of the compounds of formula I, II, III, and/or VI, the B ring, the C ring, the H ring, or the M ring is substituted with a ═CR^{a}R^{a′} group where R^{a} and R^{a′} are independently selected from H and C₁-C₄ alkyl groups.

In another aspect, the invention provides compounds of formula VII 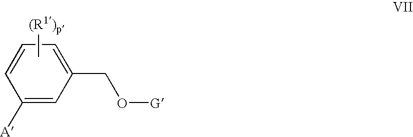
or a pharmaceutically acceptable salt, ester, solvate, tautomer, stereoisomer, or prodrug thereof,
wherein,
A′ is selected from an aryl group or a heterocyclyl group;
R^{1′} is selected from halo, cyano, C₁-C₆ alkyl, —OH, or C₁-C₆ alkoxy;
p′ is selected from 0, 1, or 2;
each R^{1′} is independently selected if p is 2; and
G′ is selected from 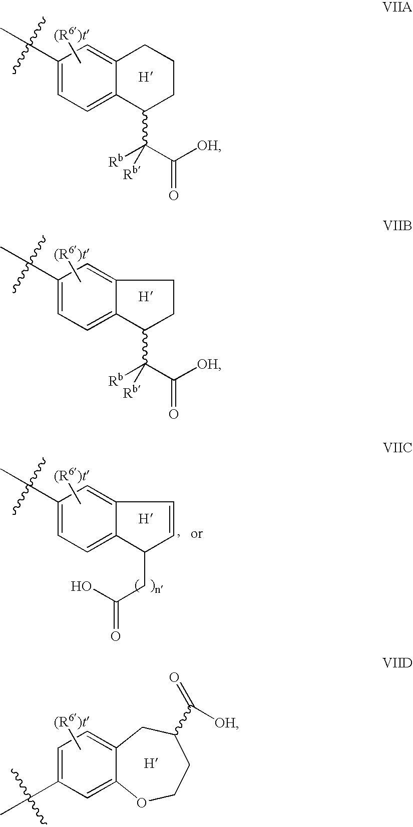
   wherein,
R^{6′} is selected from halo, C₁-C₆ alkyl, —OH, or C₁-C₆ alkoxy;
t′ is selected from 0, 1, or 2;
each R^{6′} is independently selected if t′ is 2;
R^{b} and R^{b′} are independently selected from —H and halo; and
n′ is selected from 1 or 2
   and further wherein the H′ ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups, and a wavy bond indicates a point of attachment when drawn across a bond, or indicates the R and S enantiomers individually or as a mixture of the R and S enantiomers;
   and further wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
   C₁-C₆ alkoxy,
   C₁-C₆ alkyl optionally substituted by halo,
   aryl,
   halo,
   hydroxyl,
   heteroaryl,
   C₁-C₆ hydroxyalkyl, or
   —NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
   and further wherein, A′ does not have the following formula

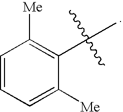

In some embodiments of the compounds of formula VII, A′ is a phenyl group that is substituted with at least one cyano, —CF₃, C₁-C₆ alkyl, —OH, or C₁-C₆ alkoxy group.

In some embodiments of the compounds of formula VII, A′ is a phenyl group that is substituted with at least one —CF₃, —F, —Cl, —Br, —I, methoxy group, ethoxy group, propoxy group, butoxy group, or pentoxy group.

In some embodiments of the compounds of formula VII, p′ is 0.

In some embodiments of the compounds of formula VII, t′ is 0.

In some embodiments of the compounds of formula VII, G′ is VIIA. In other embodiments, G′ is VIIB. In still other embodiments, G′ is VIIC. In still other embodiments, G′ is VIID.

In some embodiments of the compounds of formula VII, H′ is not further substituted.

In some embodiments of the compounds of formula VII, H′ is substituted with a C₁-C₄ alkyl group.

In some embodiments of the compounds of formula VII, H′ is substituted with a group of formula ═CR^{a}R^{a′} where R^{a} and R^{a′} are independently selected from H or C₁-C₄ alkyl groups.

In other aspects, the invention provides pharmaceutical compositions that include a pharmaceutically acceptable carrier, diluent or excipient and any of the compounds, or pharmaceutically acceptable salts, esters, solvates, tautomers, stereoisomers, and/or prodrugs thereof of any of the embodiments described herein. In other aspects the invention thus also provides the use of any of the compounds, or pharmaceutically acceptable salts, esters, solvates, tautomers, stereoisomers, and/or prodrugs thereof of the invention in the preparation of a medicament. Such medicaments may be used in accordance with the methods described herein.

In some embodiments, the compound of the invention comprises a stereomerically pure stereoisomer. In other embodiments, the compounds comprise a mixture of stereoisomers. In some embodiments, the compound comprises a stereomerically pure S-enantiomer. In other embodiments, the compound comprises a stereomerically pure R-enantiomer. In yet other embodiments, the compound comprises a mixture of S- and R-enantiomers.

In another aspect, the invention provides pharmaceutical compositions suitable for pharmaceutical use comprising one or more compound(s) of the invention and a pharmaceutically acceptable carrier, excipient or diluent.

The term “composition” as used herein is intended to encompass a product comprising the specified ingredients (and in the specified amounts, if indicated), as well as any product which results, directly or indirectly, from combination of the specified ingredients in the specified amounts. The term “pharmaceutically acceptable” means that the carrier or excipient is compatible with the other ingredients of the formulation and is not deleterious to the recipient thereof.

Composition formulation may improve one or more pharmacokinetic properties (e.g., oral bioavailability, membrane permeability) of a compound of the invention (herein referred to as the active ingredient).

Pharmaceutical compositions for use in administering the compounds of the invention may conveniently be presented in unit dosage form and may be prepared by any of the methods well known in the art. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

Pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,160,452 and 4,265,874 to form osmotic therapeutic tablets for control release.

Formulations for oral administration and use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Pharmaceutical compositions may also be prepared in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Examples of such materials include, but are no limited to, cocoa butter and polyethylene glycols.

For topical use, creams, ointments, jellies, solutions, suspensions, and the like, containing the compounds may be prepared and employed. As used herein, topical application is also meant to include the use of mouthwashes and gargles.

The pharmaceutical compositions and methods of the invention may further comprise other therapeutically active compounds, as noted herein, useful in the treatment of type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer and edema.

In another aspect, the invention provides methods of treating or preventing a disease or condition selected from the group consisting of type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer and edema,. Such methods include administering to a subject in need thereof a therapeutically effective amount of a compound or composition of the invention. In some embodiments, the disease or condition is type II diabetes.

In another aspect, the present invention provides a method for treating a disease or condition responsive to the modulation of GPR40. Such methods include administering to a subject in need thereof a therapeutically effective amount of a compound or composition of the invention.

In some embodiments, the disease or condition is selected from the group consisting of type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer, and edema.

In certain embodiments, the disease or condition is type II diabetes.

In some embodiments, the disease or condition is obesity.

In some embodiments, the disease or condition is hypertension.

As indicated, the compounds and compositions of the invention may be administered in various ways. For example, in some embodiments, a compound or composition of the invention is administered orally, parenterally, or topically. In some such embodiments, the compound or composition is administered orally. In other embodiments, the compound or composition is administered parenterally. In still other embodiments, the compound or composition is administered topically.

In some embodiments, the compound or composition is administered in combination with a second therapeutic agent. In some such embodiments, the second therapeutic agent is an insulin sensitizing agent, such as metformin or a thiazolidinedione, for example. The second therapeutic agent may be administered before, during or after the compound or composition of the invention is administered to a subject.

In another aspect, the invention provides methods for treating or preventing a disease or disorder responsive to modulation of GPR40. Such methods include administering a therapeutically effective amount of one or more of the subject compounds or compositions to a subject having such a disease or disorder.

In yet another aspect, the invention provides methods for treating or preventing a GPR40-mediated condition, disease or disorder. Such methods include administering a therapeutically effective amount of one or more of the subject compounds or compositions to a subject having such a condition, disease or disorder.

In yet another aspect, the invention provides methods for modulating GPR40. Such methods include contacting a cell with one or more of the compounds or compositions of the invention. For example, in some embodiments, a cell that constitutively expresses GPR40 is contacted with one or more of the subject compounds or compositions.

In certain embodiments, a cell to be contacted can be made to express or overexpress GPR40, for example, by expressing GPR40 from heterologous nucleic acid introduced into the cell or, as another example, by upregulating the expression of GPR40 from nucleic acid endogenous to the cell.

Depending on the disease to be treated and the subject's condition, the compounds of the invention may be administered by oral, parenteral (e.g., intramuscular, intraperitoneal, intravenous, ICV, intracisternal injection or infusion, subcutaneous injection or implant), inhalation, nasal, vaginal, rectal, sublingual, or topical (e.g., transdermal, local) routes of administration and may be formulated, alone or together, in suitable dosage unit formulations containing conventional non-toxic pharmaceutically acceptable carriers, adjuvants and vehicles appropriate for each route of administration. The invention also contemplates administration of the compounds of the invention in a depot formulation, in which the active ingredient is released over a defined time period.

In the treatment or prevention type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer and edema or other conditions or disorders associated with GPR40, an appropriate dosage level will generally be about 0.001 to 100 mg per kg patient body weight per day which can be administered in single or multiple doses. In some embodiments, the dosage level will be about 0.01 to about 25 mg/kg per day, whereas in other embodiments the dosage level will be about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day. In some such embodiments that are administered once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of the invention can be combined or used in combination with other agents useful in the treatment, prevention, suppression or amelioration of the diseases or conditions for which compounds of the invention are useful, including type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer and edema. Such other agents, or drugs, may be administered, by a route and in an amount commonly used therefore, simultaneously or sequentially with a compound of the invention. When a compound of the invention is used contemporaneously with one or more other drugs, a pharmaceutical composition containing such other drugs in addition to the compound of the invention may be prepared. Alternatively, the other drug may be administered to a subject from a composition other than one that includes a compound of the invention. Accordingly, the pharmaceutical compositions of the invention include those that also contain one or more other active ingredients or therapeutic agents, in addition to a compound of the invention.

Examples of other therapeutic agents that may be combined with a compound of the invention, either by separate administration, or in the same pharmaceutical composition, include, but are not limited to: (a) cholesterol lowering agents such as HMG-CoA reductase inhibitors (e.g., lovastatin, simvastatin, pravastatin, fluvastatin, atorvastatin and other statins), bile acid sequestrants (e.g., cholestyramine and colestipol), vitamin B₃ (also known as nicotinic acid, or niacin), vitamin B₆ (pyridoxine), vitamin B₁₂ (cyanocobalamin), fibric acid derivatives (e.g., gemfibrozil, clofibrate, fenofibrate and benzafibrate), probucol, nitroglycerin, and inhibitors of cholesterol absorption (e.g., beta-sitosterol and acylCoA-cholesterol acyltransferase (ACAT) inhibitors such as melinamide), HMG-CoA synthase inhibitors, squalene epoxidase inhibitors and squalene synthetase inhibitors; (b) antithrombotic agents, such as thrombolytic agents (e.g., streptokinase, alteplase, anistreplase and reteplase), heparin, hirudin and warfarin derivatives, β-blockers (e.g., atenolol), β-adrenergic agonists (e.g., isoproterenol), ACE inhibitors and vasodilators (e.g., sodium nitroprusside, nicardipine hydrochloride, nitroglycerin and enaloprilat); and (c) anti-diabetic agents such as insulin and insulin mimetics, sulfonylureas (e.g., glyburide, meglinatide), biguanides, e.g., metformin (GLUCOPHAGE®), α-glucosidase inhibitors (acarbose), insulin sensitizers, e.g., thiazolidinone compounds, rosiglitazone (AVANDIA®), troglitazone (REZULIN®), ciglitazone, pioglitazone (ACTOS®) and englitazone, DPP-IV inhibitors, e.g., vildagliptin (Galvus®), sitagliptin (Januvia™), and GLP-I analogs, e.g., exenatide (Byetta®). In some embodiments, a compound of the invention may be administered along with a DPP-IV inhibitor or a GLP-I analog.

The weight ratio of the compound of the invention to the second active ingredient may be varied and will depend upon the effective dose of each ingredient. Generally, an effective dose of each will be used. Combinations of a compound of the invention and other active ingredients will generally also be within the aforementioned range, but in each case, an effective dose of each active ingredient should be used.

In another aspect, the invention provides a method for modulating circulating insulin concentration in a subject. Such methods include administering a compound or composition of the invention to the subject. In some embodiments, the insulin concentration is increased whereas in other embodiments, the insulin concentration is decreased.

The following examples are offered by way of illustration and are not intended to limit the scope of the invention. Those of skill in the art will readily recognize a variety of noncritical parameters that could be modified to yield essentially similar results.

### EXAMPLES

Unless otherwise stated, all compounds were obtained from commercial sources or were prepared using the methods and experimental procedures described herein. The following Abbreviations are used to refer to various reagents and solvents:
AIBN Azobisisobutyronitrile
BINAP 2,2′-Bis(diphenylphosphino)-1,1′-binaphthyl)
DCM Dichloromethane
DBU 1,8-Diazabicyclo[5.4.0]undec-7-ene
DIAD Diisopropyl azodicarboxylate
DME Dimethoxyethane
DMF N,N-Dimethyl Formamide
EtOAc Ethyl Acetate
EtOH Ethanol
IPA Isopropanol
LDA Lithium Diisopropylamide
LiHMDS Lithium Hexamethyidisilazide
MeI Methyl Iodide
MeOH Methanol
PPh₃ Triphenylphosphine
THF Tetrahydrofuran

### Section I

The synthesis of benzyl halides A-I is described in the following section.

### Example A

This example illustrates the preparation of 3-(4-trifluoromethylphenyl)-benzyl chloride (1).

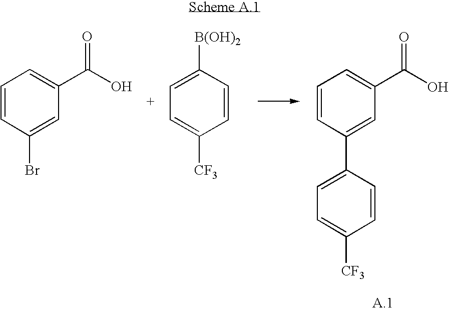

3-(4-Trifluoromethylphenyl)-benzoic acid (A.1). The Suzuki coupling was carried out according to the method of Dyer et al. (Tetrahedron Letters, 42:1765-1767 (2001)). Commercially available 4-(trifluoromethyl)phenylboronic acid (15 g, 78.7 mmol) and 3-bromobenzoic acid (15.1 g, 75 mmol) were suspended in 2-propanol:water (1:4, 72 mL). 10% Pd/C (1.5 g) was added followed by aqueous Na₂CO₃ (39 mL, 20% by wt.). The resulting mixture was heated at 70° C. for 4 hours. The precipitate was filtered and rinsed with a 20% aqueous Na₂CO₃ solution. The filtrate was diluted with water and acidified to a pH of about 2. The white solid was filtered and dried in vacuo. The crude material (A.1) (19.69 g) was used in the next step without further purification.

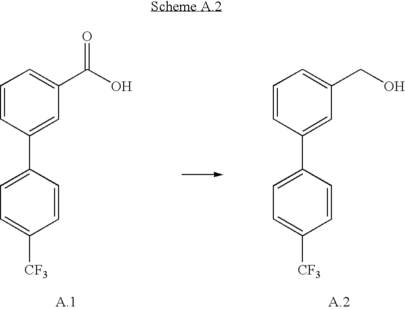

3-(4-Trifluoromethylphenyl)-benzyl alcohol (A.2). The carboxylic acid A.1 (13.3 g, 50 mmol) in anhydrous THF (100 mL) was added dropwise to LiAlH₄ (2.9 g, 75 mmol) in anhydrous THF (150 mL) at 0° C. over 30 minutes. The resulting mixture was slowly warmed to room temperature and stirred for 4 hours. The reaction was slowly quenched with water (2.9 mL) at 0° C., 15% NaOH aqueous solution (2.9 mL), and another portion of water (8.7 mL). The mixture was dried over Na₂SO₄ and concentrated to give a white solid (11.9 g). The crude product (A.2) was used in the next step without further purification.

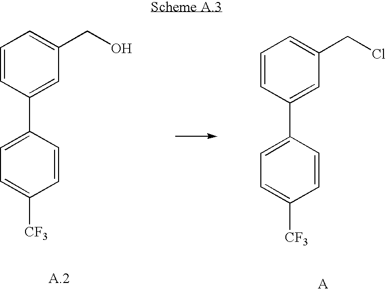

3-(4-Trifluoromethylphenyl)-benzyl chloride (A). The alcohol A.2 (15 g, 59.5 mmol) was dissolved in anhydrous DCM (100 mL). Thionyl chloride (10 mL) was slowly added dropwise to the above solution. The resulting mixture was stirred at room temperature for 24 hours. The organic solvent was removed under vacuo. The residue was then purified by flash chromatography (SiO₂ gel 60, eluted with 20% DCM in hexanes). Fractions containing the desired product A were combined and concentrated to provide the desired product as a white solid (14.0 g). ¹H NMR (400 MHz) (CDCl₃) δ 7.73 (4H, s); 7.65 (1H, s); 7.58 (1H, s); 7.52-7.28 (2H, m); 4.69 (2H, s).

### Example B

This example illustrates the preparation of 4-(3-ethoxyphenyl)benzyl chloride (B).

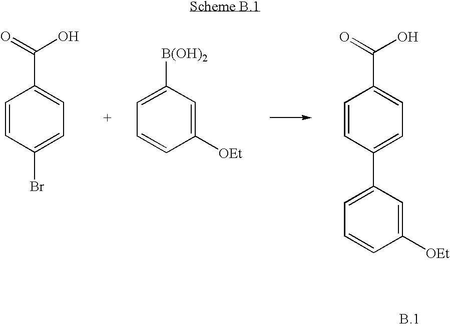

4-(3-Ethoxyphenyl)benzoic acid (B.1). Commercially available 3-ethoxyphenyl boronic acid (15.0 g, 90.4 mmol) and 4-bromobenzoic acid (15.0 g, 75.3 mmol) were suspended in 2-propanol:water (1:1,200 mL). 10% Pd/C (1.5 g) was added followed by Na₂CO₃ (9.60 g, 90.4 mmol). The resulting mixture was heated at 80° C. for 24 hours. The mixture was filtered through Celite® and rinsed with water. The filtrate was acidified to a pH of about 2. The white solid was filtered and dried in vacuo. The crude material (B.1) (18.0 g) was used in the next step without further purification.

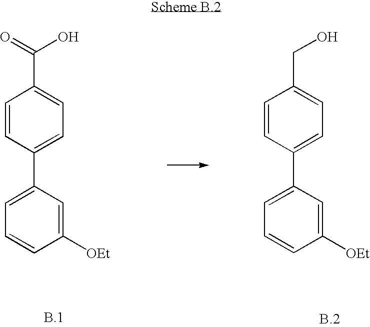

4-(3-Ethoxyphenyl)-benzyl alcohol (B.2). To the carboxylic acid B.1 (18.0 g, 74.3 mmol) in anhydrous THF (200 mL) was added dropwise a solution of LiAlH₄ in THF (1.0 M, 157 mL, 157 mmol) at 0° C. over 30 minutes. The resulting mixture was slowly warmed to room temperature and stirred for 16 hours. The reaction was slowly quenched with water (10 mL) at 0° C., 1N NaOH aqueous solution (50 mL), and another portion of water (10 mL). The mixture was filtered and the filtrate was extracted with EtOAc (3×100 mL), dried over MgSO₄ and concentrated to give the product as a colorless oil (15.5 g). The crude product (B.2) was used in the next step without further purification.

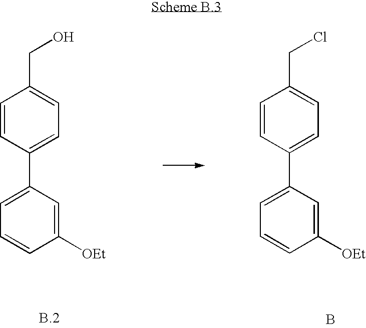

4-(3-Ethoxyphenyl)-benzyl chloride (B). The alcohol B.2 (15.5 g, 67.9 mmol) was dissolved in anhydrous DCM (200 mL). Thionyl chloride (9.9 mL, 136 mmol) was slowly added dropwise to the above solution. The resulting mixture was stirred at room temperature for 16 hours. The organic solvent was removed in vacuo. The residue was then purified by flash chromatography (SiO₂ gel 60, eluted with 10% EtOAc in hexanes). Fractions containing the desired product B were combined and concentrated to a colorless solid (15.0 g, 90%). ¹H NMR (500 MHz) (CDCl₃) δ 7.60 (2H, d); 7.48 (2H, d); 7.37 (1H, m); 7.18 (1H, d), 7.14 (1H, s), 6.92 (1H, d), 4.67 (2H, s), 4.12 (2H, q), 1.47 (3H, t).

Compounds C-1 were prepared using methods similar to those used to prepare compounds A and B. Further relevant synthetic routes for related compounds are described in WO 2005/086661 and US 2006/0004012 which are both hereby incorporated by reference in their entireties and for all purposes as if fully set forth herein.

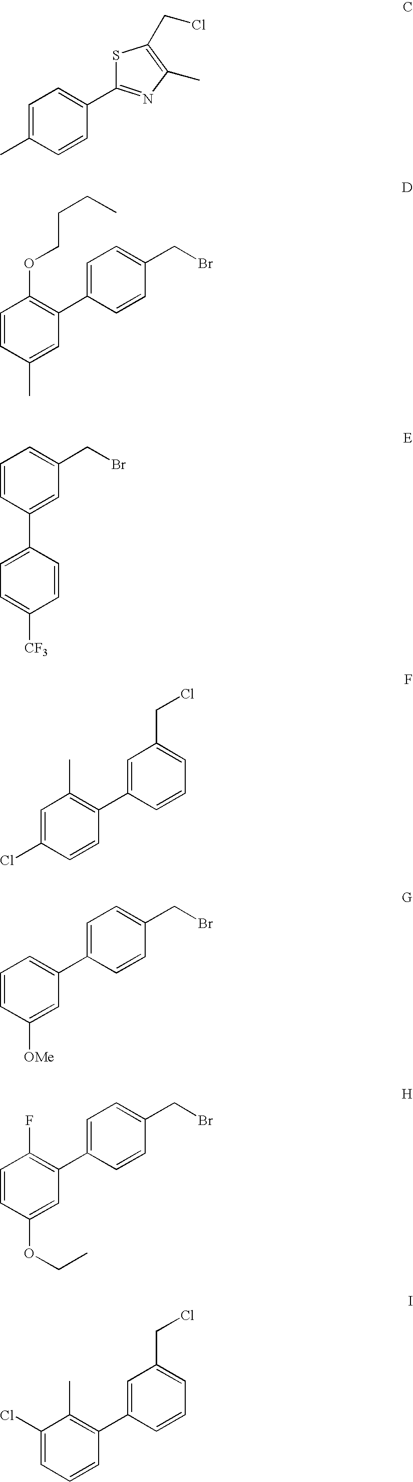

### Section II

This section provides the experimental procedures used to synthesize the examples shown in the Table that follows.

### Example 1

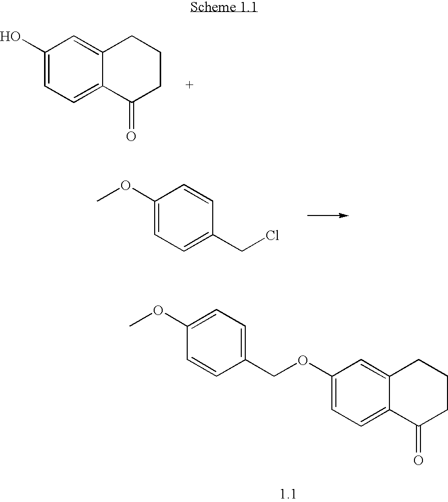

6-(4-Methoxy-benzyloxy)-3,4-dihydro-2H-naphthalen-1-one (1.1). 6-Hydroxy-1-tetralone (3.244 g, 20 mmol) and 4-methoxybenzyl chloride (3.132 g, 20 mmol) were dissolved in DMF (20 mL). Cs₂CO₃ (7.168 g, 22 mmol) was added to the mixture. The resulting mixture was stirred overnight at ambient temperature. The reaction mixture was then diluted with water (200 mL) and extracted with EtOAc (50 mL×3). The combined organic layers were washed with saturated brine, dried over MgSO₄, filtered, and concentrated under reduced pressure. The residue was used without further purification in the next step. MS ESI (pos.) m/e: 283 (M+1)⁺.

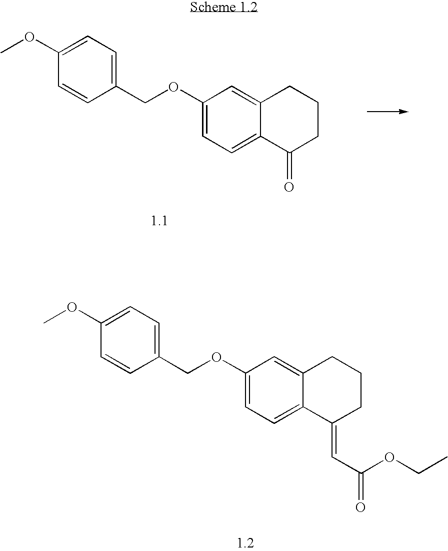

[6-(4-Methoxy-benzyloxy)-3,4-dihydro-2H-naphthalen-(1E)-ylidene]-acetic acid (1.2). A solution of ethyl (trimethylsilyl)acetate (12.065 g, 75 mmol) in anhydrous THF (60 mL) at −78° C. was treated with LiHMDS (1M in THF, 66 mL) dropwise. The resulting mixture was stirred at −78° C. for 30 minutes. A solution of 1.1 (14 g, 50 mmol) in THF (10 mL) was added to the mixture over 20 minutes. The resulting mixture was stirred at −78° C. for 2 hours before being warmed to 0° C. The reaction mixture was quenched with saturated NH₄Cl (200 mL) and extracted with EtOAc (70 mL×4). The combined organic layers were washed with saturated brine, dried over MgSO₄, filtered, and concentrated under reduced pressure. The residue was purified by flash chromatography (silica gel, 30% EtOAc in hexane). [6-(4-Methoxy-benzyloxy)-3,4-dihydro-2H-naphthalen-(1E)-ylidene]-acetic acid (1.2) was obtained as a light yellow oil (12.2 g, 69% yield). MS ESI (pos.) m/e: 353 (M+1)⁺.

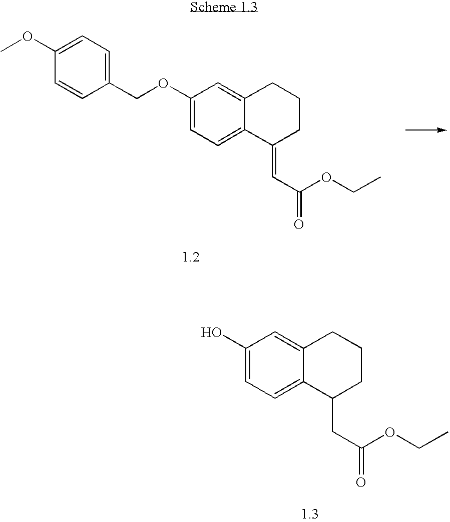

(R/S)-(6-Hydroxy-1,2,3,4-tetrahydro-naphthalen-1-yl)-acetic acid ethyl ester (1.3). Compound 1.2 (6.2 g, 17.6 mmol) was dissolved in MeOH (30 mL). Pd/C (10%, 620 mg) was carefully added to the solution. A H₂ balloon was attached to the reaction flask. The resulting mixture was stirred overnight at ambient temperature. The Pd/C was removed by filtration, and the filtrate was concentrated under reduced pressure. (R/S)-(6-Hydroxy-1,2,3,4-tetrahydro-naphthalen-1-yl)-acetic acid ethyl ester (1.3) was obtained as a clear oil (3.753 g, 91%). MS ESI (pos.) m/e: 235 (M+1)⁺ and 257 (M+Na)⁺. ¹H NMR (400 MHz) (CDCl₃) δ 7.02 (d, 1H); 6.64 (d, 1H); 6.56 (s, 1H); 5.04 (d, 1H); 4.20 (q, 2H); 3.31 (m, 1H); 2.73 (m, 2H); 2.54 (m, 2H); 1.71 (m, 4H); 1.29 (t, 3H).

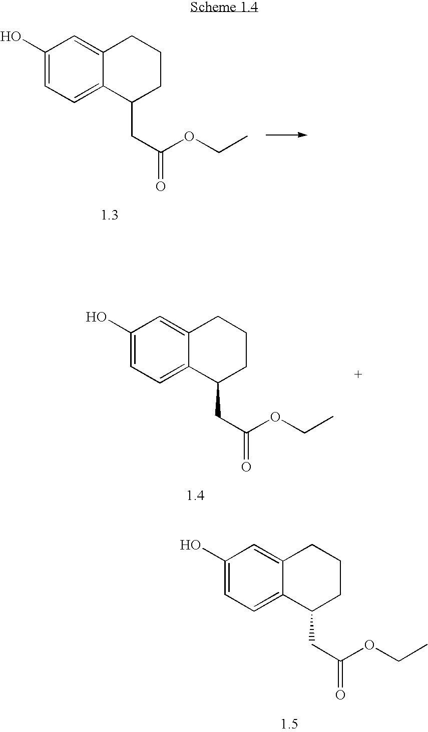

((S)-6-Hydroxy-1,2,3,4-tetrahydro-naphthalen-1-yl)-acetic acid ethyl ester (1.4) and ((R)-6-hydroxy-1,2,3,4-tetrahydro-naphthalen-1-yl)-acetic acid ethyl ester (1.5) were obtained by separating 1.3 using a preparatory AD-H column (7% IPA in hexanes).

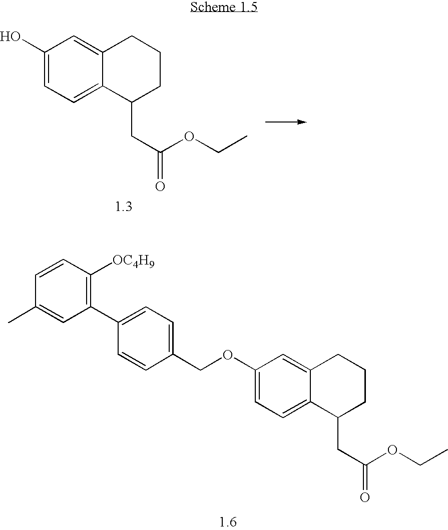

(R/S)-[6-(2′-Butoxy-5′-methyl-biphenyl-4-ylmethoxy)-1,2,3,4-tetrahydro-naphthalen-1-yl]-acetic acid ethyl ester (1.6). A mixture of 1.3 (46.8 mg, 0.2 mmol), D (67 mg, 2 mmol) and Cs₂CO₃ (98 mg, 0.3 mmol) in DMF (1 mL) was stirred at ambient temperature for 2 hours. The reaction mixture was quenched with water (10 mL) and extracted with EtOAc (5 mL×3). The combined organic layers were washed with saturated brine, dried over MgSO₄, filtered, and concentrated under reduced pressure. The residue was used without further purification. (R/S)-[6-(2′-Butoxy-5′-methyl-biphenyl-4-ylmethoxy)-1,2,3,4-tetrahydro-naphthalen-1-yl]-acetic acid ethyl ester (1.6) was obtained as a thick oil (83 mg, 85%). LC-MS ESI (pos.) m/e: 487.1 (M+H)⁺.

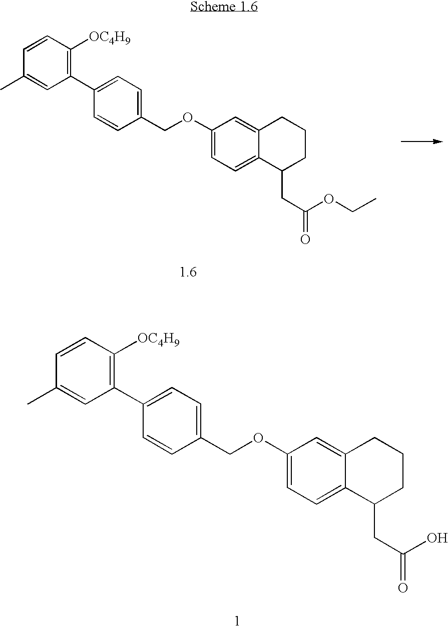

(R/S)-[6-(2′-Butoxy-5′-methyl-biphenyl-4-ylmethoxy)-1,2,3,4-tetrahydro-naphthalen-1-yl]-acetic acid (1): A mixture of 1.6 (83 mg, 0.17 mmol) in NaOH solution (3%, THF/MeOH/water, 1:1:1, 3 mL) was stirred at ambient temperature for 30 minutes. The reaction mixture was made acidic, diluted with water, and extracted with EtOAc (5 mL×3). The combined organic layers were washed with saturated brine, dried over MgSO₄, filtered, and concentrated under reduced pressure. The residue was purified by flash column chromatography (silica gel, 30% EtOAc in hexane). (R/S)-[6-(2′-Butoxy-5′-methyl-biphenyl-4-ylmethoxy)-1,2,3,4-tetrahydro-naphthalen-1-yl]-acetic acid (1) was obtained as a colorless oil (51 mg, 60%). LC-MS ESI (neg.) m/e: 457.3 (M−H). ¹H NMR (500 MHz) (CDCl₃) δ 7.59 (d, 2H); 7.46 (d, 2H); 7.14 (m, 3H); 6.90 (m, 2H); 6.77 (s, 1H); 5.09 (s, 2H); 3.96 (t, 2H); 3.37 (m, 1H); 2.80 (m, 3H); 2.63 (m, 1H); 2.36 (s, 3H); 1.79 (m, 6H); 1.43 (m, 2H); 0.94 (t, 3H).

### Example 2

Compound 2 was prepared from compound 1.3 according to the methods described in Example 1.

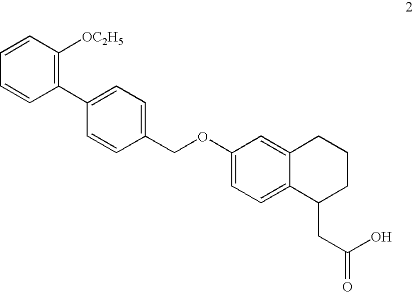

[6-(2′-Ethoxy-biphenyl-4-ylmethoxy)-1,2,3,4-tetrahydro-naphthalen-1-yl]-acetic acid (2): LC-MS ESI (neg.) m/e: 415.1 (M−H). ¹H NMR (400 MHz) (DMSO) δ 7.54 (d, 2H); 7.46 (d, 2H); 7.35 (d, 2H); 7.15 (m, 2H); 7.03 (t, 1H); 6.75 (d, 1H); 6.69 (s, 1H); 5.07 (s, 2H); 4.09 (q, 2H); 3.12 (m, 1H); 2.67 (m, 2H); 2.23 (m, 1H); 2.03 (s, 1H); 1.75 (m, 2H); 1.63 (m, 2H); 0.94 (t, 3H).

### Example 3

Compound 3 was prepared from compound 1.4 according to the methods described in Example 1.

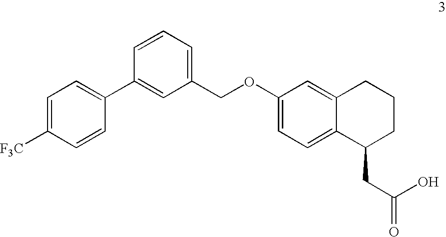

[(S)-6(4′-Trifluoromethyl-biphenyl-3-ylmethoxy)-1,2,3,4-tetrahydro-naphthalen-1-yl]-acetic acid(3): LC-MS ESI (pos.) m/e: 463.1 (M+Na). ¹H NMR (500 MHz) (CDCl₃) δ 7.74 (s, 3H); 7.68 (s, 1H); 7.58 (d, 1H); 7.50 (m, 1H); 7.14 (d, 1H); 6.84 (d, 1H); 6.67 (s, 1H): 5.12 (s, 2H); 3.34 (m, 1H); 2.78 (m, 3H); 2.60 (m, 1H); 1.96 (m, 1H); 1.80 (m, 3H).

### Examples 4-5

Compounds 4-5 were prepared from compound 1.4 according to the methods described in Example 1.

### Example 6

Compound 6 was prepared from compound 1.5 according to the methods described in Example 1.

### Examples 7-10

Compounds 7-10 were prepared from compound 1.3 according to the methods described in Example 1.

### Examples 11-15

Compounds 11-15 were prepared from 5-hydroxy-1-indanone according to the methods described in Example 1.

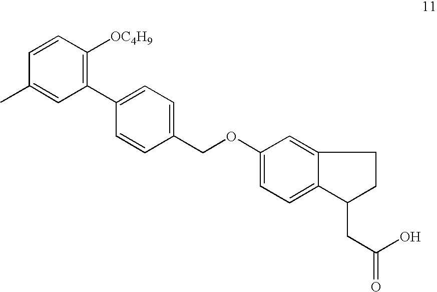

(R/S)-[5-(2′-Butoxy-5′-methyl-biphenyl-4-ylmethoxy)-indan-1-yl]-acetic acid (11): LC-MS ESI (neg.) m/e: 433.2 (M−H). ¹H NMR (500 MHz) CDCl₃) δ 7.59 (d, 2H); 7.50 (d, 2H); 7.16 (m, 3H); 6.93 (m, 3H); 5.11 (s, 2H); 3.97 (t, 2H); 3.59 (m, 1H); 2,87 (m, 3H); 2.52 (m, 2H); 2.36 (s, 3H); 1.85 (m, 1H); 1.75 (m, 2H); 1.43 (m, 2H); 0.94 (t, 3H).

### Examples 16-19

Compounds 16-19 were prepared from 6-hydroxy-1-tetralone according to the methods described in Example 1.

### Examples 20-21

Compounds 20-21 were prepared from 6-methoxy-2-tetralone by a sequence substantially similar to that described for Example 1.

### Examples 22-24

Compounds 22-24 were prepared from 6-(4-methoxybenzyloxy)-2-methyl-3,4-dihydronaphthalen-1(2H)-one (22.1) by a sequence similar to that described for Example 1.

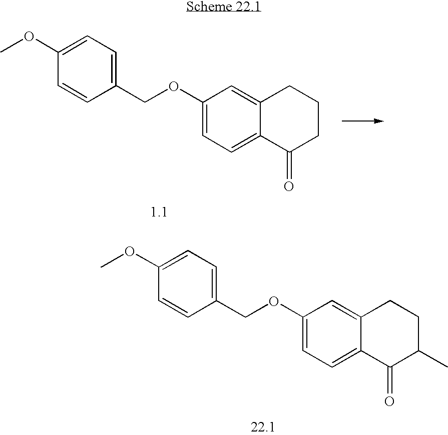

6-(4-Methoxybenzyloxy)-2-methyl-3,4-dihydronaphthalen-1(2H)-one (22.1). 6-(4-Methoxybenzyloxy)-3,4-dihydronaphthalen-1(2H)-one (1.1) (2.82 g, 10 mmol) was dissolved in anhydrous THF and cooled to −78° C. LDA (1 M in THF, 12 mL) was slowly added dropwise to the above solution. The resulting mixture was stirred at −78° C. for 30 minutes before MeI (7.1 g, 50 mmol) was added dropwise. The resulting mixture was warmed to room temperature slowly and stirred for another 12 hours at ambient temperature. The reaction mixture was diluted with water (200 mL) and extracted with EtOAc (50 mL×3). The combined organic layers were washed with saturated brine, dried over MgSO₄, filtered, and concentrated under reduced pressure. The residue was purified using flash chromatography with EtOAc and hexane (1:3) to yield 22.1 (1.57 g, 53%). MS ESI (pos.) m/e: 297 (M+1)⁺.

### Examples 25-28

Compounds 25-28 were prepared from 5-(4-methoxy-benzyloxy)-2-methyl-indan-1-one using a procedure similar to that described for Example 1. 5-(4-Methoxy-benzyloxy)-2-methyl-indan-1-one was prepared from 5-(4-methoxy-benzyloxy)-indan-1-one using a procedure analogous to that described for 22.1 from 1.1.

### Example 29

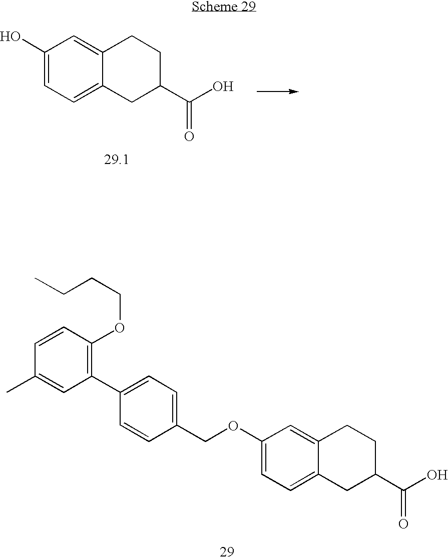

6-Hydroxy-1,2,3,4-tetrahydronaphthalene-2-carboxylic acid (29.1). This compound was prepared using the method described in U.S. Pat. No. 6,703,082.

(R/S)-6-(2′-Butoxy-5′-methyl-biphenyl-4-ylmethoxy)-1,2,3,4-tetrahydro-naphthalene-2-carboxylic acid (29). A mixture of 29.1 (50 mg, 0.26 mmol), 4′-bromomethyl-2-butoxy-5-methyl-biphenyl (D) (183 mg, 0.55 mg) and Cs₂CO₃ (212 mg, 0.65 mmol) was stirred at room temperature for 2 days. LiOH (27 mg, 0.65 mmol) was added along with water (0.5 mL), and the reaction mixture was further stirred overnight. The reaction mixture was acidified with dilute HCl, diluted with water, and extracted with EtOAc, and the combined organic layers were washed with water. The residue obtained after concentration was purified by reverse phase HPLC to yield 29 (5 mg). LC-MS ESI (neg.) m/e: 443 (M−H). ¹H NMR (500 MHz) CDCl₃) δ 7.54 (d, 2H); 7.45 (d, 2H); 7.56-7.4, (m, 3H); 6.9-6.7 (m, 3H); 5.1 (s, 2H); 3.98 (t, 2H); 3.1-2.7 (m, 5H); 2.4 (s, 3H); 2.3 (m, 1H); 1.9 (m, 1H); 1.7 (m, 2H); 1.4 (m, 2H); 0.9 (t, 3H).

### Example 30

Compound 30 was prepared from compound 29.1 and 1-(2-bromoethoxy)-3-(trifluoromethyl)benzene (obtained from Aldrich, Milwaukee, Wis.) according to the method described in Example 29.

### Example 31

Compound 31 was prepared from compound 29.1 and compound A according to the method described in Example 29.

### Example 32

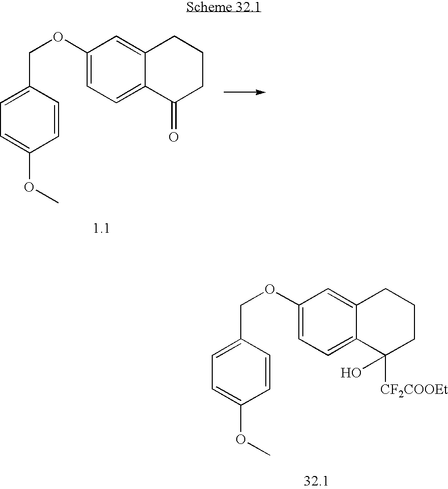

Difluoro-[1-hydroxy-6-(4-methoxy-benzyloxy)-1,2,3,4-tetrahydro-naphthalen-1-yl]-acetic acid ethyl ester (32.1). Zinc (1.84 g, 28.36 mmol), a crystal of iodine, and THF (20 mL) were added to a flask charged with nitrogen. The mixture was heated to reflux. Then, a solution of 1.1 (4.0 g, 14.18 mmol) was added followed by ethyl bromodifluoroacetate (4.61 g, 22.69 mmol). The mixture was heated for another 3 hours. The reaction mixture was poured into water and extracted with EtOAc (2×20 mL). The combined extracts were washed with water followed by brine. The product was purified by flash chromatography to give 32.1 as a pale yellow solid (3.5 g, 38%). MS ESI (pos.) m/e: 429.2 (M+23).

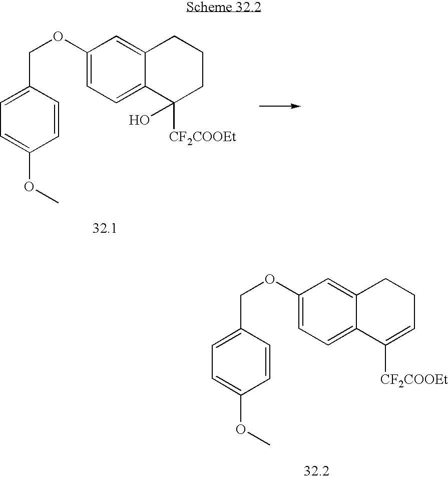

Difluoro-[6-(4-methoxy-benzyloxy)-3,4-dihydro-naphthalen-1-yl]-acetic acid ethyl ester (32.2). Martin's reagent (bis[α,α-bis(trifluoromethyl)benzene-methanolato]diphenylsulfur) (4 g, 5.96 mmol) in DCM (10 mL) was added to a solution of 32.1 (2 g, 4.93 mmol) in DCM (40 mL)under nitrogen atmosphere. The resulting mixture was stirred overnight. The reaction mixture was concentrated using rotary evaporation, and the resulting residue was purified using flash chromatography yielding 32.2 (1.6 g, 84%). MS ESI (pos.) m/e: 411.1 (M+23).

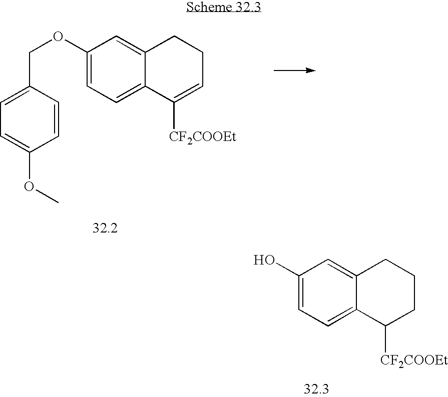

Difluoro-(6-hydroxy-1,2,3,4-tetrahydro-naphthalen-1-yl)-acetic acid ethyl ester (32.3). A flask was charged with 32.2 (1.55 g, 3.99 mmol), 10% Pd/C (400 mg), and EtOH (40 mL). A hydrogen filled balloon was attached to the reaction vessel, and the vessel was evacuated and backfilled with hydrogen three times. The reaction was stirred vigorously under a hydrogen atmosphere overnight. The reaction mixture was filtered through a plug of Celite® to remove the Pd/C, and the resulting solution was purified by flash chromatography yielding desired product 32.3 (1.0 g, 94%). MS ESI (pos.) m/e: 271.1 (M+H).

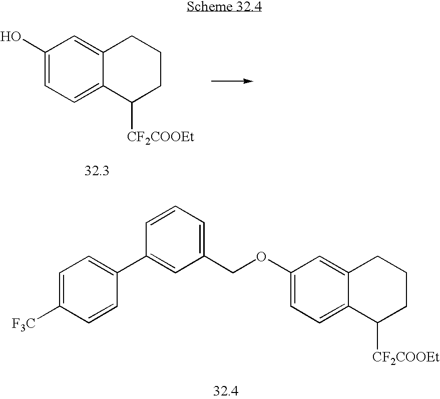

Difluoro-[6-(4′-trifluoromethyl-biphenyl-3-ylmethoxy)-1,2,3,4-tetrahydro-naphthalen-1-yl]-acetic acid ethyl ester (32.4). A flask was charged with 32.3 (0.10 g, 0.37 mmol), E (0.117 g, 0.37 mmol), Cs₂CO₃ (0.181 g, 0.56 mmol), and DMF (3 mL). The solution was stirred at room temperature for 5 hours. The reaction mixture was then poured into water and extracted with EtOAc (2×20 mL). The combined extracts were washed with water and then with brine. The product was purified by flash chromatography to give 32.4 (0.14 g, 80%). MS ESI (pos.) m/e: 505.1 (M−H).

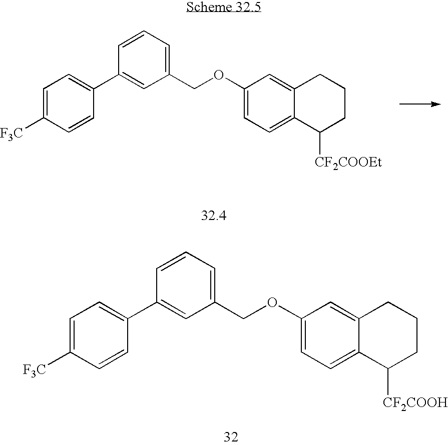

Difluoro-[6-(4′-trifluoromethyl-biphenyl-3-ylmethoxy)-1,2,3,4-tetrahydro-naphthalen-1-yl]-acetic acid (32). To a solution of 32.4 (0.080 g, 0.159 mmol) in MeOH (3 mL), was added 1 N NaOH (3 mL, 3.0 mmol). The reaction mixture was stirred at room temperature overnight. The reaction mixture was then partitioned between EtOAc and 1N HCl, and extracted with EtOAc (2×5 mL). The combined extracts were concentrated, and the resulting residue was purified by flash chromatography to yield 32 (51 mg, 68%). ¹H NMR (400 MHz) (DMSO) δ 8.01 (m, 2H); 7.91 (m, 3H); 7.71 (m, 1H); 7.55 (m, 2H); 7.16 (m, 1H); 6.84 (m, 2H); 5.17 (s, 2H); 3.52 (m, 1H); 2.69 (m, 2H); 1.85 (m, 3H); 1.61 (m, 1H). MS ESI (neg.) m/e: 475.1 (M−H).

### Example 33

Compound 33 was prepared from compound 32.3 and compound C according to the method described in Example 32.

### Example 34

This example illustrates the preparation 2-(7-((4-methyl-2-p-tolylthiazol-5-yl)methoxy)-3-methylene-3,4-dihydro-2H-chromen-4-yl)acetic acid (34).

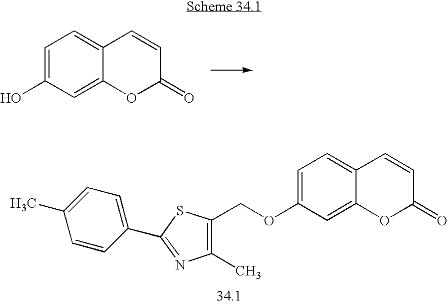

7-((4-Methyl-2-p-tolylthiazol-5-yl)methoxy)-2H-chromen-2-one (34.1). A mixture of 7-hydroxycoumarin (3.24 g, 20 mmol) and C (5.5 g, 20 mmol) was dissolved in DMF (30 mL). Cs₂CO₃ (14.3 g, 44 mmol) was added in portions to the solution at room temperature. The mixture was then stirred at 45° C. overnight. After cooling, the mixture was treated with water (100 mL) and brought to a pH of about 6 with 3 N HCl (about 30 mL). The solid was collected by filtration, washed with water, and dried providing 7-((4-methyl-2-p-tolylthiazol-5-yl)methoxy)-2H-chromen-2-one (34.1) (7 g, 95% yield). MS ESI (pos.) m/e: 364.1 (M+H). ¹H NMR (400 MHz) (DMSO-d6) δ 8.02 (d, 1H); 7.81 (d, 2H); 7.67 (d, 1H); 7.31 (d, 2H); 7.16 (d, 1H); 7.05 (dd, 1H); 6.33 (d, 1H); 5.45 (s, 2H); 2.47 (s, 3H); 2.37 (s, 3H).

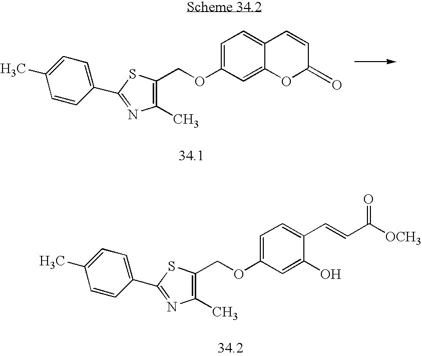

(E)-Methyl 3-(2-hydroxy-4-((4-methyl-2-p-tolylthiazol-5-yl)methoxy)phenyl)acrylate (34.2). Sodium (1 g, 43 mmol) was added in small pieces to dry MeOH (60 mL) at room temperature. 34.1 (4 g, 10 mmol) was then added to the mixture in small portions. The mixture was stirred at 65° C. for 12 hours. After cooling to 0° C., the mixture was neutralized using 3 N HCl (14.3 mL), and diluted with water (200 mL). The solid was collected by filtration, washed with water, and dried providing (E)-methyl 3-(2-hydroxy-4-((4-methyl-2-p-tolythiazol-5-yl)methoxy)phenyl)acrylate (34.2) (2.7 g, 98% yield). LC-MS ESI (pos.) m/e: 396.1 (M+H).

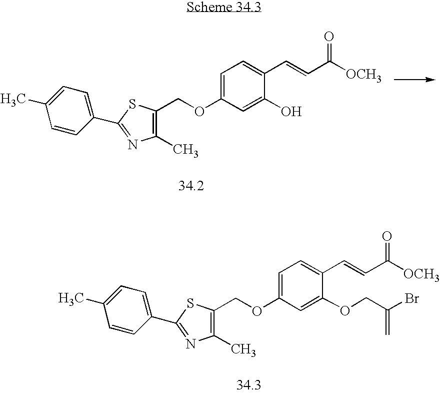

(E)-Methyl 3-(2-(2-bromoallyloxy)-4-((4-methyl-2-p-tolylthiazol-5-yl)methoxy)phenyl)acrylate (34.3). A mixture of 34.2 (395 mg, 1 mmol) and 2,3-dibromopropene (0.135 mL, 1.3 mmol) in DMF (2 mL) was treated with Cs₂CO₃ (456 mg, 1.4 mmol) at room temperature. The resulting mixture was then stirred at 45° C. for 8 hours. After cooling, the mixture was treated with water (10 mL) and EtOAc (20 mL). The organic layer was separated, washed twice with brine, dried with MgSO₄, and concentrated under vacuum. The crude product was purified by flash column chromatography providing (E)-methyl 3-(2-(2-bromoallyloxy)-4-((4-methyl-2-p-tolylthiazol-5-yl)methoxy)phenyl)acrylate (34.3) (500 mg, 95% yield). MS ESI (pos.) m/e: 514.1 (M+H).

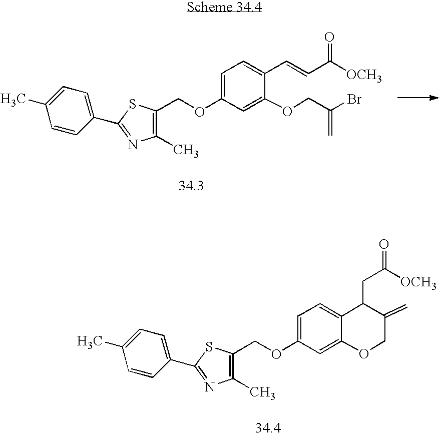

[6-(4-Methyl-2-p-tolyl-thiazol-5-ylmethoxy)-2-oxo-1,2,3,4-tetrahydro-naphthalen-1-yl]-acetic acid methyl ester (34.4). A solution of 34.3 (255 mg, 0.5 mmol) in toluene (10 mL) was degassed by bubbling nitrogen into the solution for 20 minutes and heated to 95° C. A solution of Bu₃SnH (175 mg, 0.6 mmol) and AIBN (33 mg, 0.2 mmol) in toluene (6 mL) was degassed using the same procedure described above, and the degassed mixture was then added by syringe pump over 1.2 hours to the solution of 34.3. After addition was complete, the reaction mixture was cooled and concentrated, and the crude product was purified by reverse phase HPLC to give [6-(4-methyl-2-p-tolyl-thiazol-5-ylmethoxy)-2-oxo-1 ,2,3,4-tetrahydro-naphthalen-1-yl]-acetic acid methyl ester (34.4) (trifluoroacetic acid salt) (130 mg, 60% yield). LCMS ESI (pos.) m/e: 436.1 (M+1)⁺, ¹H NMR (400 MHz) CDCl₃) δ 7.81 (d, 2H); 7.26 (d, 2H); 7.05 (d, 1H); 6.58 (dd, 1H); 6.48 (d, 1H); 6.20 (bs, 1H); 5.20 (s, 1H); 5.16 (s, 1H); 5.14 (s, 2H); 4.64 (d, 1H); 4.50 (d, 1H); 3.91 (dd, 1H); 3.71 (s, 3H); 2.8 (dd, 1H); 2.68 (dd, 1H); 2.52 (s, 3H); 2.41 (s, 3H).

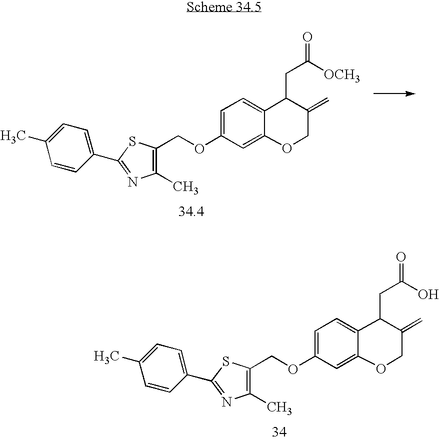

[6-(4-Methyl-2-p-tolyl-thiazol-5-ylmethoxy)-2-oxo-1,2,3,4-tetrahydro-naphthalen-1-yl]-acetic acid (34). A solution of 34.4 (66 mg, 0.13 mmol) in THF (2 mL), MeOH (2 mL), and water (1 mL) was treated with LiOH monohydrate (27 mg, 0.64 mmol). The resulting mixture was stirred at room temperature for 2 days. The organic solvent was removed by blowing air over the mixture. The aqueous layer was acidified by adding 3 N HCl, and the resulting mixture was then extracted with DCM. The organic layer was separated, washed twice with brine, dried with MgSO₄, and concentrated under vacuum. The crude product was purified by flash column chromatography providing [6-(4-methyl-2-p-tolyl-thiazol-5-ylmethoxy)-2-oxo-1,2,3,4-tetrahydro-naphthalen-1-yl]-acetic acid (34) (60 mg, 95%). LCMS ESI (pos.) m/e: 421.1 (M+1)⁺. ¹H NMR (400 MHz) CDCl₃) δ 7.81 (d, 2H); 7.24 (d, 2H); 7.07 (d, 1H); 6.59 (dd, 1H); 6.50 (d, 1H); 6.20 (bs, 1H); 5.25 (s, 1H); 5.19 (s, 1H); 5.14 (s, 2H); 4.64 (d, 1H); 4.51 (d, 1H); 3.92 (dd, 1H); 2.88 (dd, 1H); 2.72 (dd, 1H); 2.51 (s, 3H); 2.41 (s, 3H).

### Examples 35-37

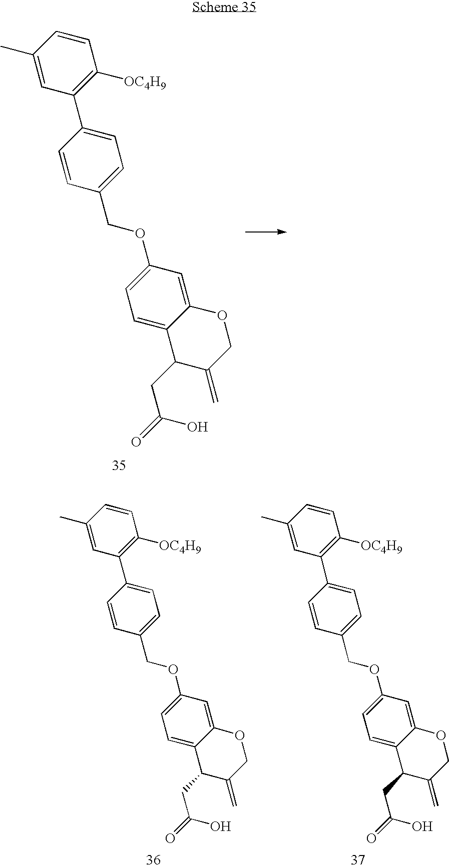

Compound 35 was prepared in a manner analogous to that of compound 34 using D instead of C.

[(S)-7-(2′-Butoxy-5′-methyl-biphenyl-4-ylmethoxy)-3-methylene-chroman-4-yl]-acetic acid (36) and [(R)-7-(2′-Butoxy-5′-methyl-biphenyl-4-ylmethoxy)-3-methylene-chroman-4-yl]-acetic acid (37) were obtained by separating the mixture of enantiomers 35 using a semi-preparatory AD-H column (7% IPA in hexanes). ¹H NMR (500 MHz) CDCl₃) δ 7.58 (d, 2H); 7.45 (d, 2H); 7.16 (d, 1H); 7.12 (d, 1H); 7.06 (d, 1H); 6.90 (d, 1H); 6.65 (d, 1H); 6.52 (d, 1H); 5.25 (s, 1H); 5.19 (s, 1H); 5.11 (s, 2H); 4.65 (d, 1H); 4.50 (d, 1H); 3.95 (m, 3H); 2.88 (q, 1H); 2.75 (q, 1H); 2.35 (s, 3H); 1.70 (m, 2H); 1.40 (m, 2H); 0.94 (t, 3H).

### Example 38

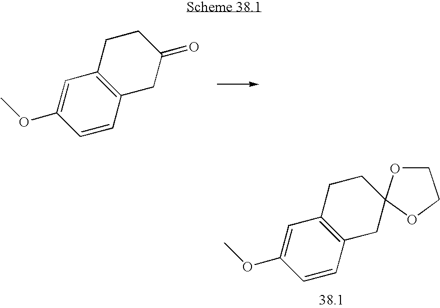

6-Methoxy-3,4-dihydronaphthalen-2(1H)-one 2-ethyleneketal (38.1). A mixture of 6-methoxy-3,4-dihydronaphthalen-2(1H)-one (14.2 mmol), trimethyl orthoacetate (22.7 mmol), toluenesulfonic acid monohydrate (0.43 mmol), and ethylene glycol (85 mmol) was stirred at room temperature for 24 hours. The reaction mixture was poured into a sodium bicarbonate solution and extracted with EtOAc (400 mL). The organic phase was washed with brine and dried over anhydrous Na₂SO₄. After removal of solvent, the residue was purified using column chromatography (silica gel, 1:4 EtOAc/hexane), to obtain compound 38.1 in 85% yield. MS API-ES m/e: 221 (M+H). ¹H NMR (500 MHz) (DMSO-d₆) δ 6.96 (d, 1H); 6.70 (m, 2H); 3.98 (m, 4H); 3.73 (m, 3H); 2.85 (m, 4H); 1.85 (m, 2H).

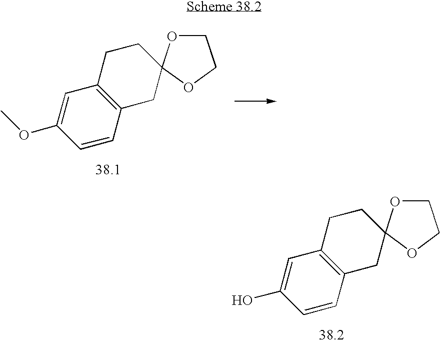

6-Hydroxy-3,4-dihydronaphthalen-2(1H)-one 2-ethyleneketal (38.2). A mixture of compound 38.1 (10.5 mmol) and sodium thiomethoxide (35 mmol) in DMF (30 mL) was stirred at 135° C. for 6 hours. The reaction mixture was cooled to room temperature and diluted with EtOAc (300 mL). It was then washed with brine and dried over anhydrous Na₂SO₄. After removal of solvent, it was purified with column chromatography (silica gel, 1:2 EtOAc/hexane), obtaining compound 38.2 in 43% yield.

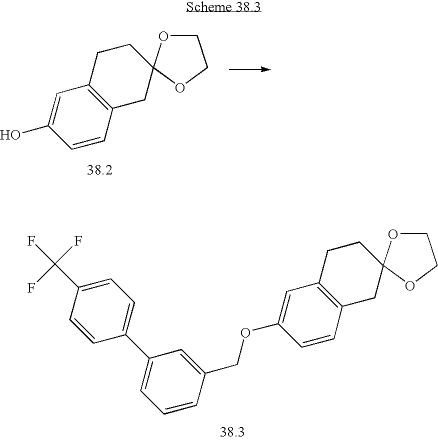

6-(3-(4-Trifluoromethylphenyl)benzyloxy-3,4-dihydronaphthalen-2(1H)-one 2-ethylene ketal (38.3). A mixture of compound 38.2 (6.5 mmol), 3-(4-trifluoromethylphenyl)benzyl bromide (E) (7.2 mmol) and K₂CO₃ (13.1 mmol) in DMF (23 mL) was stirred at room temperature overnight. The mixture was then diluted with EtOAc (200 mL), washed with brine, and dried over anhydrous Na₂SO₄. After removal of solvent, the residue was purified with column chromatography (silica gel, 1:3 EtOAc/hexane), and compound 38.3 was obtained as a white solid, in 100% yield.

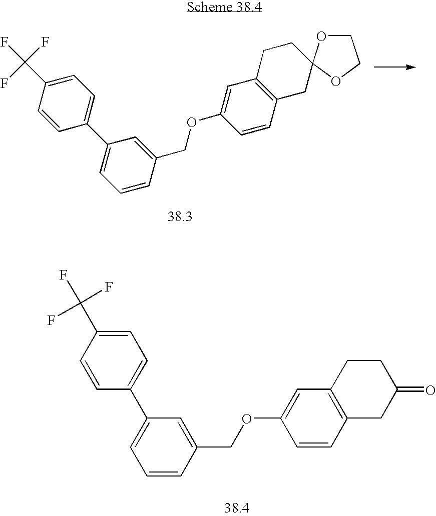

6-(3-(4-Trifluoromethylphenyl)benzyloxy)-3,4-dihydronaphthalen-2(1H)-one (38.4). A mixture of compound 38.3 (6.4 mmol) and toluenesulfonic acid monohydrate (0.95 mmol) in water (2 mL) and acetone (50 mL) was refluxed overnight. The reaction mixture was neutralized with a sodium bicarbonate solution, and the solvent was removed under reduced pressure. The residue was diluted with EtOAc (200 mL), washed with brine, and dried over anhydrous Na₂SO₄. After removal of solvent, the residue was purified with column chromatography (silica gel, 1:3 EtOAc/hexane), and compound 38.4, was obtained as a white solid in 88% yield.

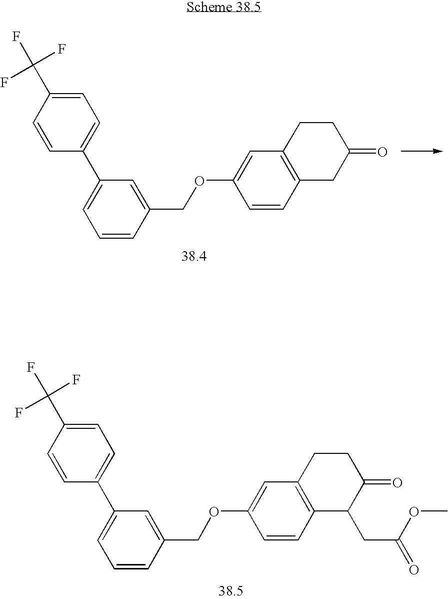

Methyl 2-(6-(3-(4-trifluoromethylphenyl)benzyloxy)-2-oxo-1,2,3,4-tetrahydronaphthalen-1-yl)acetate (38.5). At −65° C., LDA (0.56 mmol) 2.0 M in THF) was added dropwise to a solution of compound 38.4 (0.56 mmol) in THF (7 mL). The resulting mixture was stirred at the same temperature for 20 minutes. Methyl bromoacetate (0.56 mmol) was then added, and the reaction was warmed to 0° C. over 2.5 hours. The reaction mixture was then poured into cold water and extracted with EtOAc (200 mL). The organic phase was washed with brine and dried over anhydrous Na₂SO₄. After removal of solvent, the crude product was purified using column chromatography (silica gel, 1:3 EtOAc/hexane) providing compound 38.5 in 22% yield.

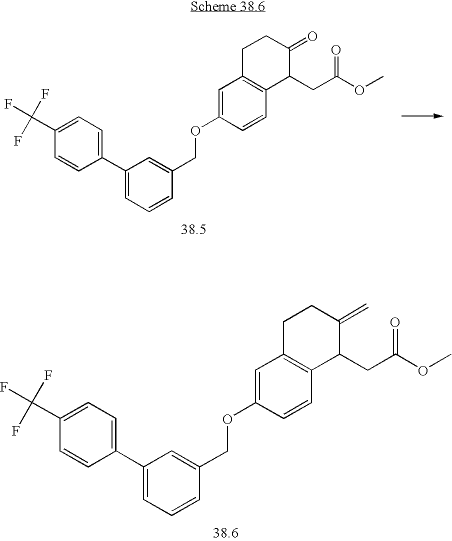

Methyl 2-(6-(3-(4-trifluoromethylphenyl)benzyloxy)-2-methylene-1,2,3,4-tetrahydronaphthalen-1-yl)acetate (38.6). Under a nitrogen atmosphere, TiCl₄ (0.14 mmol) was added dropwise to a mixture of activated zinc powder (0.64 mmol) and CH₂Br₂ (0.21 mmol) in THF (2 mL). After the mixture was stirred at room temperature for 16 minutes, a solution of compound 38.5 (0.11 mmol) in THF (1 mL) was added. The reaction was then stirred at room temperature overnight before it was quenched with water. The resulting mixture was diluted with EtOAc (200 mL), washed with brine, and dried over anhydrous Na₂SO₄. After removal of solvent, the crude product was purified using chromatography (silica gel, 1:2 EtOAc/hexane) providing compound 38.6 as a white solid in 18% yield.

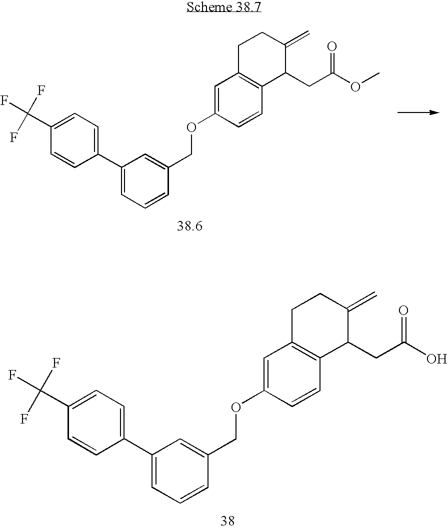

2-(6-(3-(4-Trifluoromethylphenyl)benzyloxy)-2-methylene-1,2,3,4-tetrahydronaphthalen-1-yl)acetic acid (38). A mixture of compound 38.6 (0.019 mmol) and NaOH (0.12 mmol) in water (0.5 mL) and EtOH (2 mL) was stirred at room temperature overnight. EtOH was removed, and the reaction mixture was acidified with 1 N HCl to pH 3-5. EtOAc (70 mL) was added, and the organic layer was washed with brine and dried over anhydrous Na₂SO₄. After removal of solvent, the residue obtained was purified using chromatography (silica gel, 1:2 EtOAc/hexane) providing compound 38 in 82% yield. MS API-ES m/e: 451 (M−H). ¹H NMR (500 MHz) (DMSO-d₆) δ 12.2 (s, 1H); 7.95 (m, 2H); 7.84-7.87 (m, 3H); 7.72 (d, 1H, J=7 Hz); 7.54-7.57 (m, 2H); 7.14 (d, 1H, J=8.5 Hz); 6.87 (d, 1H, J=8.5 Hz); 6.82 (s, 1H); 5.18 (s, 2H); 4.89 (s, 1H); 4.86 (s, 1H); 3.79 (m, 1H); 2.90 (m, 1H); 2.65-2.75 (m, 1H); 2.60-2.64 (m, 1H); 2.45-2.50 (m, 2H); 2.38 (s, 1H).

### Example 39

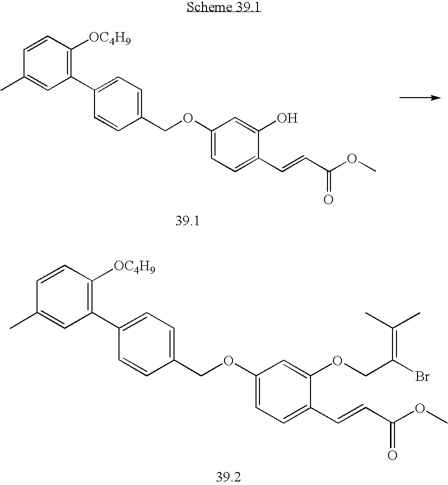

Compound 39.1 was prepared in a manner analogous to that of 34.2 using D in place of C.

(E)-3-[2-(1-Bromo-2-methyl-propenyloxy)-4-(2′-butoxy-5′methyl-biphenyl-4-ylmethoxy)-phenyl]-acrylic acid methyl ester (39.2). A mixture of 39.1 (446 mg, 1 mmol), PPh₃ (525 mg, 2 mmol), DIAD (222 mg, 1.1 mmol), and 1-hydroxy-2-bromo-3-methyl-2-butene (330 mg, 2 mmol) in anhydrous THF (5 mL) under N₂ atmosphere was stirred in a sealed tube overnight at ambient temperature. The reaction mixture was diluted with water and extracted with EtOAc (5 mL×3). The combined organic layers were washed with saturated brine, dried over MgSO₄, filtered, and concentrated under reduced pressure. The residue was purified by flash column chromatography (silica gel, DCM). (E)-3-[2-(1-Bromo-2-methyl-propenyloxy)-4-(2′-butoxy-5′-methyl-biphenyl-4-ylmethoxy)-phenyl]-acrylic acid methyl ester 39.2 was obtained as a white solid (432 mg, 73%). LC-MS ESI (pos.) m/e: 594.1 (M+H)⁺.

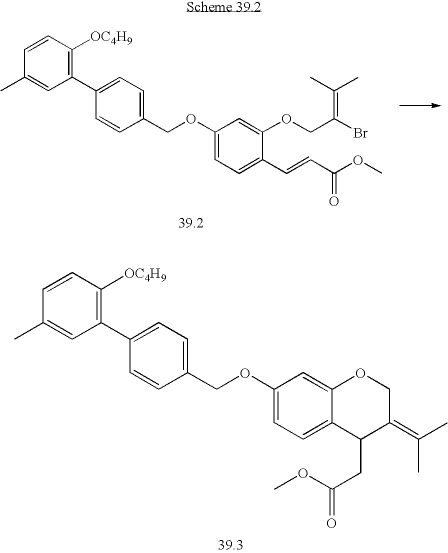

(R/S)-[6-(2′-Butoxy-5′-methyl-biphenyl-4-ylmethoxy)-2-isopropylidene-1,2,3,4-tetrahydronaphthalen-1-yl]-acetic acid methyl ester (39.3). A solution of 39.2 (500 mg, 0.84 mmol) in toluene (12 mL) was degassed with N₂ for 15 minutes. A mixture of AIBN (86 mg, 0.52 mmol) and Bu₃SnH (512 mg, 1.76 mmol) in toluene (5 mL) was added to the reaction mixture through a syringe pump over 2 hours. The resulting mixture was heated at 85° C. for 16 hours. The reaction mixture was then concentrated under reduced pressure. The residue was purified by flash column chromatography (silica gel, 25% EtOAc in hexane), and (R/S)-[6-(2′-butoxy-5′-methyl-biphenyl-4-ylmethoxy)-2-isopropylidene-1,2,3,4-tetrahydronaphthalen-1-yl]-acetic acid methyl ester (39.3) was obtained as a white solid (263 mg, 61%). LC-MS ESI (pos.) m/e: 515.2 (M+H)⁺, 537.3 (M+Na)⁺.

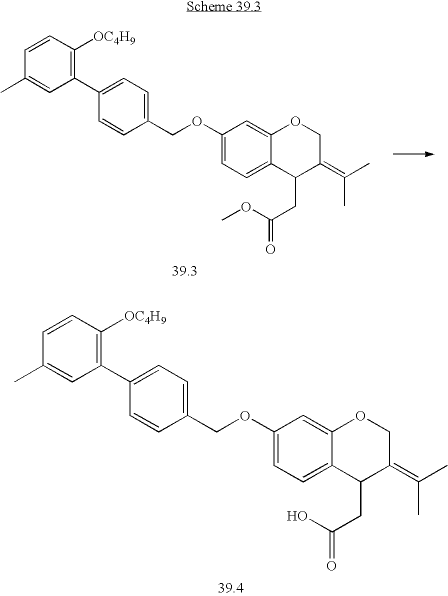

(R/S)-[6-(2′-Butoxy-5′methyl-biphenyl-4-ylmethoxy)-2-isopropylidene-1,2,3,4-tetrahydronaphthalen-1-yl]-acetic acid (39.4). Compound 39.4 was prepared from compound 39.3 according to the method described in Example 1. LC-MS ESI (neg.) m/e: 499.3 (M−H). ¹H NMR (400 MHz) (CDCl₃) δ 7.56 (d, 2H); 7.45 (d, 2H); 7.15 (s, 1H); 7.10 (m, 3H); 6.90 (d, 1H); 6.61 (d, 1H); 6.51 (s, 1H); 5.06 (s, 2H); 4.91 (d, 1H); 4.49 (d, 1H); 4.23 (m, 1H); 3.94 (m, 2H); 2.70 (m, 2H); 2.35 (s, 3H); 1.80 (s, 3H); 1.71 (s, 3H); 1.68 (m, 2H); 1.41 (m, 2H); 0.92 (m, 3H).

### Example 40

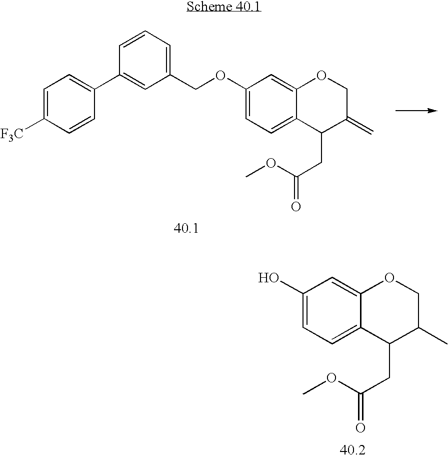

Compound 40.1 was made in a manner analogous to that of Example 34 using A instead of C.

(R/S)-(7-Hydroxy-3-methyl-chroman-4-yl)-acetic acid (40.2). Compound 40.2 was prepared from compound 40.1 according to the method described in Example 1.3. LC-MS ESI (pos.) m/e: 237.1 (M+H). ¹H NMR (400 MHz) (CDCl₃) δ 6.95 (d, 1H); 6.35 (m, 1H); 6.30 (d, 1H); 4.11 (m, 1H); 3.90 (m, 1H); 3.72 (s, 3H); 3.39 (m, 0.65H); 2.95 (m, 0.35H); 2.55 (m, 2H); 2.28 (m, 0.7H); 1.99 (m, 0.3H); 1.07 (m, 3H).

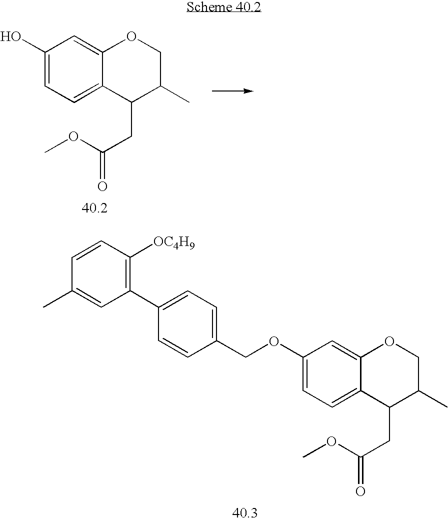

(rac)-[7-(2′-Butoxy-5′-methyl-biphenyl-4-ylmethoxy)-3-methyl-chroman-4-yl]-acetic acid methyl ester (40.3). Compound 40.3 was prepared from compound 40.2 according to the method described in Example 1.6.

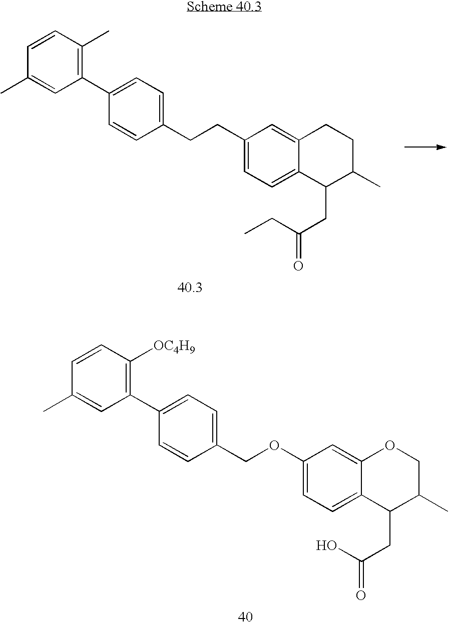

(rac)-[6-(2′-Butoxy-5′-methyl-biphenyl-4-ylmethoxy)-2-methyl-1,2,3,4-tetrahydro-naphthalen-1-yl]-acetic acid (40). Compound 40 was prepared from compound 40.3 according to the methods described in Example 1.7. LC-MS ESI (neg.) m/e: 473.2 (M−H). ¹H NMR (500 MHz) (CDCl₃) δ 7.58 (d, 2H); 7.46 (d, 2H); 7.16 (s, 1H); 7.12 (d, 1H); 7.06 (d, 1H); 6.91 (d, 1H); 6.65 (m, 1H); 6.50 (m, 1H); 5.06 (s, 2H); 4.15 (m, 1H); 3.93 (m, 3H); 3.52 (m, 2/3H); 3.02 (m, 1/3H); 2.66 (m, 2H); 2.35 (m, 4H); 1.72 (m, 2H); 1.43 (m, 2H); 1.12 (d, 1H); 1.03 (d, 2H); 0.94 (m, 3H).

### Example 41

Compound 41 was prepared using the same methodology used to prepare compound 39. Compound 41 was prepared using (Z)-2-bromobut-2-en-1-ol in place of the 1-hydroxy-2-bromo-3-methyl-2-butene used to prepare 39.2. (Z)-2-Bromobut-2-en-1-ol was prepared from (Z)-methyl 2-bromobut-2-enoate by DIBAL reduction using the procedure described by Fevig et al. (J. Am. Chem. Soc., 113: 5085-5086 (1991)).

### Example 42

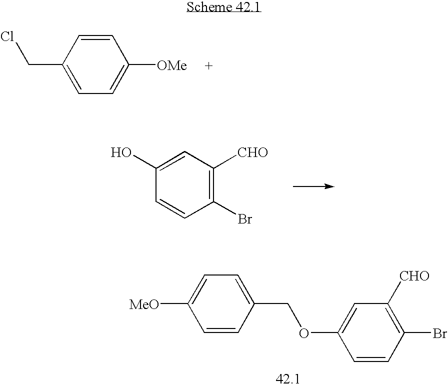

5-(4-Methoxybenzyloxy)-2-bromobenzaldehyde (42.1). A mixture of 1-(chloromethyl)-4-methoxybenzene (60 mmol) and 2-bromo-5-hydroxybenzaldehyde (50 mmol) was stirred at room temperature for 25 hours. The resulting mixture was diluted with EtOAc (800 mL) and washed with water, washed with brine, and dried over anhydrous Na₂SO₄. After removal of solvent, the residue was purified using column chromatography (silica gel, 1:4 EtOAc/hexane) providing compound 42.1 as a white solid in 88% yield. MS API-ES m/e: 321 (M+H). ¹H NMR (400 MHz) (DMSO-d₆) δ 10.16 (s, 1H); 7.70 (d, 1H, J=9 Hz); 7.37-7.42 (m, 3H); 7.27 (m, 1H); 6.94 (m, 2H); 5.10 (s, 2H); 3.76 (s, 3H).

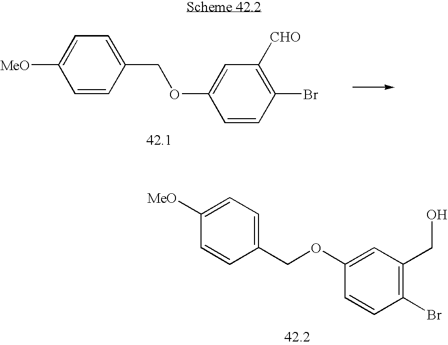

(5-(4-Methoxybenzyloxy)-2-bromophenyl)methanol (42.2). A mixture of 5-(4-methoxybenzyloxy)-2-bromobenzaldehyde (42.1, 12.7 mmol) and NaBH₄ (19 mmol) in MeOH (30 mL) was refluxed for 4 hours. MeOH was removed, and EtOAc 400 mL) was added. The organic layer was washed with an aqueous Na₂CO₃ solution, brine and dried over anhydrous Na₂SO₄. After removal of solvent, the residue obtained was purified using column chromatography (silica gel, 1:3 EtOAc/hexane) providing compound 42.2 as a white solid in 77% yield. MS API-ES m/e: 345 (M+H). ¹ NMR (400 MHz) (DMSO-d₆) δ 7.43 (d, 1H, J=8.7 Hz); 7.36 (d, 2H, J=8.6 Hz); 7.17 (m, 1H); 6.94 (d, 2H, J=8.6 Hz); 6.84 (m, 1H); 5.42 (m, 1H); 5.02 (s, 2H); 4.45 (d, 2H, J=5.5 Hz); 3.76 (s, 3H).

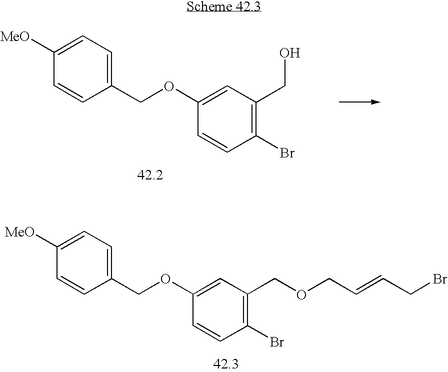

(E)-4-(4-Methoxybenzyloxy)-1-bromo-2-((4-bromobut-2-enyloxy)methyl)benzene (42.3). A mixture of (5-(4-methoxybenzyloxy)-2-bromophenyl)methanol (42.2) (5.1 mmol), (E)-1,4-dibromobut-2-ene (10.3 mmol), tetrabutylammonium iodide (0.51 mmol) and NaOH (51 mmol) in DCM (20 mL) and water (2 mL), was stirred at room temperature for 17 hours. The reaction mixture was diluted with EtOAc, the layers were separated, and the organic layer was washed with water, washed with brine, and dried over anhydrous Na₂SO₄. After removal of solvent, the residue was purified using column chromatography (silica gel, 1:6 EtOAc/hexane) providing compound 42.3 as a white solid in 83% yield.

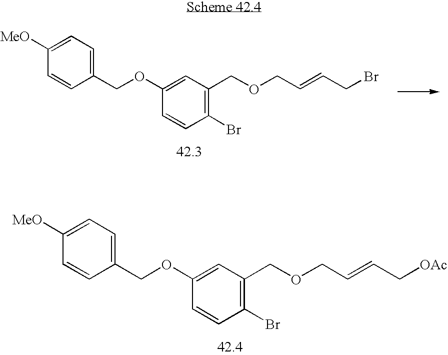

(E)-4-(5-(4-Methoxybenzyloxy)-2-bromobenzyloxy)but-2-enyl acetate (42.4). A mixture of (E)-4-(4-methoxybenzyloxy)-1-bromo-2-((4-bromobut-2-enyloxy)methyl)benzene (42.3) (3.9 mmol) and sodium acetate (6.2 mmol) in DMF was stirred at 85° C. for 2 hours. The reaction mixture was diluted with EtOAc, the layers were separated, and the organic layer was washed with water, washed with brine, and dried over anhydrous Na₂SO₄. After removal of the solvent, the residue was purified using column chromatography (silica gel, 1:4 EtOAc/hexane) providing compound 42.4 as a white solid in 87% yield.

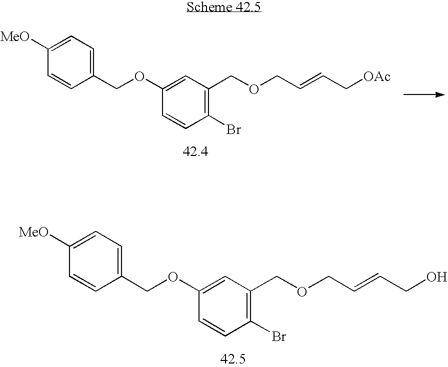

(E)-4-(5-(4-Methoxybenzyloxy)-2-bromobenzyloxy)but-2-en-1-ol (42.5). A mixture of (E)-4-(5-(4-methoxybenzyloxy)-2-bromobenzyloxy)but-2-enyl acetate (42.5) (3.3 mmol) and NaOH (12 mmol) in water (5 mL) and EtOH (25 mL) was stirred at room temperature for 19 hours. EtOH was removed, and EtOAc (30 mL) was added. The layers were separated, and the organic layer was washed with water, washed with brine, and dried over anhydrous Na₂SO₄. After removal of solvent, crude product (42.5) was obtained in 100% yield.

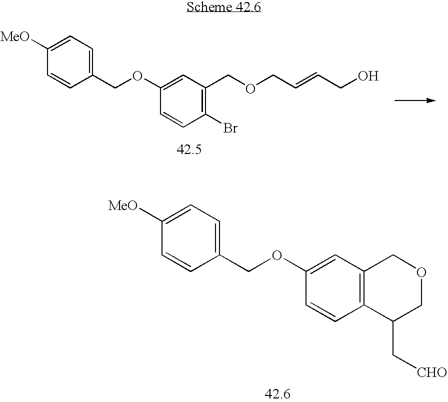

2-(7-(4-Methoxybenzyloxy)-3,4-dihydro-1H-isochromen-4-yl)acetaldehyde (42.6). A mixture of (E)-4-(5-(4-methoxybenzyloxy)-2-bromobenzyloxy)but-2-en-1-ol (42.5) (3.1 mmol), palladium acetate (0.92 mmol), and tri(O-tolyl)phosphine (1.8 mmol) in triethylamine (35 mL) was stirred at 85° C. for 2 hours. Triethylamine was removed, and the residue was purified using column chromatography (silica gel, 1:3 EtOAc/hexane) providing compound 42.6 as a white solid in 44% yield.

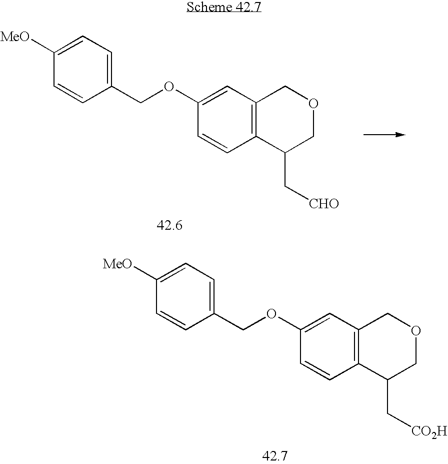

2-(7-(4-Methoxybenzyloxy)-3,4-dihydro-1H-isochromen-4-yl)acetic acid (42.7). Potassium permanganate solution (95 mg of KMnO₄ in 0.6 mL of water) (0.6 mmol) was added dropwise to a solution of 2-(7-(4-methoxybenzyloxy)-3,4-dihydro-1H-isochromen-4-yl)acetaldehyde (42.6) (0.4 mmol) in acetone (6 mL) at room temperature. As soon as the addition was complete, the reaction mixture was diluted with EtOAc, the layers were separated, and the organic layer was washed with water, washed with brine, and dried over anhydrous Na₂SO₄. Crude product (42.7) was obtained by removing the solvent.

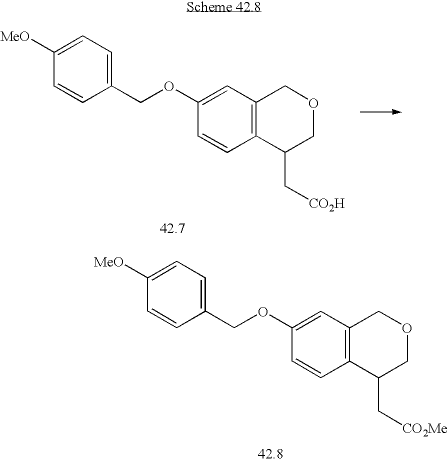

Methyl 2-(7-(4-methoxybenzyloxy)-3,4-dihydro-1H-isochromen-4-yl)acetate (42.8). Trimethylsilyidiazomethane (0.3 mmol) was added to a solution of 2-(7-(4-methoxybenzyloxy)-3,4-dihydro-1H-isochromen-4-yl)acetic acid (42.7) (0.4 mmol). The mixture was stirred at room temperature for 5 minutes. After removal of solvent, the crude product, (42.8) was obtained.

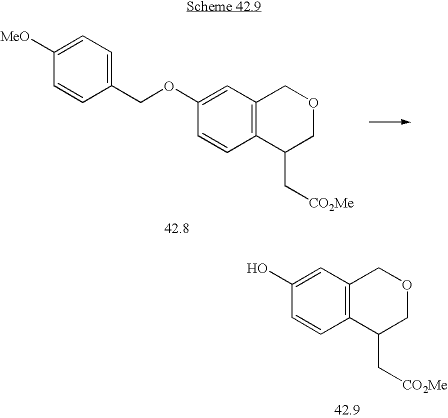

Methyl 2-(7-hydroxy-3,4-dihydro-1H-isochromen-4-yl)acetate (42.9). A mixture of methyl 2-(7-(4-methoxybenzyloxy)-3,4-dihydro-1H-isochromen-4-yl)acetate (42.8) and palladium on activated carbon in MeOH was stirred at room temperature under hydrogen atmosphere for 10 minutes. The reaction mixture was filtered through silica gel eluting with EtOAc. After removal of solvent, the crude product (42.9) was obtained.

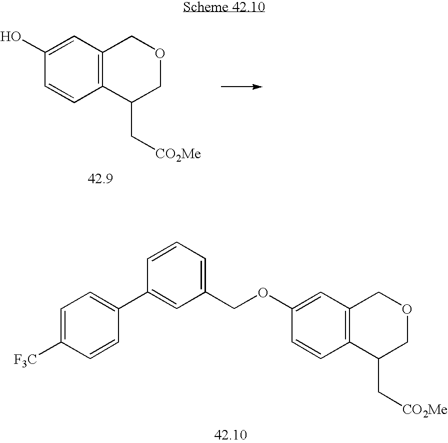

Methyl 2-(7-(3-(4-trifluoromethylphenyl)benzyloxy)-3,4-dihydro-1H-isochromen-4-yl)acetate (42.10). A mixture of methyl 2-(7-hydroxy-3,4-dihydro-1H-isochromen-4-yl)acetate (42.09), 3-(4-trifluoromethylphenyl)benzyl bromide (E) and K₂CO₃ in DMF was stirred at room temperature for 1 hour. The reaction mixture was diluted with EtOAc, and the layers were separated. The organic layer was washed with water, washed with brine, and then dried over anhydrous Na₂SO₄. After removal of solvent under reduced pressure, the residue was purified using column chromatography (silica gel, 1:2 EtOAc/hexane) providing compound 42.10 as a white solid in 33% yield.

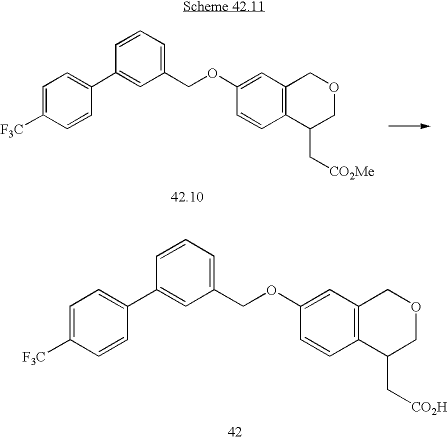

2-(7-(3-(4-Trifluoromethylphenyl)benzyloxy)-3,4-dihydro-1H-isochromen-4-yl)acetic acid (42). A mixture of methyl 2-(7-(3-(4-trifluoromethylphenyl)benzyloxy)-3,4-dihydro-1H-isochromen-4-yl)acetate (42.10) (0.011 mmol) and NaOH (0.5 mmol) in water (0.5 mL) and EtOH (2 mL) was stirred at room temperature for 2 hours. EtOH was removed, and EtOAc (70 mL) was added. The layers were separated, and the organic layer was washed with water, washed with brine, and dried over anhydrous Na₂SO₄. After removal of solvent, product (42) was obtained in 96% yield. MS API-ES m/e: 441 (M−H). ¹H NMR (500 MHz) (DMSO-d₆) δ 7.93 (m, 2H); 7.83-7.87 (m, 3H); 7.72 (m, 1H); 7.55 (m, 2H); 7.20 (d, 1H, J=8 Hz); 6.89 (d, 1H, J=8 Hz); 6.75 (s, 1H); 5.18 (s, 2H); 4.61-4.72 (m, 2H); 3.80 (s, 2H); 3.07 (m, 1H); 2.45 (m, 2H).

### Example 43

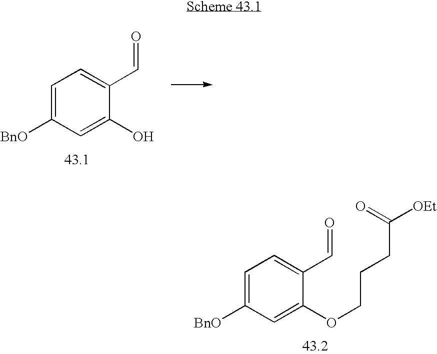

Ethyl 4-(5-(benzyloxy)-2-formylphenoxy)butanoate (43.2). 4-(benzyloxy)-2-hydroxybenzaldehyde (2.0 g, 8.76 mmol) and ethyl 4-bromobutanoate (1.38 mL, 9.64 mmol) were dissolved in 35 mL of DMF. Cs₂CO₃ (4.28 g, 13.14 mmol) was added to this solution, and the mixture was stirred for 12 hours. The mixture was then poured into 500 mL of 0.5 M HCl (aqueous) and extracted with EtOAc (3×150 mL). The organic layers were combined, washed with water, washed with brine, and then dried over MgSO₄. The organic solution was filtered and concentrated under reduced pressure. The residue was purified on silica gel with EtOAc/hexane. The fractions containing the desired material were combined and concentrated under reduced pressure providing ethyl 4-(5-(benzyloxy)-2-formylphenoxy)butanoate (43.2) (1.98 g, 5.78 mmol, 66%).

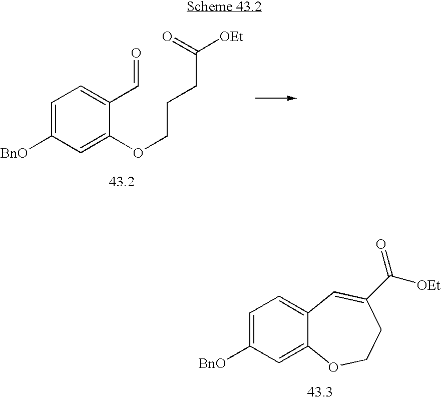

(E)-ethyl 8-(benzyloxy)-2,3-dihydrobenzo[b]oxepine-4-carboxylate (43.3). Ethyl 4-(5-(benzyloxy)-2-formylphenoxy)butanoate (43.2, 100 mg, 0.29 mmol) was combined with KOtBu (80 mg, 0.70 mmol) in a round bottom flask that was subsequently purged with nitrogen. THF (1 mL) was added to the flask, and the mixture was stirred vigorously. After 12 hours, the reaction was diluted with water (50 mL) and extracted with EtOAc (3×50 mL). The organic layers were combined, washed with brine, dried with MgSO₄, and filtered. The organic solution was concentrated under reduced pressure, and the residue obtained was purified on silica gel with 10% EtOAc/hexane. The fractions containing the desired material were combined and concentrated under reduced pressure providing (E)-ethyl 8-(benzyloxy)-2,3-dihydrobenzo[b]oxepine-4-carboxylate (43.3) 70 mg, 0.22 mmol, 75%).

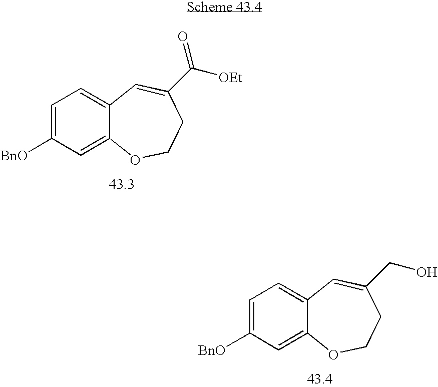

(E)-(8-(benzyloxy)-2,3-dihydrobenzo[b]oxepin-4-yl)methanol (43.4). (E)-ethyl 8-(benzyloxy)-2,3-dihydrobenzo[b]oxepine-4-carboxylate (43.3, 400 mg, 1.23 mmol) was dissolved in THF (5 mL), and the mixture was then cooled to 0° C. LiAlH₄ (1.35 mL, 1 M, 1.35 mmol) was added dropwise to the mixture, and the reaction was allowed to warm to room temperature over 2 hours. The reaction was then cooled to 0° C. and quenched with saturated NH₄Cl (aqueous). The mixture was diluted with water and extracted with EtOAc (3×50 mL). The organic layers were combined, washed with brine, dried with MgSO₄, filtered, and concentrated under reduced pressure to afford (E)-(8-(benzyloxy)-2,3-dihydrobenzo[b]oxepin-4-yl)methanol (43.4) (329 mg, 1.16 mmol, 95%).

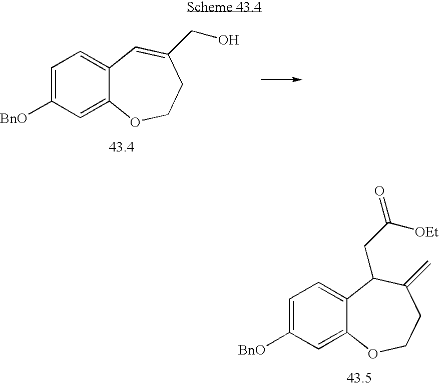

(R/S)-Ethyl 2-(8-(benzyloxy)-4-methylene-2,3,4,5-tetrahydrobenzo[b]oxepin-5-yl)acetate (43.5). (E)-(8-(benzyloxy)-2,3-dihydrobenzo[b]oxepin-4-yl)methanol (43.4) (210 mg, 0.75 mmol) was dissolved in triethylorthoacetate (10 mL) and then 5 drops of propionic acid were added to the mixture. The reaction mixture was then heated to 110° C. for 5 hours. The mixture was allowed to cool and was then poured into 100 mL EtOAc. The organic layer was washed with 2 N HCl (aqueous) (3×100 mL), dried with MgSO₄, filtered, and concentrated under reduced pressure. The residue was purified on silica gel with EtOAc/hexane. The fractions′containing the desired material were combined and concentrated under reduced pressure to afford (R/S)-ethyl 2-(8-(benzyloxy)-4-methylene-2,3,4,5-tetrahydrobenzo[b]oxepin-5-yl)acetate (43.5) (105 mg, 0.30 mmol, 40%).

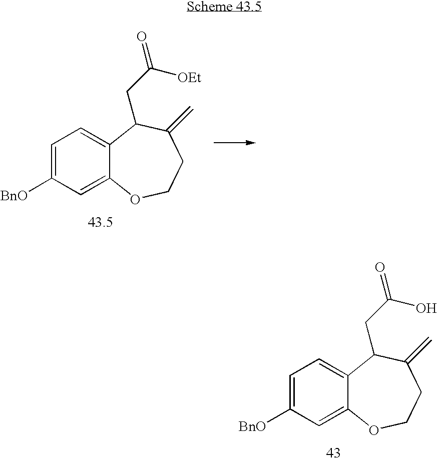

(R/S)-2-(8-(benzyloxy)-4-methylene-2,3,4,5-tetrahydrobenzo[b]oxepin-5-yl)acetic acid (43). Ethyl 2-(8-(benzyloxy)-4-methylene-2,3,4,5-tetrahydrobenzo[b]oxepin-5-yl)acetate (43.5, 21 mg, 0.059 mmol) was dissolved in THF (1 mL) and then 5 equivalents of 2 N LiOH (aqueous) was added. MeOH was then added until the mixture became homogeneous. The solution was stirred for 8 hours and was then concentrated. The residue was partitioned between DCM and 2 N HCl (aqueous.). The organic layer was dried over MgSO₄, filtered, and concentrated to afford (R/S)-2-(8-(benzyloxy)-4-methylene-2,3,4,5-tetrahydrobenzo[b]oxepin-5-yl)acetic acid (43) (19 mg, 0.058 mmol, 99%). ¹H NMR (400 MHz) (CDCl₃) δ 7.29-7.42 (m, 5H); 7.09-7.12 (m, 1H); 6.62-6.66 (m, 2H); 5.00 (s, 2H); 4.97 (s, 1H); 4.79 (s, 1H); 4.37 (dt, 1H, J=4.0, 11.7 Hz); 3.88 (t, 1H, J=7.7 Hz); 3.69 (dt, 1H, J=2.2 11.5 Hz); 2.85-3.00 (m, 3H); 2.42 (m, 1H).

### Examples 44X and 44

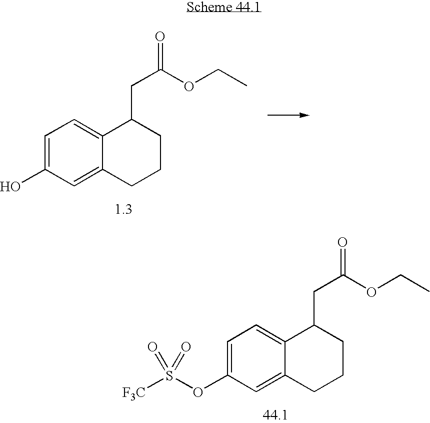

(+/−) Ethyl 2-(6-(trifluoromethylsulfonyloxy)-1,2,3,4-tetrahydronaphthalen-1-yl)acetate (44.1). A solution of ethyl 2-(6-hydroxy-1,2,3,4-tetrahydronaphthalen-1-yl)acetate 1.3 (1.46 g, 6.2 mmol) and triethylamine (1.0 mL) in DCM (20 mL) at 0° C., was treated with triflic anhydride (1.0 mL) which was slowly added by syringe. The reaction mixture was brought to room temperature over 20 hours. Solvent and excess volatile reagents were removed under reduced pressure. The product was purified using flash chromatography (0-15% EtOAc in hexane). Ethyl 2-(6-(trifluoromethylsulfonyloxy)-1,2,3,4-tetrahydronaphthalen-1-yl)acetate (44.1) was obtained as colorless oil (2.0 g, 5.5 mmol, 88%). MS ESI (pos.) m/e: 367 (M+H).

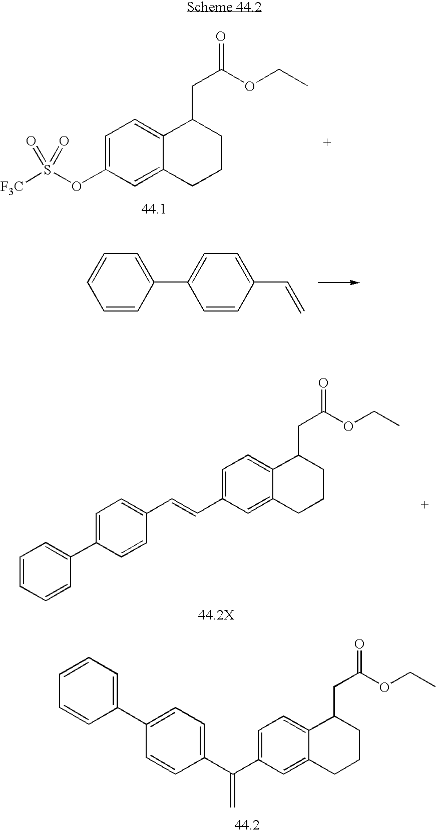

(R/S)-[6-((E)-2-Biphenyl-4-yl-vinyl)-1,2,3,4-tetrahydro-naphthalen-1-yl]-acetic acid ethyl ester (44.2X) and (R/S)-[6-(1-Biphenyl-4-yl-vinyl)-1,2,3,4-tetrahydro-naphthalen-1-yl]-acetic acid ethyl ester (44.2). To a mixture of 4-phenyl styrene (394 mg, 2.2 mmol) and ethyl 2-(6-(trifluoromethylsulfonyloxy)-1,2,3,4-tetrahydronaphthalen-1-yl)acetate (44.1) (160 mg, 0.44 mmol) in DMF (4 mL) in a 2 dram vial, was added a mixture of BINAP and palladium acetate (1.05:1.00, 50 mg) under a nitrogen atmosphere. The vial was sealed tightly, and the reaction mixture was stirred at 115° C. overnight. After the reaction was complete, the mixture was cooled to room temperature. The reaction mixture was then partitioned between EtOAc and water. The organic layer was separated and washed with brine. The combined organic layers were dried over Na₂SO₄. The residue obtained after filtration and concentration was purified by flash chromatography (0-30% EtOAc in hexane). A Mixture of 44.2X and 44.2 was obtained as a colorless oil (88 mg, 51%). MS ESI (pos.) m/e: 397 (M+H).

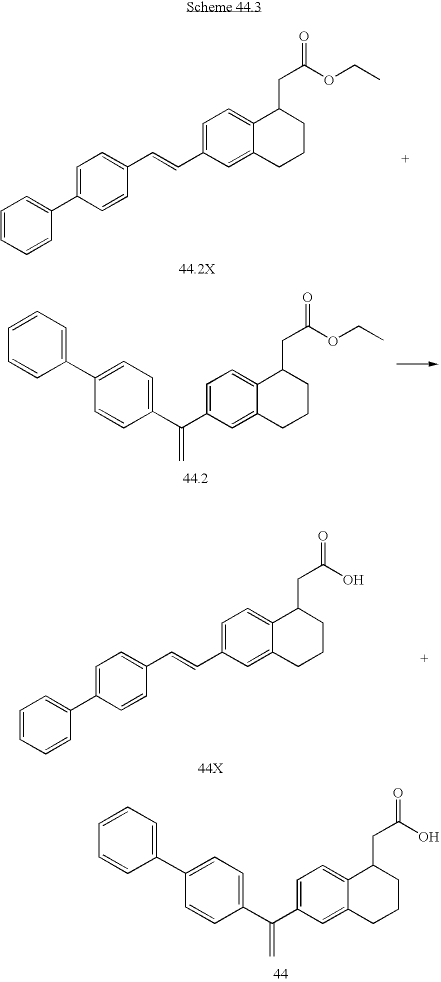

(R/S)-[6-((E)-2-Biphenyl-4-yl-vinyl)-1,2,3,4-tetrahydro-naphthalen-1-yl]-acetic acid (16) and (R/S)-[6-(1-Biphenyl-4-yl-vinyl)-1,2,3,4-tetrahydro-naphthalen-1-yl]-acetic acid (17). A mixture of (44.2) and (44X) (88 mg, 0.22 mmol) in THF-EtOH-H₂O (1/1/1, 6 mL) was treated with LiOH (30 mg). The mixture was stirred at room temperature for 6 hours. 1 N HCl was added to bring the pH of the mixture to about 2-3. The mixture was extracted with EtOAc (2×20 mL). The organic solution was washed with water and brine and was then dried over Na₂SO₄. The residue obtained after filtration and concentration was purified by preparatory HPLC (5-95% acetonitrile-water). Both 44X (51 mg) and 44 (4.7 mg) were obtained as a white solid; combined chemical yield 69%.

(R/S)-[6-((E)-2-Biphenyl-4-yl-vinyl)-1,2,3,4-tetrahydro-naphthalen-1-yl]-acetic acid (44X). MS ESI (neg.) m/e: 367 (M−H). ¹H NMR (500 MHz) (DMSO-d₆) δ 7.68-7.71 (m, 6H); 7.21-7.49 (m, 8H); 3.38-3.42 (m, 1H); 2.70-2.79 (m, 3H); 2.67 (dd, 1H, J=8.0 7.5 Hz); 2.42 (dd, 1H, J=7.5, 5.5 Hz); 1.60-1.80 (m, 3H).

(R/S)-[6-(1-Biphenyl-4-yl-vinyl)-1,2,3,4-tetrahydro-naphthalen-1-yl]-acetic acid (44). MS ESI (neg.) m/e: 367 (M−H). ¹H NMR (500 MHz) CDCl₃) δ 7.10-7.66 (m, 12H); 5.47 (s, 1H); 5.50 (s, 1H); 3.42 (m, 1H); 2.78-2.86 (m, 3H); 2.65 (dd, 1H, J=8.0, 5.0 Hz); 2.01 (m, 1H); 1.72-1.84 (m, 3H).

### Examples 45 and 45X

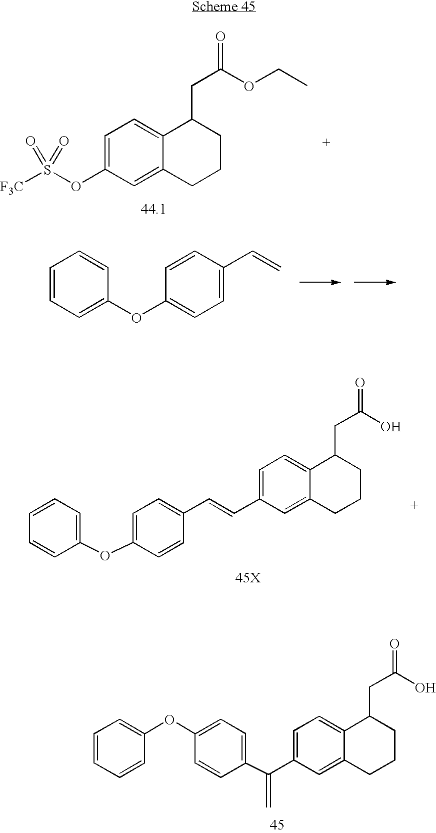

Compounds 45X and 45 were prepared in a manner similar to that of 44X and 44. (R/S)-{6-[(E)-2-(4-Phenoxy-phenyl)-vinyl]-1,2,3,4-tetrahydro-naphthalen-1-yl}-acetic acid (45X). MS ESI (neg.) m/e: 383 (M−H). ¹H NMR (500 MHz) CDCl₃) δ 6.98-7.50 (m, 14H); 3.39-3.42 (m, 1H); 2.78-2.88 (m, 3H); 2.63 (dd, 1H, J=7.5, 5.0 Hz); 1.99-2.04 (m, 1H); 1.80-1.89 (m, 3H).

(R/S)-{6-[1-(4-Phenoxy-phenyl)-vinyl]-1,2,3,4-tetrahydro-naphthalen-1-yl}-acetic acid (45). MS ESI (neg.) m/e: 383 (M−H). ¹H NMR (500 MHz) (DMSO-d₆) δ 6.98-7.40 (m, 12H); 5.41 (m, 2H); 3.41 (m, 1H); 2.76-2.85 (m, 3H); 2.64 (dd, 1H, J=8.0, 5.0 Hz); 1.98-2.01 (m, 1H); 1.75-1.89 (m, 3H).

### Examples 46-47

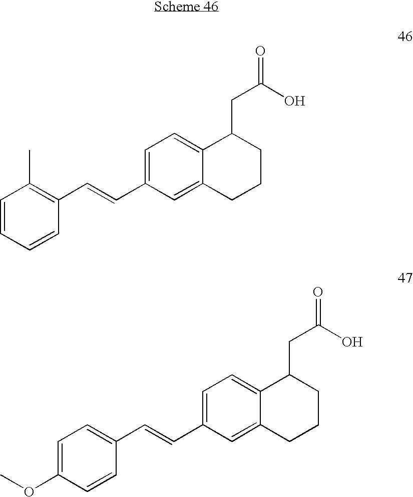

Examples 46 and 47 were prepared using the same procedure used to prepare 44X from 44.1 using the appropriate olefin.

[6-((E)-2-o-Tolyl-vinyl)-1,2,3,4-tetrahydro-naphthalen-1-yl]-acetic acid (46). MS ESI (neg.) m/e: 305 (M−H). ¹H NMR (500 MHz) CDCl₃) δ 7.61 (d, 1H, J=7.4 Hz); 7.20-7.36 (m, 7H); 6.97 (d, 1H, J=16.1 Hz); 3.41 (m, 1H); 2.81-2.87 (m, 3H); 2.64 (dd, 1H, J=9.9; 15.5 Hz); 2.46 (s, 3H); 2.02 (m, 1H); 1.80-1.90 (m, 3H).

{6-[(E)-2-(4-Methoxy-phenyl)-vinyl]-1,2,3,4-tetrahydro-naphthalen-1-yl}-acetic acid (47). MS ESI (neg.) m/e: 321 (M−H). ¹H NMR (500 MHz) CDCl₃) δ 7.46 (d, 2H, J=11.5 Hz); 6.91-7.33 (m, 7H); 3.85 (s, 3H); 3.36 (m, 1H); 2.80-2.84 (m, 3H); 2.62 (dd, 1H, J=9.9, 15.6 Hz); 1.99-2.01 (m, 1H); 1.78-1.90 (m, 3H).

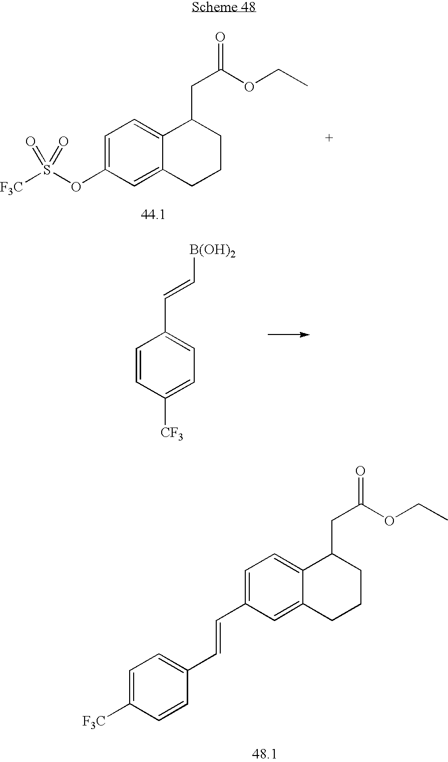

{6-[(E)-2-(4-Trifluoromethyl-phenyl)-vinyl]-1,2,3,4-tetrahydro-naphthalen-1-yl}-acetic acid ethyl ester (48.1). (E)-2-(4-(trifluoromethyl)phenyl)-vinylboronic acid (98 mg, 0.45 mmol) (Aldrich, Milwaukee, Wis.) and CsF (76 mg, 0.50 mmol) were added to a solution of ethyl 2-(6-(trifluoromethylsulfonyloxy)-1,2,3,4-tetrahydronaphthalen-1-yl)acetate 44.1 (110 mg, 0.30 mmol) in dry DME (2 mL) in a 2 dram vial. The mixture was then purged with nitrogen and Pd(PPh₃)₄ (40 mg, 0.035 mmol) was added. The vial was tightly sealed, and the reaction was stirred at 85° C. overnight. After the mixture was cooled to room temperature, the mixture was directly loaded onto a silica gel column for chromatography purification. Product 48.1 was obtained as a colorless oil (122 mg); chemical yield 95%. MS ESI (pos.) m/e: 389 (M+H).

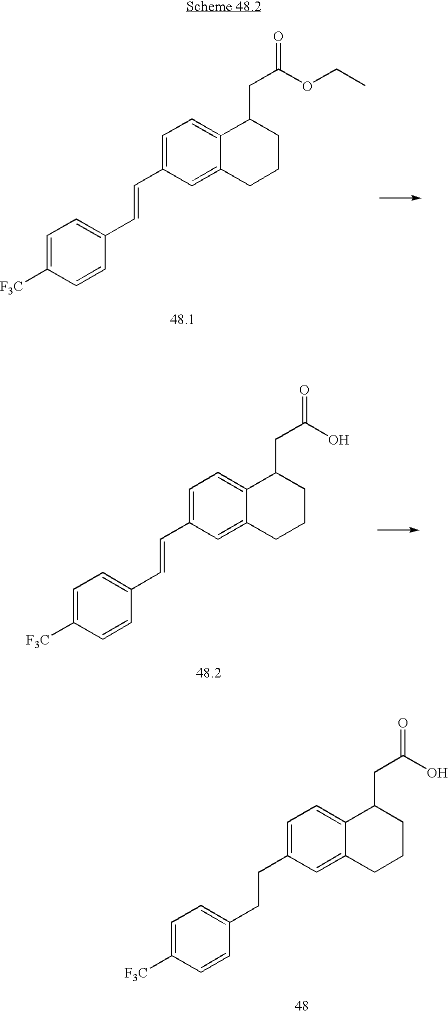

48.2 was prepared from 48.1 using the same procedure used to prepare 44 from 44.2.

{6-[(E)-2-(4-Trifluoromethyl-phenyl)-vinyl]-1,2,3,4-tetrahydro-naphthalen-1-yl}-acetic acid (48.2). MS ESI (neg.) m/e: 359 M−H). ¹H NMR (400 MHz) (CDCl₃) δ 12.20 (s, 1H); 6.90-8.30 (m, 9H); 3.20 (m, 1H); 2.38-2.76 (m, 4H); 1.67-1.84 (m, 4H).

{6-[2-(4-Trifluoromethyl-phenyl)-ethyl]-1,2,3,4-terahydro-naphthalen-1-yl}-acetic acid (48). 48.2 (40 mg) was dissolved in EtOAc (20 mL), and the solution was purged with nitrogen. Under a nitrogen atmosphere, Pd/C (10%) (40 mg) was added. The mixture was purged with H₂, and A H₂ filled balloon was placed on the reaction vessel. The reaction was then stirred overnight at room temperature under balloon pressure hydrogen. The reaction mixture was filtered, and the solvent was removed under reduced pressure. The residue was purified by prep HPLC. 48 was obtained as white solid (40 mg). MS ESI (neg.) m/e: 361 (M−H). ¹H NMR (500 MHz) (CDCl₃) δ 7.57 (d, 2H, J=7.7 Hz); 7.32 (d, 2H, J=7.7 Hz); 7.15 (d, 1H, J=7.7 Hz); 6.70 (d, 1H, J=7.3 Hz); 6.93 (s, 1H); 3.38 (m, 1H); 3.00 (m, 2H); 2.89 (m, 2H); 2.75-2.83 (m, 3H); 2.60-2.64 (m, 1H); 2.00 (m, 1H); 1.80-1.87 (m, 3H).

### Example 49

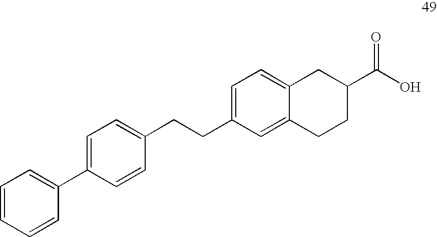

Compound 49 was prepared from 44.1 and the corresponding boronic acid, (E)-2-(4-biphenyl)vinylboronic acid (Aldrich, Milwaukee, Wis.), using the same procedure used to prepare 48 from 44.1.

[6-(2-Biphenyl-4-yl-ethyl)-1,2,3,4-tetrahydro-naphthalen-1-yl]-acetic acid (49). MS ESI (neg.) m/e: 369 (M−H). ¹H NMR (500 MHz) (CDCl₃) δ 6.99-7.63 (m, 12H); 3.38 (m, 1H); 2.91-2.98 (m, 4H); 2.76-2.84 (m, 3H); 2.60-2.64 (dd, 1H, J=10.0, 15.5 Hz); 1.99 (m, 1H); 1.76-1.86 (m, 3H).

### Example 50

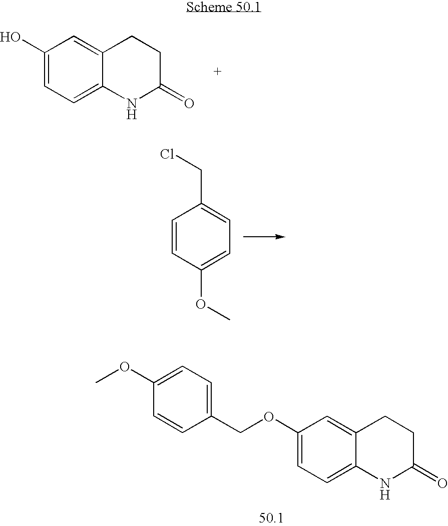

6-(4-Methoxy-benzyloxy)-3,4-dihydro-1H-quinolin-2-one (50.1). Cs₂CO₃ (4.0 g, 12.3 mmol) was added to a solution of 6-hydroxy-3,4-dihydro-1H-quinolin-2-one (1.5 g, 9.2 mmol) in DMF (10 mL). The resulting mixture was stirred at room temperature for 10 minutes. 4-Methoxybenzyl chloride (1.4 g, 9.2 mmol) was then added to the mixture by syringe, and the reaction mixture was stirred at room temperature overnight. The reaction mixture was poured into water and acidified to a pH of 7. The reaction mixture was then extracted with EtOAc (4×20 mL). The combined extracts were washed with water followed by brine. The product was recrystallized from hot EtOAc to yield 50.1 (1.6 g, 60%). ¹H NMR (400 MHz) (DMSO-d₆) δ 7.28 (m, 1H); 6.97 (m, 2H); 6.86 (m, 2H); 6.77 (m, 2H); 5.00 (s, 2H); 3.75 (s, 3H); 2.81 (m, 2H); 2.39 (t, 2H, J=7 Hz). MS ESI (pos.) m/e: 284.0 (M+H).

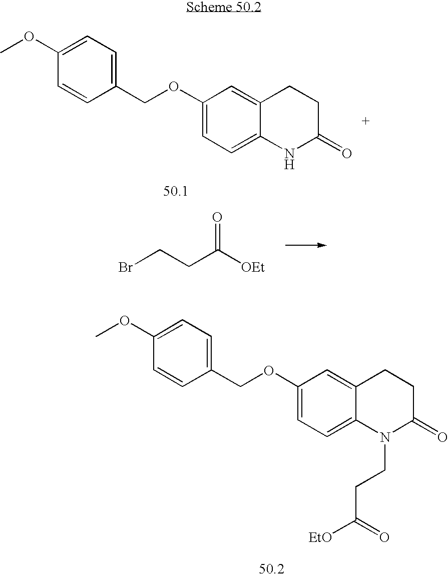

3-[6-(4-Methoxy-benzyloxy)-2-oxo-3,4-dihydro-2H-quinolin-1-yl]-propionic acid ethyl ester (50.2). A pear-shaped flask was charged with 50.1 (0.5 g, 2.1 mmol) in THF (10 mL). NaH (170 mg, 4.2 mmol) was added to the solution in one portion, and the resulting mixture was stirred for 2 minutes. 3-Bromo-propionic acid ethyl ester (0.25 mL, 2.1 mmol) was then added to the reaction mixture by syringe. The resulting mixture was stirred at room temperature overnight. The reaction mixture was concentrated by rotary evaporation, and the resulting residue was partitioned between EtOAc and water. The mixture was extracted with EtOAc (2×20 mL), and the combined extracts were washed with brine. The combined extracts were concentrated, and then purified by radial chromatography (50% EtOAc in hexane) to yield 50.2 (178 mg, 22%). MS ESI (pos.) m/e: 384.1 (M+H).

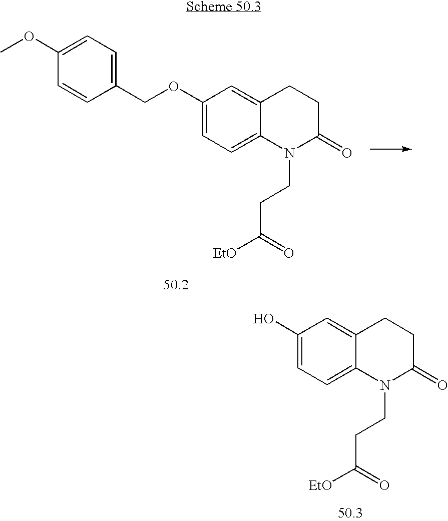

3-(6-Hydroxy-2-oxo-3,4-dihydro-2H-quinolin-1-yl)-propionic acid ethyl ester (50.3). A pear-shaped flask was charged with 50.2 (178 mg, 0.46 mmol), 10% Pd/C (20 mg), and EtOH (5 mL). A hydrogen filled balloon was attached to the reaction vessel, and the vessel was evacuated and backfilled with hydrogen three times. The reaction was stirred vigorously under a hydrogen atmosphere overnight. The reaction mixture was then filtered through a plug of Celite® to remove the Pd/C, and the resulting solution was purified by radial chromatography (10% MeOH in DCM) to yield 50.3 (111 mg, 92%). MS ESI (pos.) m/e: 264.1 (M+H).

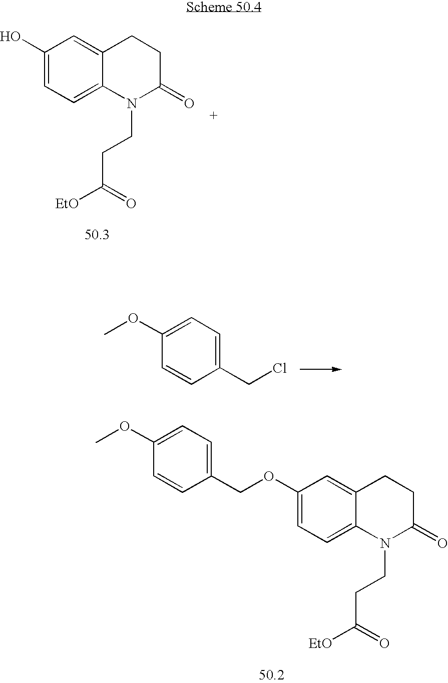

3-[6-(4-Methoxy-benzyloxy)-2-oxo-3,4-dihydro-2H-quinolin-1-yl]-propionic acid ethyl ester (50.2). A pear-shaped flask was charged with 50.3 (27 mg, 0.103 mmol), Cs₂CO₃ (67 mg, 0.205 mmol), and acetone (5 mL). The resulting mixture was stirred at room temperature for 5 minutes. 4-Methoxybenzyl chloride (14 μL, 0.103 mmol) was added in one portion, and the resulting mixture was stirred at room temperature overnight. The reaction mixture was concentrated, and the resulting residue was purified by radial chromatography (50% EtOAc in hexane) to yield 50.2 (24 mg, 61%). MS ESI (pos.) m/e: 384.1 (M+H).

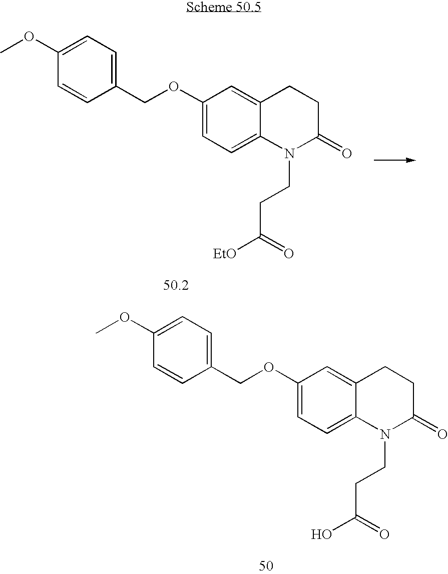

3-[6-(4-Methoxy-benzyloxy)-2-oxo-3,4-dihydro-2H-quinolin-1-yl]-propionic acid (50). To a solution of 50.2 (24 mg, 0.06 mmol) in EtOH (3 mL), was added 2 N NaOH (1 mL, 2.0 mmol). The reaction mixture was stirred at room temperature overnight. The reaction mixture was then partitioned between EtOAc and 1 N HCl. The mixture was extracted with EtOAc (2×5 mL). The combined extracts were concentrated, and the resulting residue was purified by radial chromatography (4% MeOH in hexane) followed by reverse phase HPLC (30%-70% acetonitrile in H₂O) to yield 50 (14 mg, 65%). ¹H NMR (400 MHz) (Acetone-d₆) δ 7.38 (m, 1H); 7.09 (m, 1H); 6.94 (m, 2H); 6.90 (m, 2H); 6.71 (m, 1H); 5.01 (s, 2H); 4.15 (m, 2H); 3.80 (s, 31H); 2.83 (m, 2H); 2.65 (m, 2H); 2.52 (m, 2H). MS ESI (pos.) m/e: 356.0 (M+H).

### Examples 51-53

Compounds 51-53 were prepared from 5-benzyloxy-1H-indole using a method similar to that described for Example 50.

### Example 54

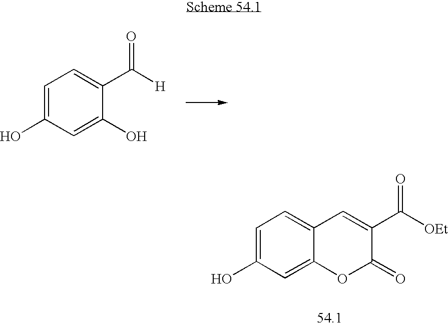

Ethyl 7-hydroxy-2-oxo-2H-chromene-3-carboxylate (54.1). 2,6-Dihydoxybenzaldehyde (2.76 g, 20 mmol), diethylmalonate (9 mL, 60 mmol), and piperidine (2 mL, 20 mmol) were mixed and stirred at room temperature for 3 hours. The product solidified as reaction went to completion. The reaction mixture was washed with EtOAc and dried to give a product which was a co-crystal of 54.1 and piperidine (5 g, 85% yield). MS ESI (pos.) m/e: 235.1 (M+H). ¹H NMR (400 MHz) CDCl₃) δ 8.29 (s, 1H); 7.28 (s, 1H); 7.21 (d, 1H); 6.60 (dd, 1H); 6.47 (d, 1H); 4.30 (q, 2H); 3.10 (m, 4H); 1.79 (m, 4H); 1.67 (m, 2H); 1.39 (t, 3H).

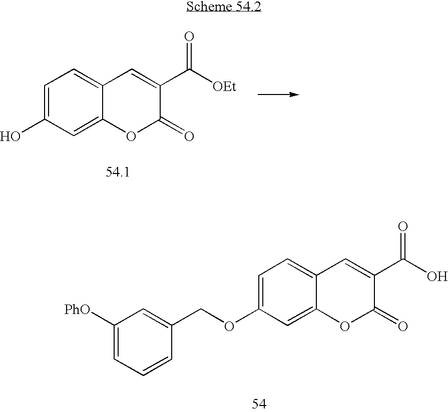

7-(3-Phenoxybenzyloxy)-2-oxo-2H-chromene-3-carboxylic acid (54). 54.1 (2.34 g, 7.3 mmol), 3-phenoxybenzyl chloride (2.18 g, 10 mmol) and K₂CO₃ (2.76 g, 20 mmol) were mixed in DMF and stirred at 50° C. for 14 hours. Additional 3-phenoxybenzyl chloride (1.1 g, 5 mmol) was added, and the reaction was continued for 2 more hours. After cooling, the mixture was treated with water (100 mL) and EtOAc (200 mL). The organic layer was separated, was washed twice with brine, was dried over MgSO₄ and concentrated under vacuum. The crude product was purified by flash column chromatography providing the ethyl ester of 54 (2 g, 67% yield). MS ESI (pos.) m/e: 417.1 (M+H). ¹H NMR (400 MHz) CDCl₃) δ 8.52 (s, 1H); 7.52 (d, 1H); 7.37 (m, 3H); 7.20-6.90 (m, 7H); 6.87 (d, 1H); 5.15 (s, 2H); 4.42 (q, 2H); 1.43 (t, 3H).

A solution of the ethyl ester of 54 (46 mg, 0.11 mmol) and LiOH monohydrate (25 mg, 0.625 mmol) in 2.5 mL THF/MeOH/water (2:2:1), was stirred at room temperature for 4 hours. The organic solvent was removed by blowing air over the mixture. The resultant aqueous solution was acidified by adding 3 N HCl. The resulting mixture was then extracted with DCM. The organic layer was separated, was washed twice with brine, was dried with MgSO₄, and concentrated under vacuum. The crude product was purified by flash column chromatography providing 54 (35 mg). MS ESI (pos.) m/e: 389.1 (M+H). ¹H NMR (400 MHz) (DMSO-d₆) δ 8.71 (s, 1H); 7.85 (d, 1H); 7.50-6.90 (m, 1H); 5.25 (s, 2H).

### Example 55

Compound 55 was prepared from compound 1.5 and compound F according to the methods described in Example 1.

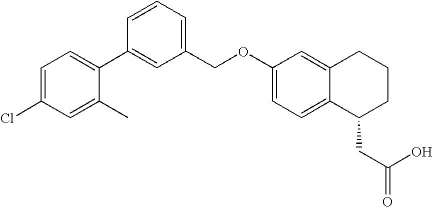

(R)-6(4′-Chloro-2′-methyl-biphenyl-3-ylmethoxy)-1,2,3,4-tetrahydro-naphthalen-1-yl]-acetic acid (55). LC-MS ESI (neg.) m/e: 419.1 (M−H). ¹H NMR (500 MHz) (CDCl₃) δ 7.45 (m, 2H); 7.36 (s, 1H); 7.23-7.29 (m, 3H); 7.18 (d, 1H); 7.13 (d, 1H); 6.82 (dd, 1H); 6.73 (d, 1H); 5.10 (s, 2H); 3.34 (m, 1H); 2.76 (m, 3H); 2.59 (dd, 1H); 2.25 (s, 3H); 1.97 (m, 1H); 1.80 (m, 3H).

### Example 56

Example 56 was prepared from compound 1.5 and commercially available 5-(chloromethyl)-4-methyl-2-(4-(trifluoromethyl)phenyl)thiazole (available from Key Organics/Bionet) according to the methods described in Example 1.

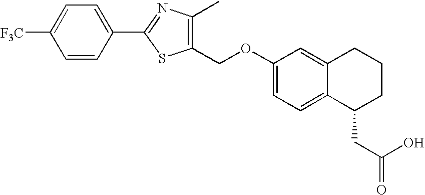

(R)-6-(4-Trifluoromethyl-2-p-tolyl-thiazol-5-ylmethoxy)-1,2,3,4-tetrahydro-naphthalen-1-yl]-acetic acid (56). LC-MS ESI (neg.) m/e: 460.1 (M−H). ¹H NMR (500 MHz) (CDCl₃) δ 8.02 (d, 2H); 7.74 (d, 2H); 7.14 (d, 1H); 6.79 (dd, 1H); 6.71 (d, 1H); 5.19 (s, 2H); 3.33 (m, 1H); 2.76 (m, 3H); 2.60 (dd, 1H); 2.56 (s, 3H); 1.95 (m, 1H); 1.80 (m, 3H).

### Example 57

Example 57 was prepared from compound 1.4 and compound G according to the methods described in Example 1.

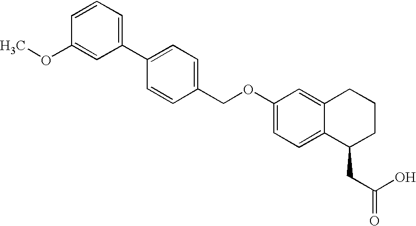

(S)-[6-(3′-Methoxy-biphenyl-4-ylmethoxy)-1,2,3,4-tetrahydro-naphthalen-1-yl]-acetic acid (57). LC-MS ESI (neg.) m/e: 401.2 (M−H). ¹H NMR (500 MHz) (CDCl₃) δ 7.65 (d, 2H); 7.53 (d, 2H); 7.40 (t, 1H); 7.22 (d, 1H); 7.17 (m, 2H); 6.95 (dd, 1H); 6.85 (dd, 1H); 6.77 (dd, 1H); 5.11 (s, 2H); 3.91 (s, 3H); 3.37 (m, 1H); 2.80 (m, 3H); 2.62 (dd, 1H); 1.87 (m, 1H); 1.82 (m, 3H).

### Example 58

Example 58 was prepared from compound 1.5 and compound G according to the methods described in Example 1.

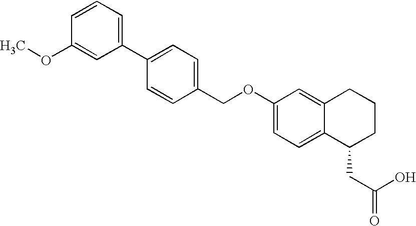

(R)-[6-(3′-Methoxy-biphenyl-4-ylmethoxy)-1,2,3,4-tetrahydro-naphthalen-1-yl]-acetic acid (58). LC-MS ESI (neg.) m/e: 401.2 (M−H). ¹H NMR (500 MHz) (CDCl₃) δ 7.65 (d, 2H); 7.53 (d, 2H); 7.40 (t, 1H); 7.22 (d, 1H); 7.17 (m, 2H); 6.95 (dd, 1H); 6.85 (dd, 1H); 6.77 (dd, 1H); 5.11 (s, 2H); 3.91 (s, 3H); 3.37 (m, 1H); 2.80 (m, 3H); 2.62 (dd, 1H); 1.87 (m, 1H); 1.82 (m, 3H).

### Example 59

Example 59 was prepared from compound 1.4 and compound H according to the methods described in Example 1

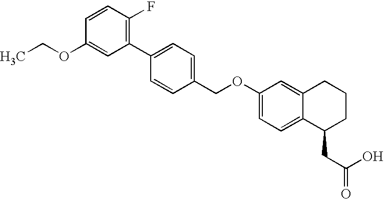

(S)-[6-(5′-Ethoxy-2′-fluoro-biphenyl-4-ylmethoxy)-1,2,3,4-tetrahydro-naphthalen-1-yl]-acetic acid (59): LC-MS ESI (neg.) m/e: 433.2 (M−H). ¹H NMR (500 MHz) (CDCl₃) δ 7.60 (d, 2H); 7.53 (d, 2H); 7.15 (d, 2H); 7.09 (t, 1H); 6.98 (dd, 1H); 6.85 (m, 1H); 6.76 (d, 1H); 5.10 (s, 2H); 4.07 (q, 2H); 3.36 (m, 1H); 2.79 (m, 3H); 2.60 (dd, 1H); 1.98 (m, 1H); 1.79 (m, 3H); 1.45 (t, 3H).

### Example 60

Example 60 was prepared from compound 1.5 and compound H according to the methods described in Example 1.

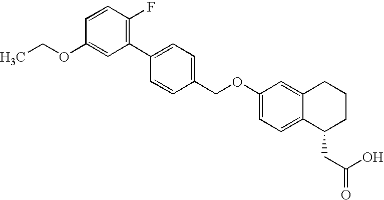

(S)-[6-(5′-Ethoxy-2′-fluoro-biphenyl-4-ylmethoxy)-1,2,3,4-tetrahydro-naphthalen-1-yl]-acetic acid (60). LC-MS ESI (neg.) m/e: 433.2 (M−H). ¹H NMR (500 MHz) (CDCl₃) δ 7.60 (d, 2H); 7.53 (d, 2H); 7.15 (d, 2H); 7.09 (t, 1H); 6.98 (dd, 1H); 6.85 (m, 1H); 6.76 (d, 1H); 5.10 (s, 2H); 4.07 (q, 2H); 3.36 (m, 1H); 2.79 (m, 3H); 2.60 (dd, 1H); 1.98 (m, 1H); 1.79 (m, 3H); 1.45 (t, 3H).

### Examples 61-63

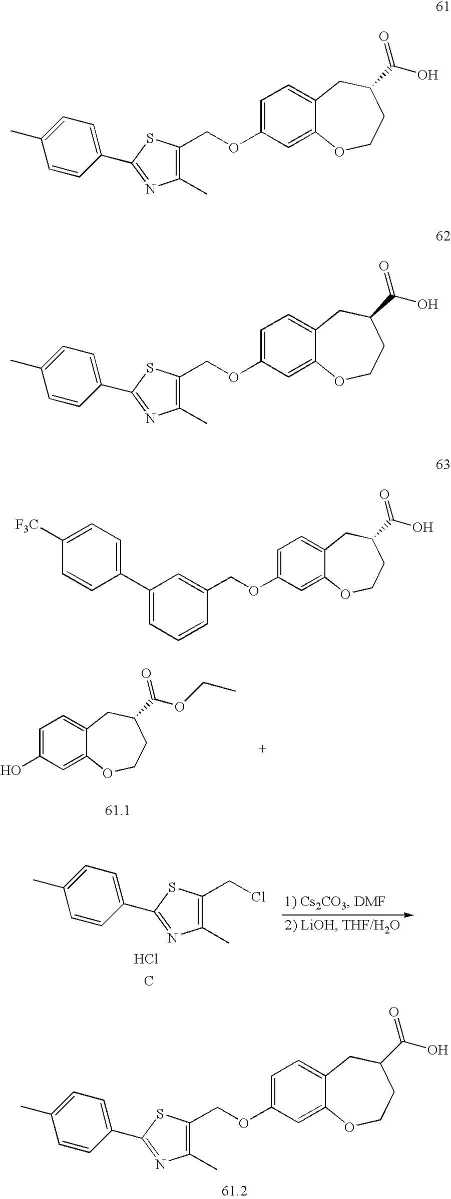

8-((4-Methyl-2-p-tolylthiazol-5-yl)methoxy)-2,3,4,5-tetrahydrobenzo[b]oxepine-4-carboxylic acid (61.2). Cesium carbonate (341 mg, 1.04 mmol) was added into a mixture of ethyl 8-hydroxy-2,3,4,5-tetrahydrobenzo[b]oxepine-4-carboxylate (61.1) (100 mg, 0.42 mmol) (prepared according to WO 2004/106276) and 5-(chloromethyl)-4-methyl-2-p-tolylthiazole hydrochloride (C, 121 mg, 0.44 mmol) in DMF (5 mL). The mixture was stirred at room temperature for 14 hours. Purification by flash chromatography provided the corresponding ester (169 mg, 0.39 mmol). The ester was dissolved in THF (2 mL), LiOH in water (1 mL, 2N solution) was added, and the reaction was stirred at 80° C. for 3 hours. The resulting mixture was filtered and purified by reverse phase HPLC to give 61.2 (105 mg) after lyophilization. MS ESI (pos.) m/e 410.2 (M+H). ¹H NMR (500 MHz, CDCl₃) δ ppm 7.82 (2H, d, J=8.2 Hz), 7.25 (2H, d, J=7.9 Hz), 7.12 (2H, d, J=8.2 Hz), 6.67 (2H, td, J=8.2, 2.4 Hz), 5.15 (2H, s), 4.31-4.35 (1H, m), 3.03-3.14 (2H, m), 2.50 (3H, s), 2.41 (3H, s), 2.24-2.29 (2H, m).

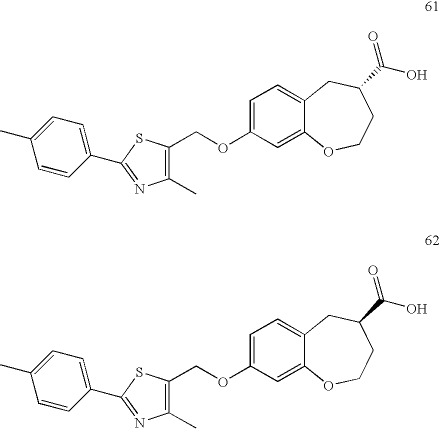

The racemic compound 61.2 was separated into two enantiomers 61 (32 mg, first peak) and 62 (31 mg, second peak) using a chiral preparative AD-H column (20% IPA/80% hexanes).

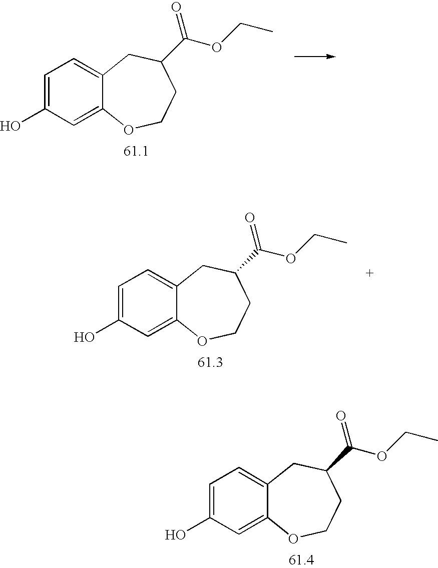

The racemic compound 61.1 was separated into two enantiomers 61.3 (first peak) and 61.4 (second peak) using a chiral preparative AD-H column (20% IPA/80% hexanes).

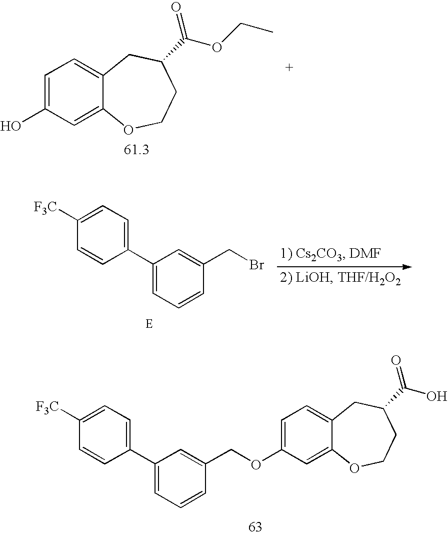

(S)-8-(3-(4-Trifluoromethylphenyl)-phenyl)methoxy)-2,3,4,5-tetrahydrobenzo[b]oxepine-4-carboxylic acid (63). Cesium carbonate (139 mg, 0.43 mmol) was added to a mixture of 61.3 (80 mg, 0.34 mmol) and E (112 mg, 0.36 mmol) in DMF (2 mL). The mixture was stirred at room temperature for 14 hours. Purification by flash chromatography provided the corresponding ester. The ester was dissolved in THF (1 mL), and LiOH (21 mg) and H₂O₂ were added in water (1 mL, 33%). The mixture was stirred at 50° C. for 3 hours. The resulting mixture was filtered and purified by reverse phase HPLC to give 63 (56 mg) after lyophilization. MS ESI (pos.) m/e 443.1 (M+H). ¹H NMR (500 MHz, CDCl₃) δ ppm 7.68 (1H, s), 7.57-7.60 (1H, m), 7.47-7.53 (2H, m), 7.29 (1H, s), 7.11 (1H, d, J=8.3 Hz), 6.67-6.71 (2H, m), 5.12 (2H, s), 4.33 (1H, td, J=8.7, 3.9 Hz), 3.89-3.87 (1H, m), 3.08-3.12 (1H, m), 3.05-3.02 (1H, m), 2.28-2.24 (1H, m).

### Example 64

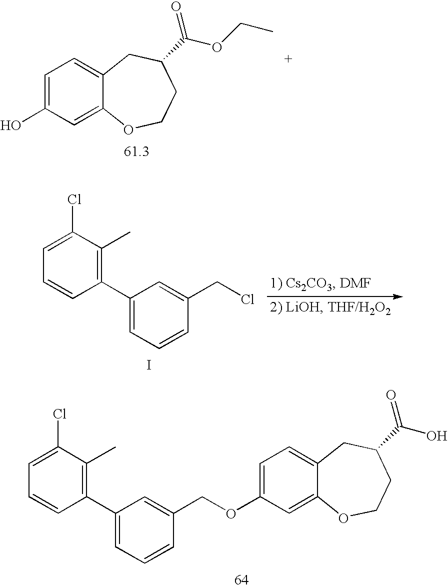

(S)-8-(3-(3-Chloro-2-methylphenyl)-phenyl)methoxy)-2,3,4,5-tetrahydrobenzo[b]oxepine-4-carboxylic acid (64). Cesium carbonate (139 mg, 0.43 mmol) was added into a mixture of 61.3 (80 mg, 0.34 mmol) and 1 (85 mg, 0.36 mmol) in DMF (2 mL). The mixture was stirred at room temperature for 14 hours. Purification by flash chromatography gave the corresponding ester. The ester was dissolved in THF (1 mL), and LiOH (21 mg) and H₂O₂ in water (1 mL, 33%) were added. The mixture was stirred at 50° C. for 3 hours. The reaction was filtered and purified by reverse phase HPLC to give 64 (76 mg) after lyophilization. MS ESI (pos.) m/e 423.1 (M+H). ¹H NMR (500 MHz, CDCl₃) δ ppm 7.43-7.48 (2H, m), 7.39 (1H, d, J=7.6 Hz), 7.36 (1H, s), 7.25-7.27 (1H, m), 7.15-7.21 (2H, m), 7.10 (1H, d, J=8.1 Hz), 6.65-6.69 (2H, m), 5.09 (2H, s), 4.30-4.35 (1H, m), 3.84-3.89 (1H, m), 3.08-3.11 (1H, m), 3.02-3.04 (1H, m), 2.29 (3H, s), 2.23-2.26 (1H, m).

### Example 65

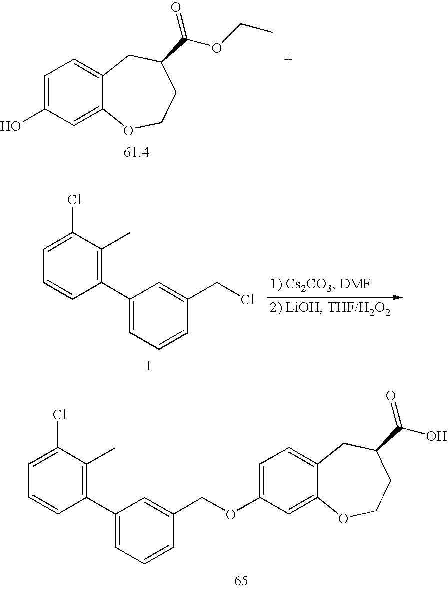

(R)-8-(3-(3-Chloro-2-methylphenyl)-phenyl)methoxy)-2,3,4,5-tetrahydrobenzo[b]oxepine-4-carboxylic acid (64). Cesium carbonate (139 mg, 0.43 mmol) was added into a mixture of 61.4 (80 mg, 0.34 mmol) and 1 (85 mg, 0.36 mmol) in DMF (2 mL). The mixture was stirred at room temperature for 14 hours. Purification by flash chromatography gave the corresponding ester. The ester was dissolved in THF (1 mL), and LiOH (21 mg) and H₂O₂ in water (1 mL, 33%) were added. The mixture was stirred at 50° C. for 3 hours. The reaction was filtered and purified by reverse phase HPLC to give 65 (84 mg) after lyophilization. MS ESI (pos.) m/e 423.1 (M+H). 1H NMR (500 MHz, CDCl₃) δ ppm 7.43-7.48 (2H, m), 7.39 (1H, d, J=7.6 Hz), 7.36 (1H, s), 7.25-7.27 (1H, m), 7.15-7.21 (2H, m), 7.10 (1H, d, J=8.1 Hz), 6.65-6.69 (2H, m), 5.09(2H, s), 4.30-4.35 (1H, m), 3.84-3.89 (1H, m), 3.08-3.11 (1H, m), 3.02-3.04 (1H, m), 2.29 (3H, s), 2.23-2.26 (1H, m).

### Example 66

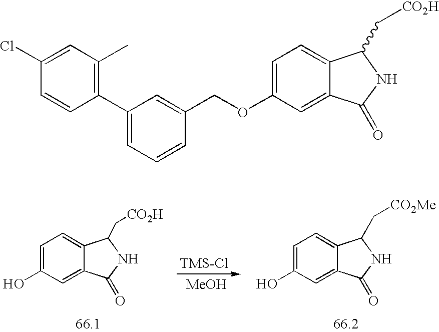

(R/S)-methyl 2-(5-hydroxy-3-oxoisoindolin-1-yl)acetate (66.2). Chlorotrimethylsilane (270 mg, 2.5 mmol) was added to a suspension of 66.1 (515 mg, 2.5 mmol, commercially available from Matrix Scientific) in MeOH (10 mL). The mixture was stirred at room temperature for 14 hours. After concentration under reduced pressure, the crude methyl ester was used without further purification in the next reaction.

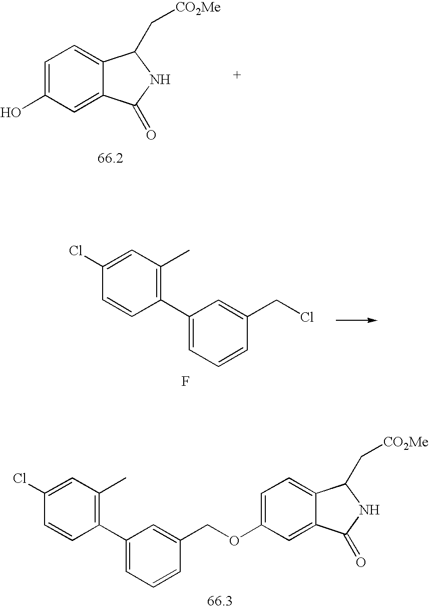

(R/S)-Methyl (5-(((4′-chloro-2′-methyl-1,1′-biphenyl-3-yl)methyl)oxy)-3-oxo-2,3-dihydro-1H-isoindol-1-yl)acetate (66.3). Compound 66.2 (224 mg, 1.01 mmol) and compound F (255 mg, 1.01 mmol) were converted to the title compound according to the method given in Example 1.

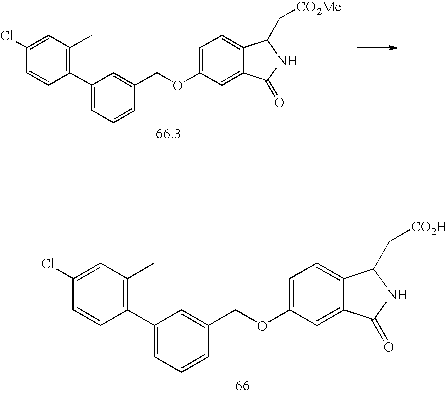

(R/S)-(5-(((4′-chloro-2′-methyl-1,1′-biphenyl-3-yl)methyl)oxy)-3-oxo-2,3-dihydro-1H-isoindol-1-yl)acetic acid (66). Compound 66.3 (224 mg, 1.01 mmol) was hydrolyzed according to the method given in Example 1. ¹H NMR (400 MHz) (DMSO-d₆) δ 8.59 (s, 1H), 7.52-7.48 (m, 4H), 7.41 (d, 2H), 7.32 (m, 2H), 7.26-7.22 (m, 4H), 5.43 (s, 2H), 4.79 (t, 1H), 2.75 (dd, 1H), 2.20 (s, 3H).

### Example 67

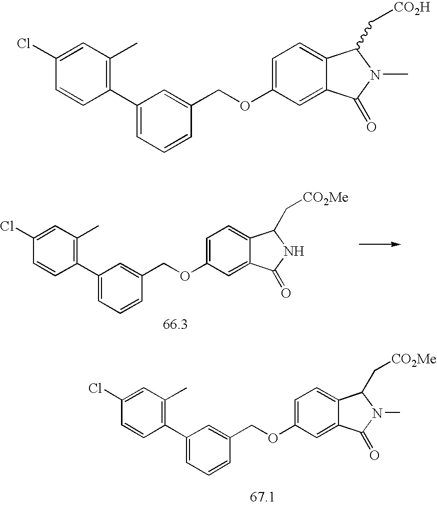

(R/S)-Methyl (5-(((4′-chloro-2′-methyl-1,1′-biphenyl-3-yl)methyl)oxy)-2-methyl-3-oxo-2,3-dihydro-1H-isoindol-1-yl)acetate (67.1). Compound 66.3 (44 mg, 0.1 mmol) and MeI (28 mg, 0.2 mmol) were dissolved in DMF (1 mL) and treated with Cs₂CO₃ (65 mg, 0.2 mmol). After stirring at room temperature for 14 hours, the reaction mixture was worked up and the residue triturated with EtOAc:hexanes (1:3) to obtain the N-methylated compound 67.1.

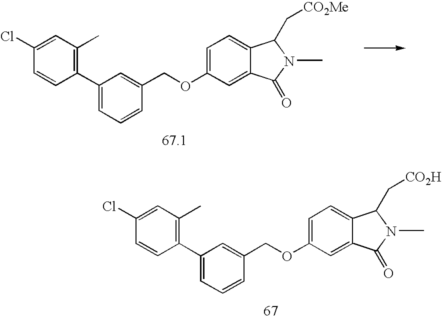

(R/S)-(5-(((4′-chloro-2′-methyl-1,1′-biphenyl-3-yl)methyl)oxy)-2-methyl-3-oxo-2,3-dihydro-1H-isoindol-1-yl)acetic acid (67). Compound 67.1 was hydrolyzed by the method given in Example 1 to yield the title compound 67 (23 mg). LC-MS ESI (pos.) m/e: 436.1 (M+H). ¹H NMR (400 MHz) (DMSO-d₆) δ 7.55 (m, 3H), 7.42 (d, 2H), 7.29 (m, 2H), 7.21 (m, 31H), 5.25 (s, 2H), 4.76 (t, 1H). 3.0 (s, 31H), 2.9 (d, 1H), 2.64 (dd, 1H), 2.21 (s, 3H).

### Example 68

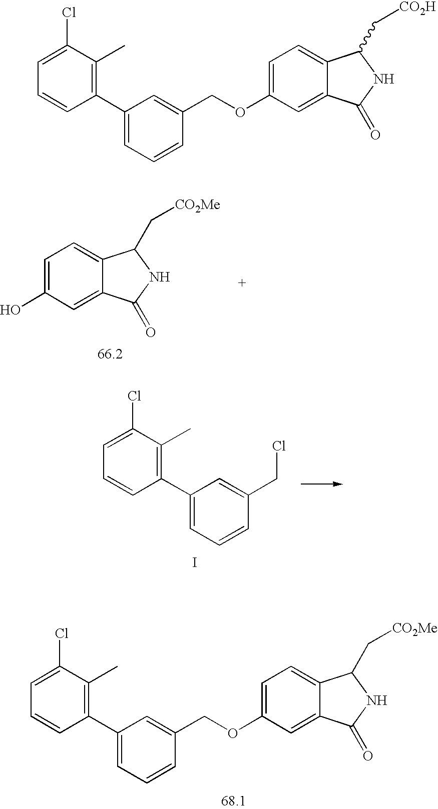

(R/S)-Methyl (5-(((3′-chloro-2′-methyl-1,1′-biphenyl-3-yl)methyl)oxy)-3-oxo-2,3-dihydro-1H-isoindol-1-yl)acetate (68.1). Compound 66.2 was alkylated with Compound I according to the method given in Example 1.

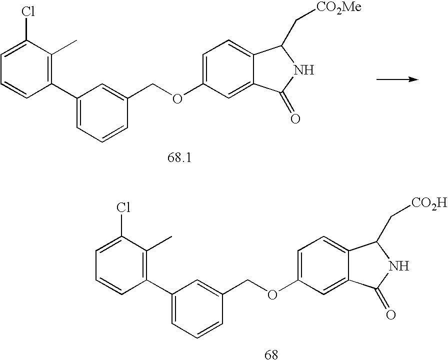

(R/S)-5-(((3′-chloro-2′-methyl-1,1′-biphenyl-3-yl)methyl)oxy)-3-oxo-2,3-dihydro-1H-isoindol-1-yl)acetic acid (68). Compound 68.1 was hydrolyzed according to the method given in Example 1. LC-MS ESI (neg.) m/e: 420.1 (M−H). ¹H NMR (400 MHz) (CDCl₃) δ 8.45 (s, 1H), 7.45 (m, 3H), 7.37 (m, 3H), 7.18 (m, 2H), 5.16 (s, 2H), 4.97 (d, 1H), 3.19 (d, 1H), 2.41 (d, 1H), 2.30 (s, 3H).

### Example 69

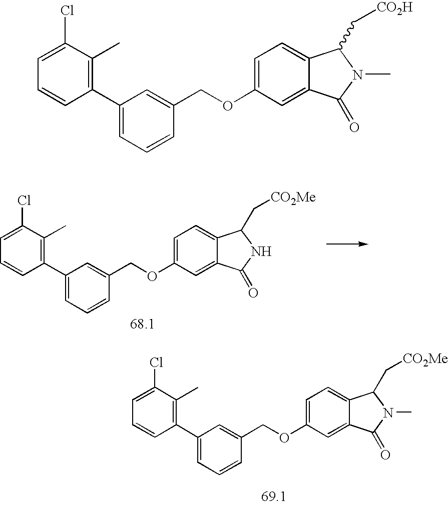

(R/S)-Methyl (5-(((3′-chloro-2′-methyl-1,1′-biphenyl-3-yl)methyl)oxy)-2-methyl-3-oxo-2,3-dihydro-1H-isoindol-1-yl)acetate (69.1). Compound 68.1 was N-methylated according to the method given in Example 67.

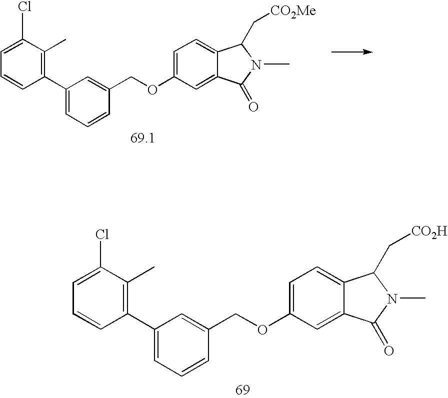

(R/S)-(5-(((3′-chloro-2′-methyl-1,1′-biphenyl-3-yl)methyl)oxy)-2-methyl-3-oxo-2,3-dihydro-1H-isoindol-1-yl)acetic acid (69). Compound 69.1 was hydrolyzed by the method given in Example 1 to provide the title compound 69 (25 mg). LC-MS ESI (neg.) m/e: 434.0 (M−H). ¹H NMR (400 MHz) (CDCl₃) δ 7.48-7.35 (m, 6H), 7.21-7.18 (m, 3H), 5.19 (s, 2H), 4.91 (t, 1H), 3.18 (s, 3H), 2.97 (d, 1H), 2.74 (dd, 1H), 2.30 (s, 3H).

### Example 70

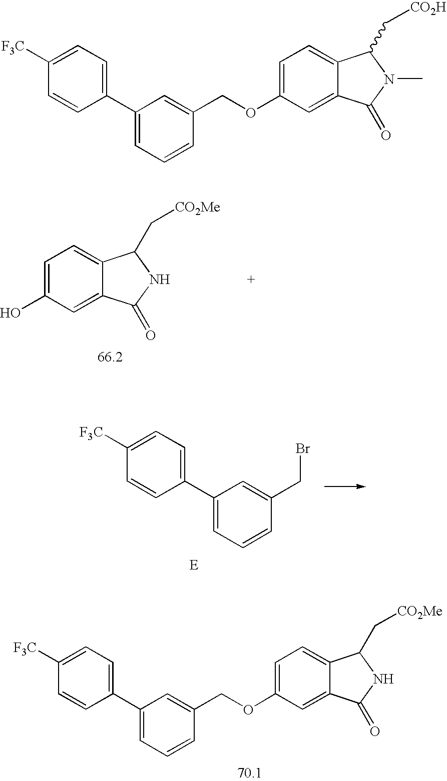

(R/S)-Methyl (3-oxo-5-(((4′-(trifluoromethyl)-1,1′-biphenyl-3-yl)methyl)oxy)-2,3-dihydro-1H-isoindol-1-yl)acetate (70.1). Compound 66.2 was alkylated with Compound E according to the method given in Example 1.

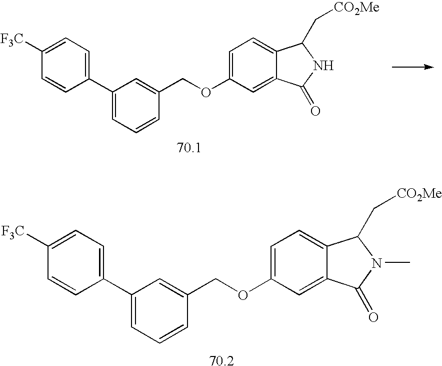

(R/S)-Methyl (2-methyl-3-oxo-5-(((4′-(trifluoromethyl)-1,1′-biphenyl-3-yl)methyl)oxy)-2,3-dihydro-1H-isoindol-1-yl)acetate (70.2). Compound 70.1 was N-methylated according to the method given in Example 67.

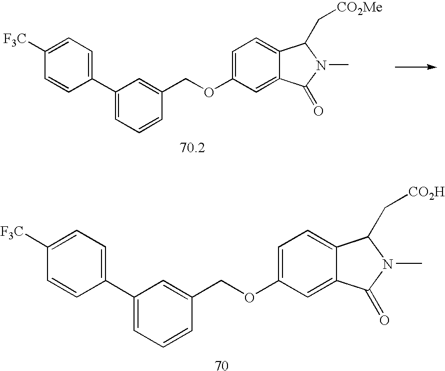

(R/S)-(2-methyl-3-oxo-5-(((4′-(trifluoromethyl)-1,1′-biphenyl-3-yl)methyl)oxy)-2,3-dihydro-1H-isoindol-1-yl)acetic acid (70). Compound 70.2 was hydrolyzed by the method given in Example 1 to yield the title compound 70 (36 mg). LC-MS ESI (pos.) m/e: 456.1 (M+H). ¹H NMR (400 MHz) (DMSO-d₆) δ 7.91 (m, 2H), 7.84 (m, 3H), 7.71 (br s, 1H), 7.52 (m, 3H), 7.27 (m, 2H), 5.28 (s, 2H), 4.76 (t, 1H). 3.0 (s, 3H), 2.92 (dd, 1H), 2.65 (dd, 1H).

### Example 71

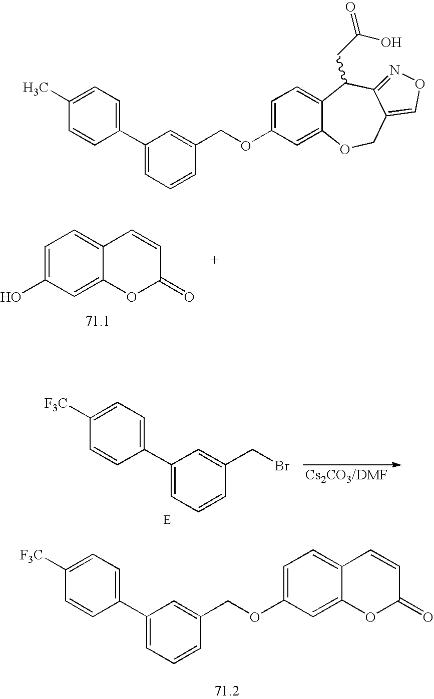

7-(3-(4-(Trifluoromethyl)phenyl)benzyloxy)-2H-chromen-2-one (71.2). 7-Hydroxycoumarin 71.1 (3.24 g, 20 mmol) and the bromide E (6.3 g, 20 mmol) were dissolved in DMF (30 mL). Cs₂CO₃ (14.3 g, 44 mmol) was added portion wise into the solution at room temperature. The mixture was then stirred at 45° C. overnight. After cooling, the mixture was treated with water(100 mL) and acidified to pH˜6 with 3 N HCl (˜30 mL). The solid was collected by filtration, washed with water and dried to give 71.2 (7.5 g, 95% yield). MS ESI (pos.) m/e: 397.1 (M+H). ¹H NMR (400 MHz) (DMSO-d₆) δ 8.01(d, 1H); 7.92(d, 2H), 7.85(m, 3H), 7.73(m, 1H), 7.66(d, 1H), 7.56(m, 2H), 7.13(d, 1H), 7.07(dd, 1H), 5.33(s, 2H).

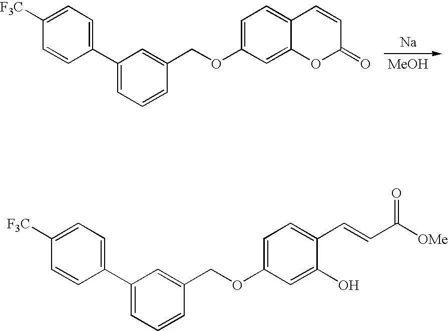

(E)-Methyl 3-(4-(3-(4-(Trifluoromethyl)phenyl)benzyloxy)-2-hydroxyphenyl)acrylate (71.3). Sodium (1 g, 43 mmol) was added in small pieces into dry MeOH (60 mL) at room temperature. Compound 71.2 (4 g, 10 mmol) was then added in small portions. The mixture was stirred at 65° C. for 12 hours. After cooling to 0° C., the mixture was neutralized using 3 N HCl (14.3 mL), and diluted with water (200 mL). The solid was collected by filtration, washed with water and dried to give compound 71.3 (4.1 g, 98% yield). LC-MS ESI (pos.) m/e: 429.1 (M+H). ¹H NMR (400 MHz) (DMSO-d₆) δ 9.40(bs, 1H), 7.92(m, 2H), 7.82(m, 4H), 7.72(m, 1H), 7.55(m, 3H), 6.57(m, 2H), 6.48(d, 1H), 5.20(s, 2H), 3.69(s, 3H).

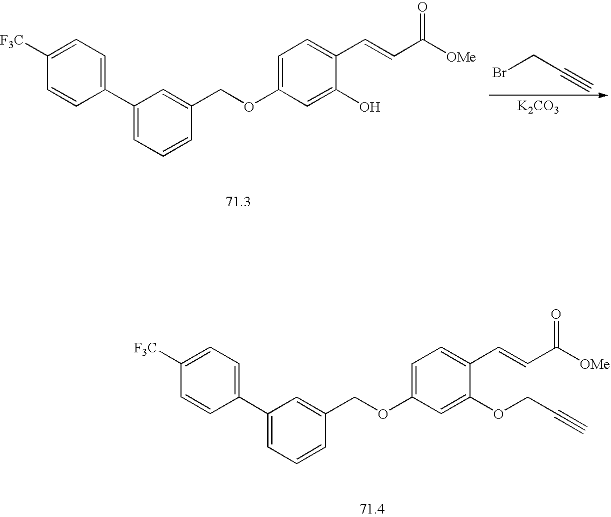

(E)-methyl 3-(4-(3-(4-(Trifluoromethyl)phenyl)benzyloxy)-2-(prop-2-ynyloxy)phenyl)acrylate (71.4). Compound 71.3 (430 mg, 1 mmol) and propargyl bromide (0.11 mL, 1 mmol) were dissolved in DMF (2 mL). K₂CO₃ (152 mg, 1.1 mmol) was added to the solution at room temperature. The mixture was then stirred at 25° C. for 12 hours. The mixture was treated with water (10 mL) and EtOAc (20 mL). The organic layer was separated, washed twice with brine, dried over MgSO₄, and concentrated under vacuum. The crude product was purified with flash column chromatography to give compound 71.4 (440 mg, 95% yield). MS ESI (pos.) m/e: 467.1 (M+H).

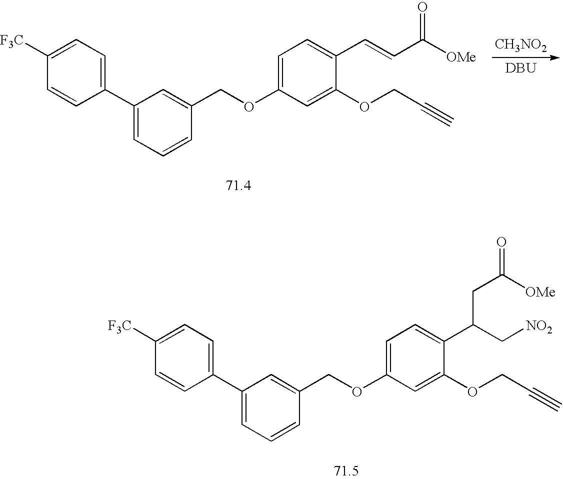

(R/S)-Methyl 3-(4-(3-(4-(Trifluoromethyl)phenyl)benzyloxy)-2-(prop-2-ynyloxy)phenyl)-4-nitrobutanoate (71.5). A mixture of compound 71.4 (80 mg, 0.17 mmol) and DBU (4.3 mg, 0.028 mmol) in nitromethane (0.2 mL) were heated to 160° C. in a microwave reactor for 3 hours. After cooling, the mixture was treated with water (10 mL) and EtOAc (20 mL). The organic layer was separated, washed with brine twice, dried with MgSO₄ and concentrated under vacuum. The crude product was purified with flash column to give compound 71.5 (15 mg, 17% yield). LC-MS ESI (pos.) m/e: 528.1 (M+H). ¹H NMR (400 MHz) (CDCl₃) δ 7.81 (d, 2H); 7.26 (d, 2H); 7.05 (d, 1H); 6.58 (dd, 1H); 6.48 (d, 1H); 6.20 (bs, 1H); 5.20 (s, 1H); 5.16 (s, 1H); 5.14 (s, 2H); 4.64 (d, 1H); 4.50 (d, 1H); 3.91 (dd, 1H); 3.71 (s, 3H); 2.80 (dd, 1H); 2.68 (dd, 1H); 2.52 (s, 31H); 2.41 (s, 3H).

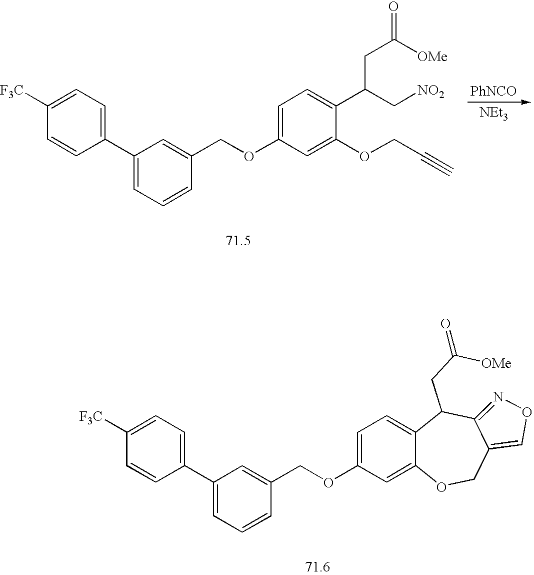

(R/S)-Methyl (7-(((4′-(trifluoromethyl)-1,1′-biphenyl-3-yl)methyl)oxy)-4H,10H-[1]benzoxepino[4,3-c]isoxazol-10-yl)acetate (71.6). A mixture of compound 71.5 (12 mg 0.022 mmol), PhNCO (26 mg, 0.22 mmol) and triethylamine (4 μL) in benzene (5 mL), was stirred at 80° C. for 36 hours. After cooling, the solid was removed from the mixture by filtration, and the filtrate was concentrated and purified by flash chromatography to give 71.6 (8 mg). LC-MS ESI (pos.) m/e: 510.1 (M+H). ¹H NMR (400 MHz) (CDCl₃) δ 8.12(s, 1H), 7.72(s, 4H), 7.67(s, 1H), 7.57(m, 1H), 7.49(m, 2H), 7.24(d, 2H), 6.82(d, 1H), 6.78(dd, 1H), 5.29(d, 1H), 5.13(s, 2H), 4.91(d, 1H), 4.75(dd, 1H), 3.65(s, 3H), 3.18(dd, 1H), 3.10(dd, 1H).

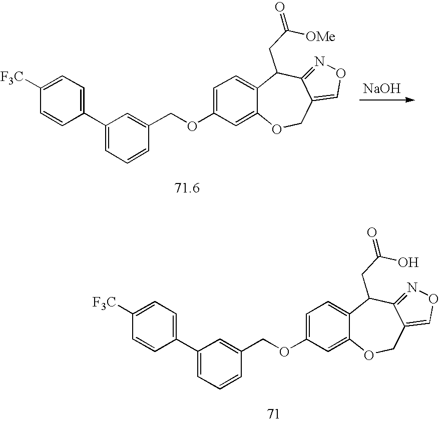

(R/S)-(7-(((4′-(trifluoromethyl)-1,1′-biphenyl-3-yl)methyl)oxy)-4H,10H-[l]benzoxepino[4,3-c]isoxazol-10-yl)acetic acid (71). Compound 71.6 (8 mg, 0.13 mmol) was dissolved in THF (1 mL), MeOH (1 mL) and water (0.5 mL). NaOH (0.2 mL, 2N) was added, and the mixture was stirred at room temperature for 6 hours. The organic solvent was removed under a stream of air. The aqueous layer was acidified by adding 3 N HCl, and was then extracted with DCM. The organic layer was separated, dried with MgSO₄ and concentrated under vacuum. The crude product was purified with flash column to give Example 71 (7 mg). LC-MS ESI (pos.) m/e: 496.1 (M+H). ¹H NMR (400 MHz) (CDCl₃) δ 8.12(s, 1H), 7.71 (s, 4H), 7.66(s, 1H), 7.57(m, 1H), 7.49(m, 2H), 7.25(d, 2H), 6.84(d, 1H), 6.79(dd, 1H), 5.28(d, 1H), 5.13(s, 2H), 4.92(d, 1H), 4.75(dd, 1H), 3.23(dd, 1H), 3.16(dd, 1H).

### Example 72

Example 72 was prepared from compound 29.1 and compound H according to the methods described in Example 29.

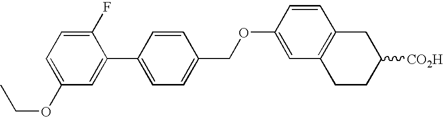

(R/S)-6-(((5′-(ethoxy)-2′-fluoro-1,1′-biphenyl-4-yl)methyl)oxy)-1,2,3,4-tetrahydro-2-naphthalenecarboxylic acid (72) LC-MS ESI (neg.) m/e: 419.0 (M−H).

### Cell-based Aequorin Assay

A cell-based aequorin assay may be employed to characterize the modulatory activity of compounds on the GPR40 signaling pathway. In an exemplary assay, CHO cells are transfected in a 15 cm plated containing 14 million cells with 5 μg of GPR40 expression vector and 5 μg of Aequorin expression vector (Euroscreen) using Lipofectamine 2000 (Invitrogen). After 17-24 hours post-transfection, cells are washed with phosphate buffered saline (PBS) and detached from the tissue culture dish with 2 mL of trypsin (0.25%(w/v)). Trypsinization is halted with 28 mL of Hanks Buffered Salt Solution containing 20 mM Hepes (H/HBSS) and 0.01% fatty acid-free bovine serum albumin (BSA) or 0.625% fatty acid-free human serum albumin (HSA). Coelantrazine is added to 1 ug/mL and the cells are incubated for 2 hours at room temperature. Cells are gently mixed every 15 minutes. Compounds are dissolved in dimethyl sulfoxide for preparation of 10 mM stock solutions. Compounds are diluted in H/HBSS containing either 0.01% BSA or 0.625% HSA. Serial dilutions of the test compounds are prepared to determine dose response.

Aequorin luminescence measurements are made using an EG&G Berthold 96-well luminometer and the response is measured over a 20 second interval after cells and compounds are mixed. The area-under-curve from 2-20 seconds is plotted to determine dose response. The EC₅₀ (effective concentration to reach 50% maximal response) is determined from the dose response plot.

Table 1 includes representative data (EC₅₀ values) obtained for exemplary compounds of the invention for the relative activation of human GPR40. Each of the compounds listed in Table 1 had an EC₅₀ value of less than 10 μM. Therefore, in some embodiments, the invention provides any of the compounds listed in Table 1 individually or as members of a group and pharmaceutically acceptable salts, esters, solvates, tautomers, stereoisomers, and/or prodrugs thereof.

The stereoisomers in Table 1 are as specified, i.e., S-enantiomers or R-enantiomers, and if not specified, or if shown with wavy bonds, are mixtures of S-enantiomers and R-enantiomers. In addition, the present invention provides the S-enantiomers, the R-enantiomers, and mixtures of both S-enantiomers and R-enantiomers including racemates of each compound prepared according to the synthetic methods described herein or adapted with the necessary minor modifications from these methods.

### Insulin Secretion Assay

C57/B16 mice are euthanized with carbon dioxide gas. The pancreatic bile duct is clamped proximal to the duodenum and then cannulated. H/HBSS containing 0.75 mg/mL collagenase XI (Sigma) is then infused into the pancreas through the cannula. The pancreas is excised and then incubated at 37° C. for 13 minutes to complete enzymatic digestion. The collagenase digestion is quenched in H/HBSS containing 1% BSA and washed once in the same buffer. Islets can be purified using density gradient centrifugation using Histopaque (Sigma) and are hand-picked under a stereomicroscope.

Islets are cultured overnight in Roswell Park Memorial Institute (RMPI) media containing 10% fetal bovine serum and 50 uM beta-mercaptoethanol. Following overnight culture, islets are incubated in Dulbecco's Modification of Eagle's medium (DMEM) containing 2.8 mM glucose for one hour.

For determination of insulin secretion, islets are incubated in DMEM containing 12.5 mM glucose and test compounds for one hour. Insulin released into the culture medium from the islets is measured using an insulin ELISA.

TABLE 1
Aequorin Assay Using Human GPR40

| No. | Structure^{a} | Relative EC₅₀ ^{b} |
|---|---|---|
| 1 | 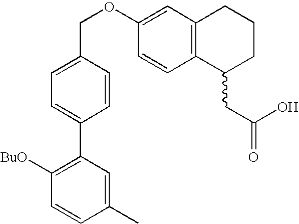 | ++ |
| 2 | 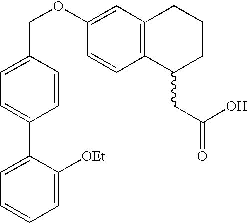 | ++ |
| 3 | 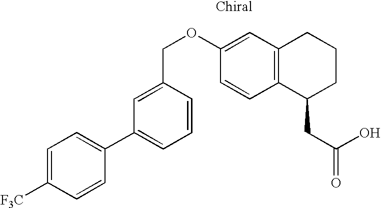 | ++ |
| 4 | 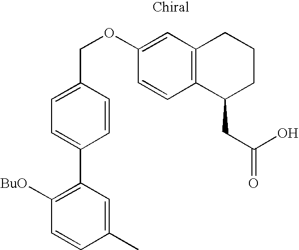 | +++ |
| 5 | 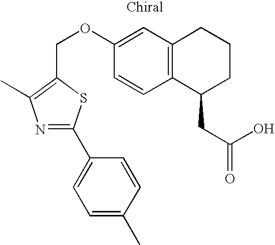 | +++ |
| 6 | 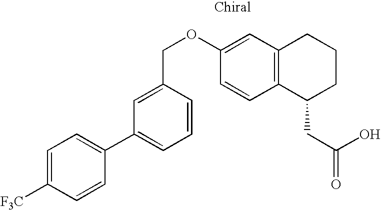 | + |
| 7 | 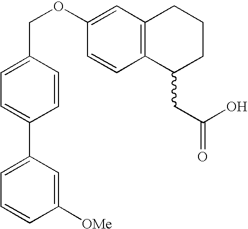 | +++ |
| 8 | 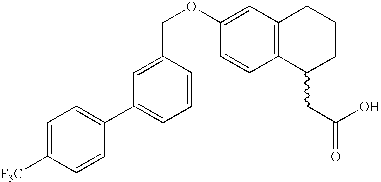 | ++ |
| 9 | 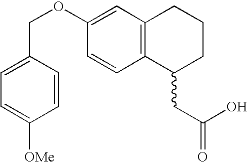 | + |
| 10 | 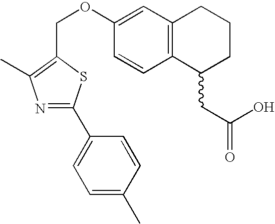 | ++ |
| 11 | 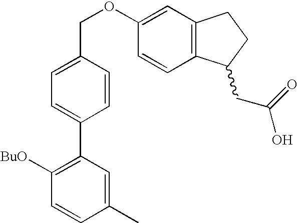 | ++ |
| 12 | 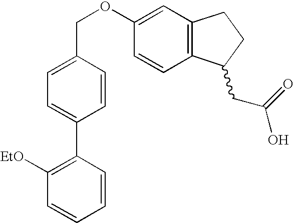 | ++ |
| 13 | 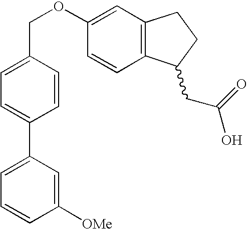 | ++ |
| 14 | 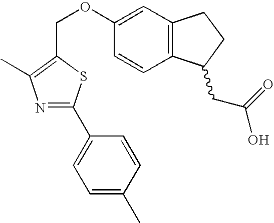 | ++ |
| 15 | 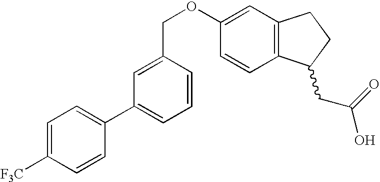 | + |
| 16 | 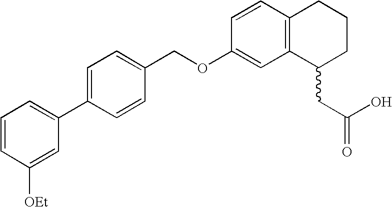 | + |
| 17 | 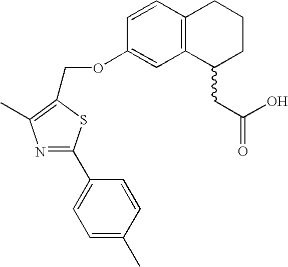 | + |
| 18 | 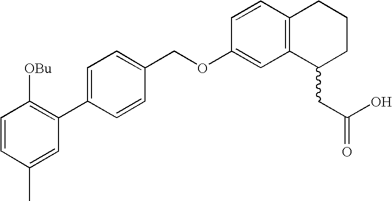 | + |
| 19 | 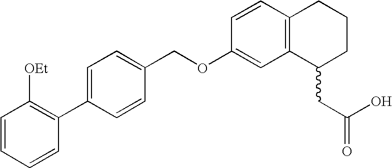 | + |
| 20 | 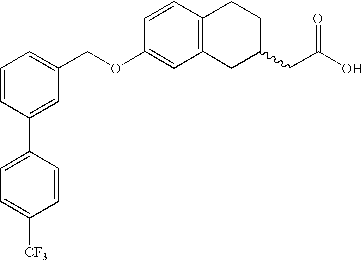 | + |
| 21 | 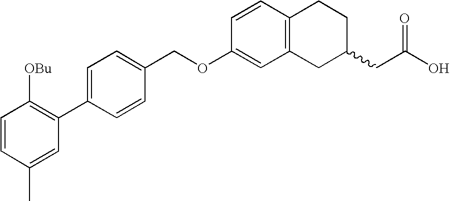 | + |
| 22 | 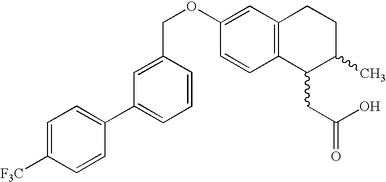 | + |
| 23 | 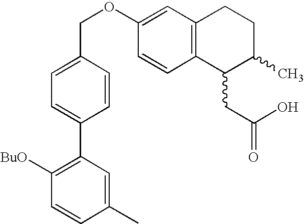 | + |
| 24 | 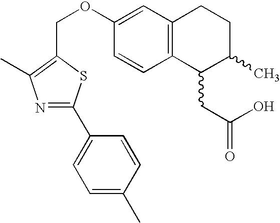 | + |
| 25 | 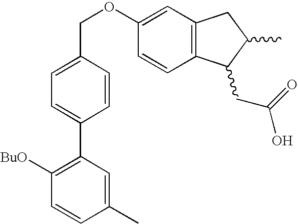 | + |
| 26 | 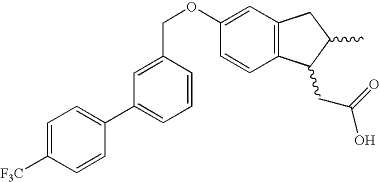 | + |
| 27 | 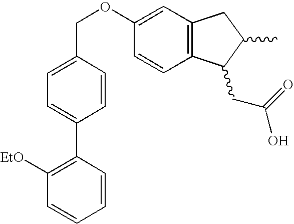 | + |
| 28 | 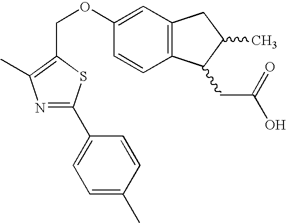 | + |
| 29 | 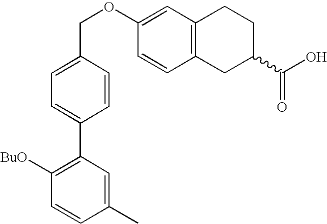 | ++ |
| 30 | 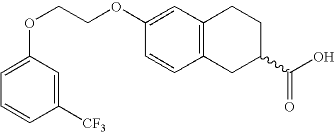 | + |
| 31 | 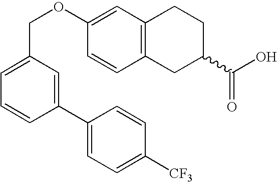 | + |
| 32 | 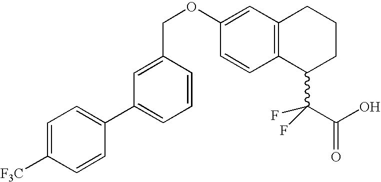 | + |
| 33 | 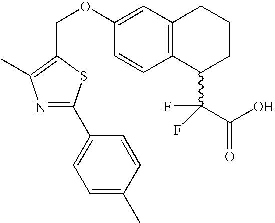 | + |
| 34 | 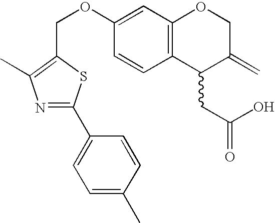 | +++ |
| 35 | 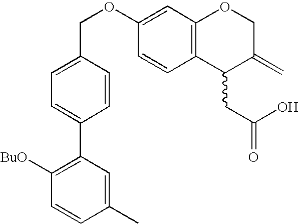 | ++ |
| 36 | 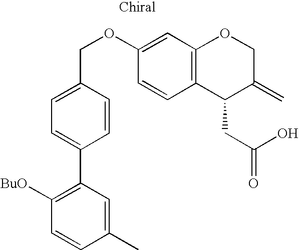 | +++ |
| 37 | 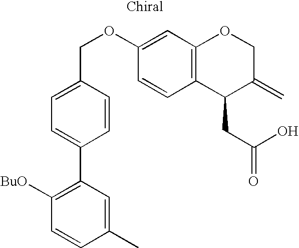 | ++ |
| 38 | 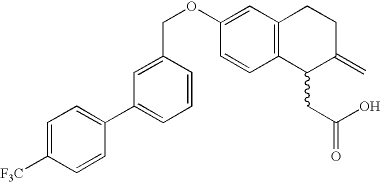 | ++ |
| 39 | 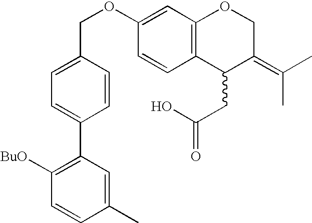 | ++ |
| 40 | 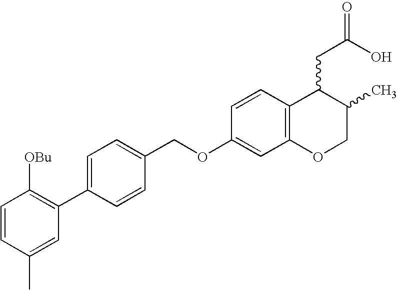 | ++ |
| 41 | 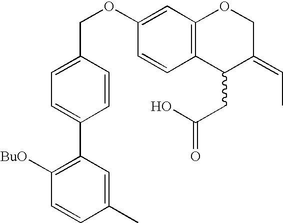 | ++ |
| 42 | 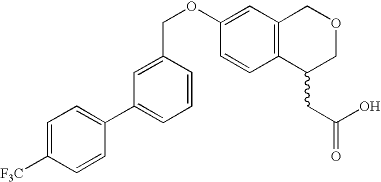 | ++ |
| 43 | 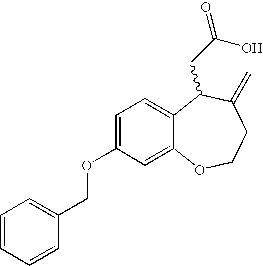 | ++ |
| 44 | 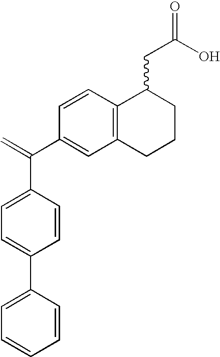 | + |
| 45 | 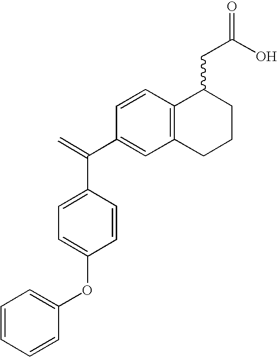 | + |
| 46 | 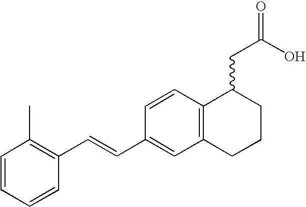 | + |
| 47 | 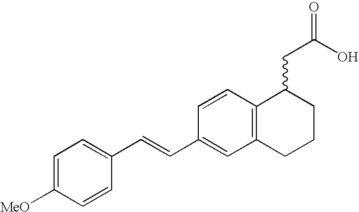 | + |
| 48 | 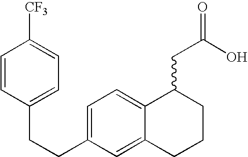 | + |
| 49 | 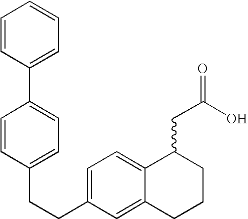 | + |
| 50 | 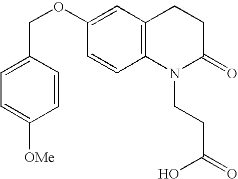 | + |
| 51 | 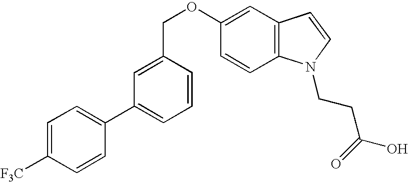 | + |
| 52 | 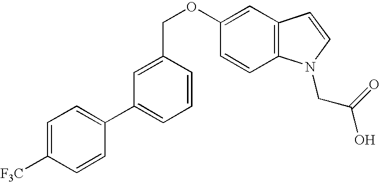 | + |
| 53 | 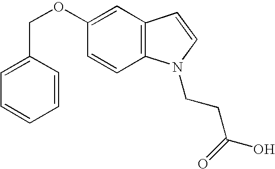 | + |
| 54 | 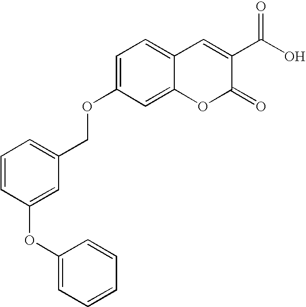 | + |
| 55 | 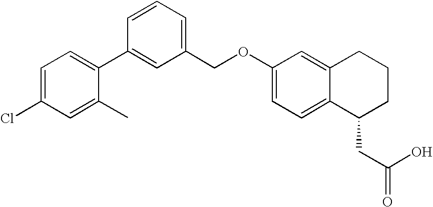 | ++ |
| 56 | 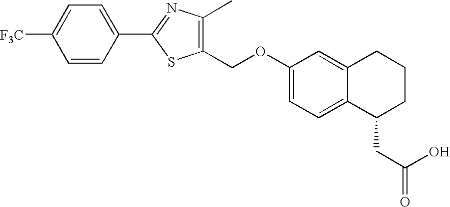 | ++ |
| 57 | 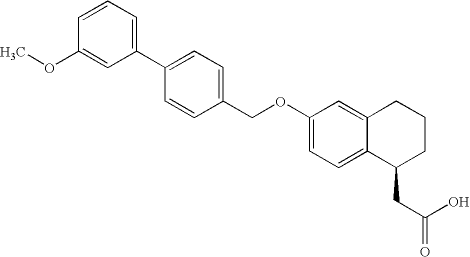 | + |
| 58 | 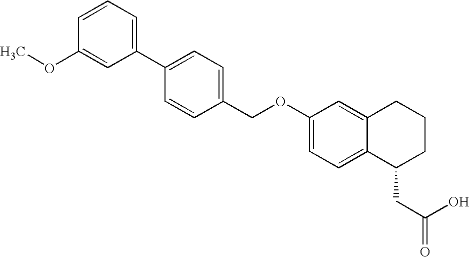 | + |
| 59 | 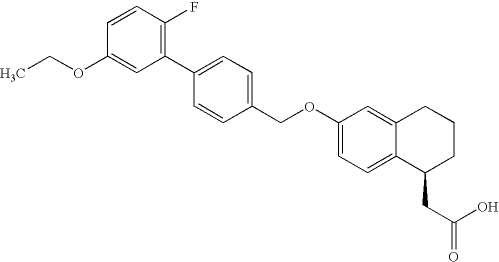 | + |
| 60 | 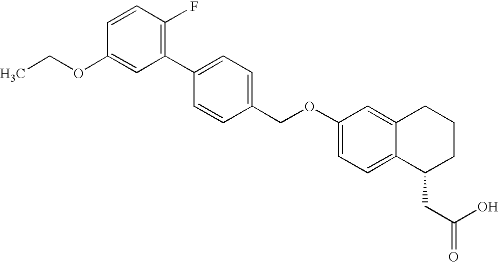 | + |
| 61 | 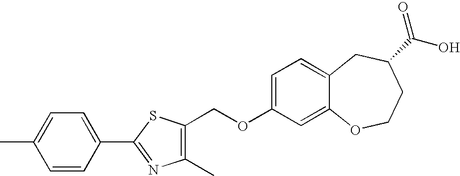 | ++ |
| 63 | 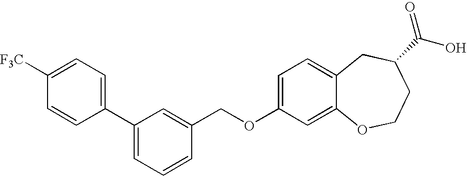 | + |
| 64 | 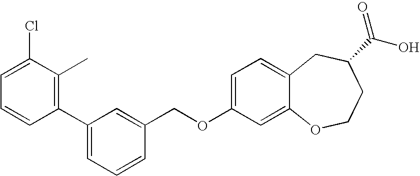 | ++ |
| 65 | 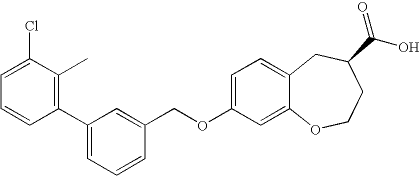 | + |
| 66 | 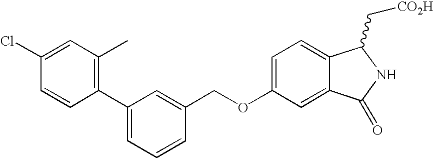 | + |
| 67 | 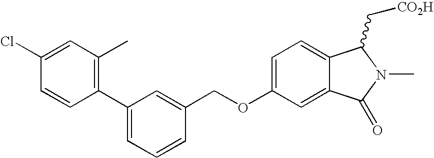 | ++ |
| 68 | 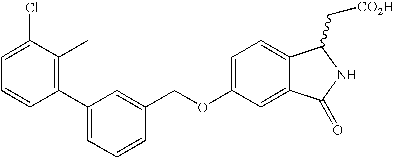 | + |
| 69 | 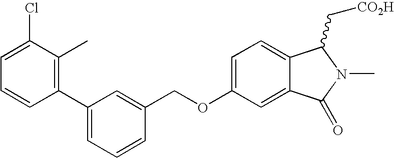 | ++ |
| 70 | 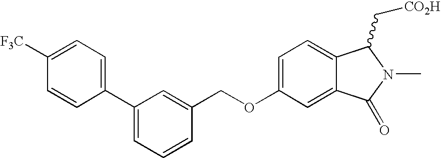 | + |
| 71 | 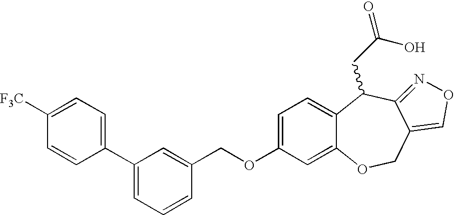 | + |
| 72 | 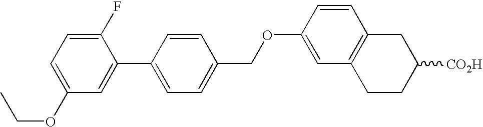 | + |

^{a}When present, the “ 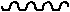 ” bond indicates a mixture of stereoisomers are present in the exemplary compound.
^{b}EC₅₀ Ranges:
+ 1 μM ≦ EC₅₀ < 10 μM
++ 0.1 μM ≦ EC₅₀ < 1 μM
+++ EC₅₀ < 0.1 μM

In some embodiments, the invention provides any one or more of the compounds set forth in the above table either singly or as a member of a group. In some such embodiments, the compound may be a salt of the compound. In other embodiments, the compound may be a racemic mixture or may exist as one of the enantiomers of the compound.

All publications and patent applications cited in this specification are herein incorporated by reference in their entireties and for all purposes as if each individual publication or patent application were specifically and individually indicated as being incorporated by reference and as if each reference was fully set forth in its entirety. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A compound having the formula I: 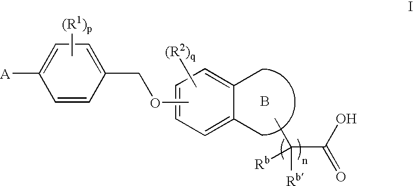
or a pharmaceutically acceptable salt, ester, solvate, tautomer, stereoisomer, or prodrug thereof,
wherein,
A is selected from an aryl group or a heterocyclyl group;
B is a 5 to 7 membered carbocyclic or heterocyclic ring;
R¹ is selected from halo, cyano, C₁-C₆ alkyl, —OH, or C₁-C₆ alkoxy;
R² is selected from halo, C₁-C₆ alkyl, —OH, or C₁-C₆ alkoxy;
n is selected from 0, 1, or 2;
p is selected from 0, 1, or 2;
q is selected from 0, 1, or 2;
each R¹ is independently selected if p is 2; and
each R² is independently selected if q is 2;
R^{b} and R^{b′} are independently selected from —H, or halo;
wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
C₁-C₆ alkoxy,
C₁-C₆ alkyl optionally substituted by halo,
aryl,
halo,
hydroxyl,
heteroaryl,
C₁-C₆ hydroxyalkyl, or
—NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
wherein the B ring may further be substituted with an oxo group or may include a group of formula ═CR^{a}R^{a′} wherein R^{a} and R^{a′} are independently selected from H or C₁-C₄ alkyl groups;
with the proviso that B does not include an O atom if B is a 5-membered ring that comprises four C atoms.

2. The compound of claim 1, wherein n is 1.

3. The compound of claim 1, wherein p is 0.

4. The compound of claim 1, wherein q is 0.

5. The compound of claim 1, wherein A is an optionally substituted aryl group.

6. The compound of claim 5, wherein A is an unsubstituted phenyl group or is a phenyl group that is substituted with at least one cyano, —CF₃, C₁-C₆ alkyl, —OH, or C₁-C₆ alkoxy group.

7. The compound of claim 6, wherein A is a phenyl group substituted with at least one methyl group, methoxy group, ethoxy group, propoxy group, butoxy group, or pentoxy group.

8. The compound of claim 1, wherein B is a 5 or 6 membered carbocyclic or heterocyclic ring.

9. The compound of claim 1, wherein B is a 5 or 6 membered carbocyclic ring.

10. The compound of claim 1, wherein the compound has a formula selected from: 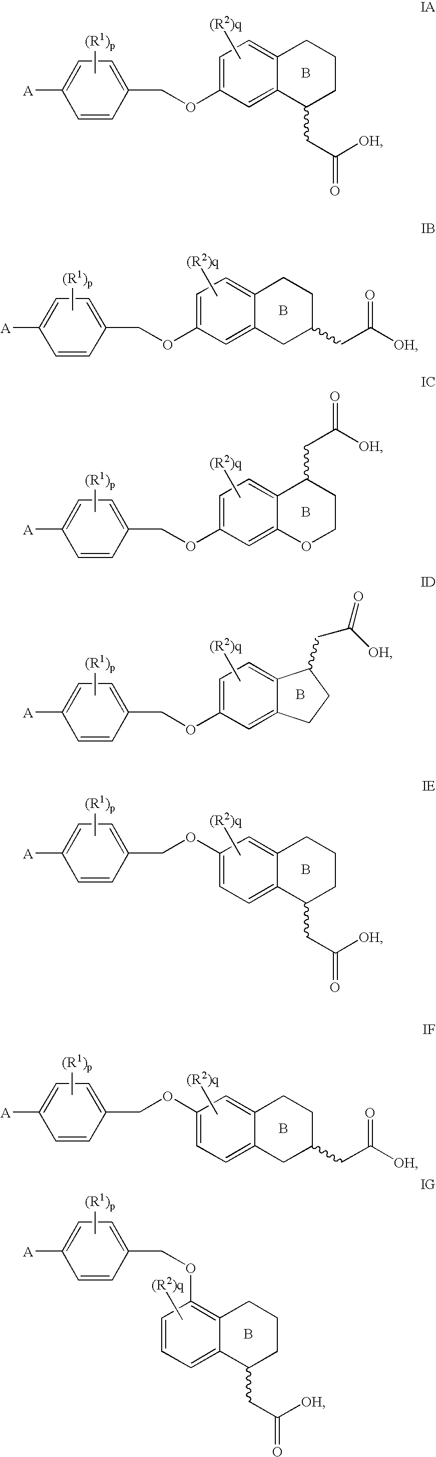 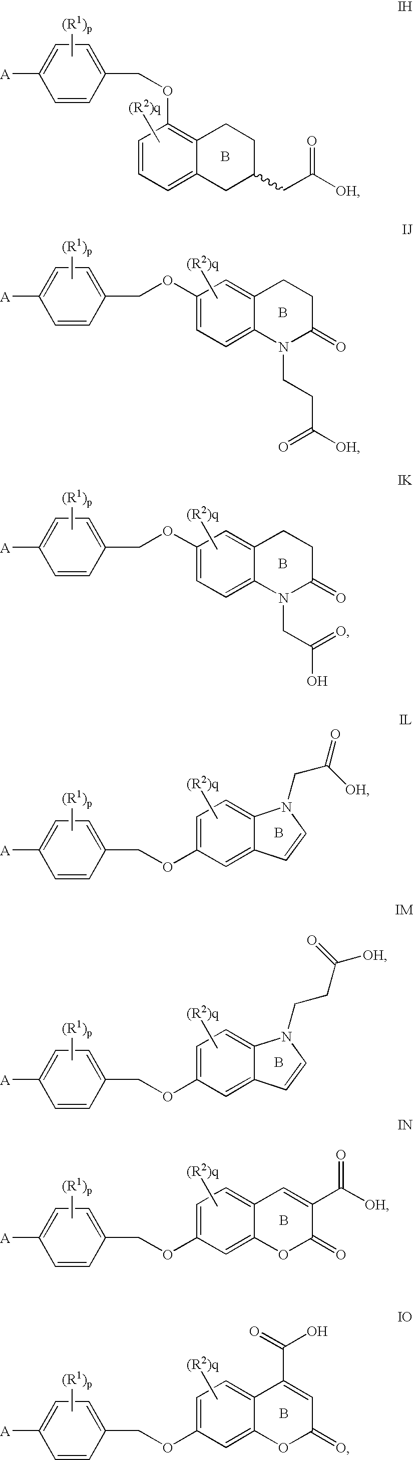 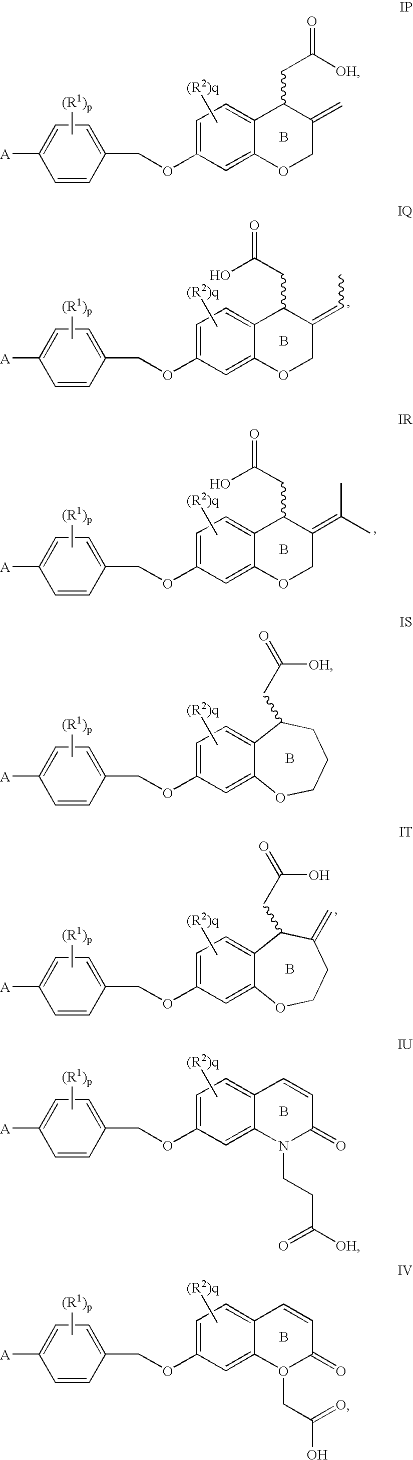 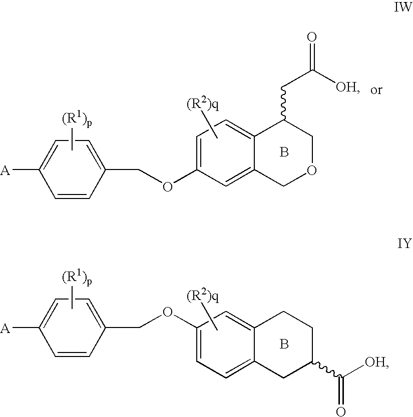
wherein the B ring may be further substituted with a halo, a C₁-C₆ alkyl group, an oxo group, a C₂-C₆ alkenyl group, or a group of formula ═CR^{a}R^{a′} wherein R^{a} and R^{a′} are independently selected from H or C₁-C₄ alkyl groups; and further wherein a wavy bond indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers.

11. The compound of claim 1, wherein the B ring is substituted with a ═CR^{a}R^{a′} group where R^{a} and R^{a′} are independently selected from H and C₁-C₄ alkyl groups.

12. A compound having the formula II: 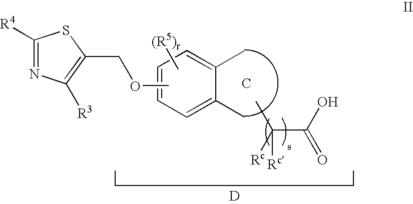
or a pharmaceutically acceptable salt, ester, solvate, tautomer, stereoisomer, or prodrug thereof,
wherein,
C is a 5 to 7 membered carbocyclic or heterocyclic ring;
D is a fragment of the compound as shown above;
R³ is selected from —H, halo, or C₁-C₆ alkyl;
R⁴ is an aryl group;
R⁵ is selected from halo, C₁-C₆ alkyl, —OH, or C₁-C₆ alkoxy;
s is selected from 0, 1, or 2;
r is selected from 0, 1, or 2;
each R⁵ is independently selected if r is 2; and
R^{c} and R^{c′} are independently selected from —H and halo,
wherein each of the above alkyl and aryl groups, and heterocyclic and carbocyclic rings is optionally and independently substituted by 1 to 3 substituents selected from
amino,
aryl, heteroaryl, cycloalkyl, or heterocyclyl optionally substituted by 1-5
substituents selected from
C₁-C₆ alkoxy,
C₁-C₆ alkyl optionally substituted by halo,
aryl,
halo,
hydroxyl,
heteroaryl,
C₁-C₆ hydroxyalkyl, or
—NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
and further wherein the C ring may further be substituted with an oxo group or may include a group of formula ═CR^{a}R^{a′} wherein R^{a} and R^{a′} are independently selected from H or C₁-C₄ alkyl groups.

13. The compound of claim 12, wherein s is 1.

14. The compound of claim 12, wherein r is 0.

15. The compound of claim 12, wherein R⁴ is an unsubstituted phenyl group or is a phenyl group that is substituted with at least one cyano, halo, —CF₃, C₁-C₆ alkyl, —OH, or C₁-C₆ alkoxy group.

16. The compound of claim 15, wherein R⁴ is a phenyl group substituted with a methyl group.

17. The compound of claim 15, wherein R⁴ is a phenyl group substituted in the para position with a methyl group.

18. The compound of claim 12, wherein R³ is a C₁-C₆ alkyl group.

19. The compound of claim 18, wherein R³ is a methyl, ethyl, or propyl group.

20. The compound of claim 18, wherein R³ is a methyl group.

21. The compound of claim 12, wherein C is a 5 or 6 membered carbocyclic or heterocyclic ring.

22. The compound of claim 12, wherein C is a 5 or 6 membered carbocyclic ring.

23. The compound of claim 12, wherein fragment D has a formula selected from: 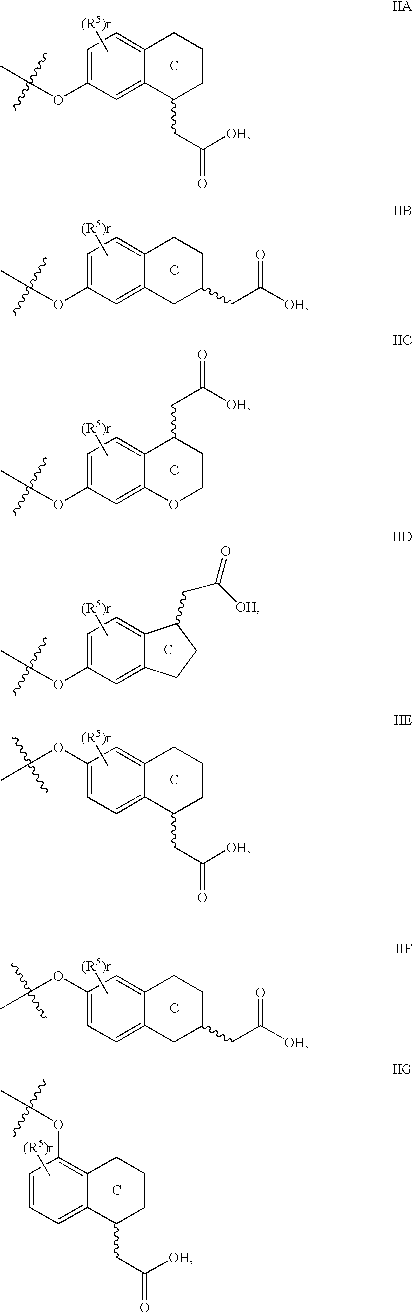 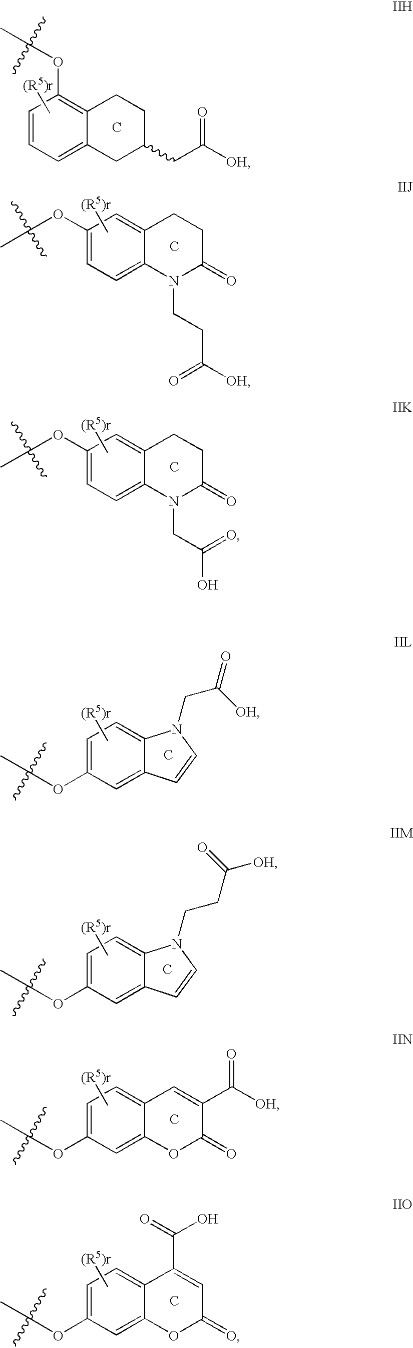 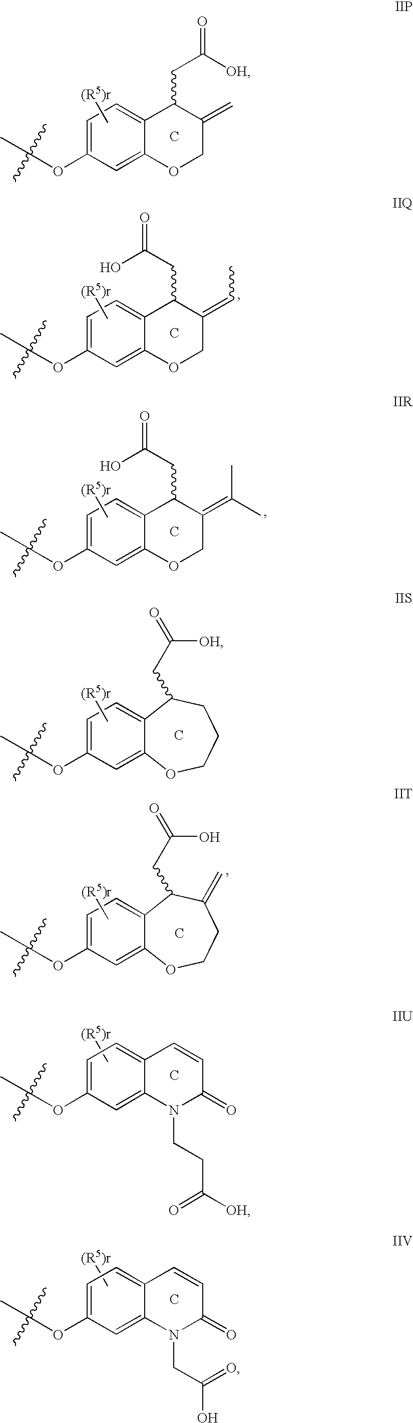 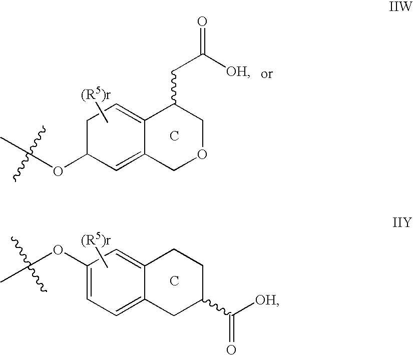
wherein the C ring may be further substituted with a halo, a C₁-C₆ alkyl group, an oxo group, a C₂-C₆ alkenyl group, or a group of formula ═CR^{a}R^{a′} wherein R^{a} and R^{a′} are independently selected from H or C₁-C₄ alkyl groups; and further wherein a wavy bond indicates a point of attachment when drawn across a bond, indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers.

24. The compound of claim 12, wherein the C ring is substituted with a ═CR^{a}R^{a′} group where R^{a} and R^{a′} are independently selected from H and C₁-C₄ alkyl groups.

25. A compound having the formula III:
F-L₁-E-L₂-L₃-G III
or a pharmaceutically acceptable salt, ester, solvate, tautomer, stereoisomer, or prodrug thereof,
wherein,
E is selected from an aryl group or a heterocyclyl group;
F is selected from —H, an aryl group, or a heterocyclyl group;
L₁ is selected from a bond, —O—, —NH—, —S—, —CH₂—, —C(═O)—, —SO—, or —SO₂—;
L₂ is selected from —(CH₂)ₘ—, or —O—(CH₂)ₘ— where m is selected from 1 or 2;
L₃ is —O—, —NH—, —S—, or L₂ and L₃, when taken together, represent a group of formula —CH═CH—, or —C(═CH₂)—; and
G is selected from 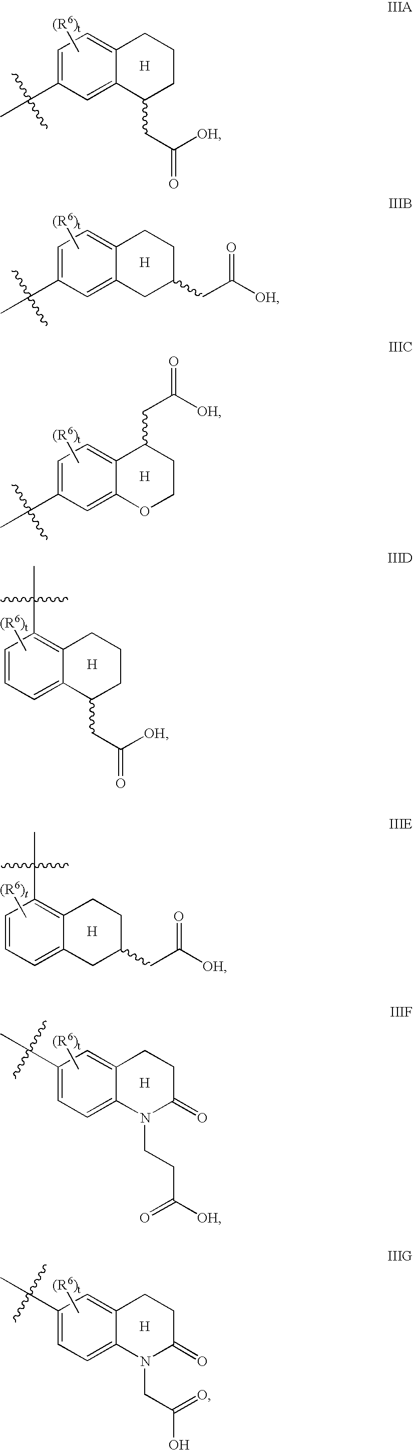 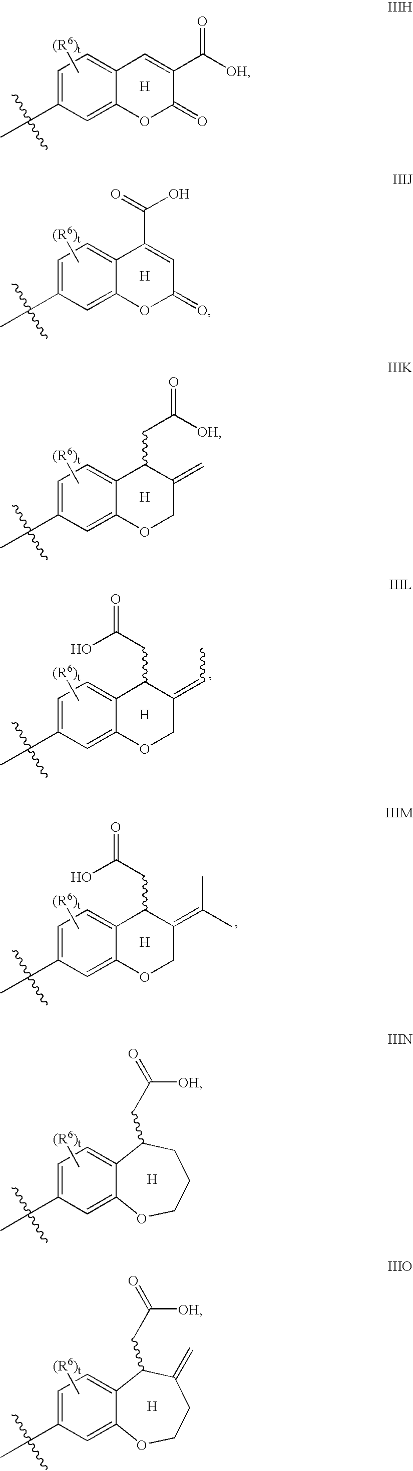 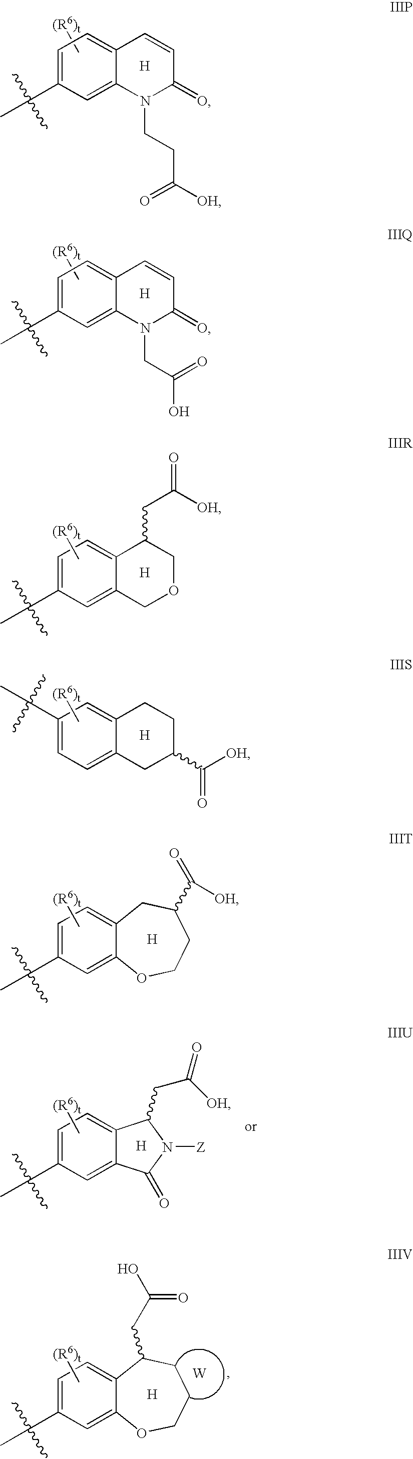
wherein,
R⁶ is selected from halo, C₁-C₆ alkyl, —OH, or C₁-C₆ alkoxy;
t is selected from 0, 1, or 2;
each R⁶ is independently selected if t is 2;
Z is selected from H and C₁-C₆ alkyl; and
W is a heterocyclic ring;
and further wherein the H ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups, and a wavy bond indicates a point of attachment when drawn across a bond, indicates the R and S enantiomers individually or as a mixture of the R and S enantiomers, and, when the wavy bond is attached to a carbon that is double bonded to another carbon atom, indicates the cis and trans isomers individually or as a mixture of the cis and trans isomers;
wherein if G is IIIT, L³ is —O—, L² is —(CH₂)—, L¹ is a bond, E is an unsubstituted benzene ring, and F and L² are oriented in a meta substitution pattern on E, then F is not substituted with two methyl groups, wherein if G is IIIT, L³ is —O—, L² is —(CH₂)—, L¹ is —O—, E is an unsubstituted benzene ring, and L¹ and L² are oriented in a meta substitution pattern on E, then F is not an unsubstituted benzene ring,
and further wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
C₁-C₆ alkoxy,
C₁-C₆ alkyl optionally substituted by halo,
aryl,
halo,
hydroxyl,
heteroaryl,
C₁-C₆ hydroxyalkyl, or
—NHS(O)₂—(C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl.

26. The compound of claim 25, wherein L₁ is a bond or —O—.

27. The compound of claim 25, wherein L₃ is —O—.

28. The compound of claim 25, wherein L₂ is —CH₂)ₘ— and m is 1.

29. The compound of claim 25, wherein E is an optionally substituted thiazole group.

30. The compound of claim 29, wherein the compound of formula III is a compound of formula IV 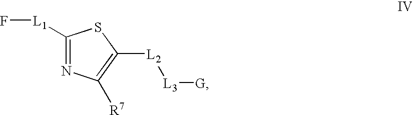
wherein R⁷ is selected from —H, halo, or C₁-C₆ alkyl.

31. The compound of claim 30, wherein R⁷ is a methyl group.

32. The compound of claims 25, wherein E is an optionally substituted phenyl group.

33. The compound of claim 32, wherein the compound of formula III is a compound of formula VA or VB 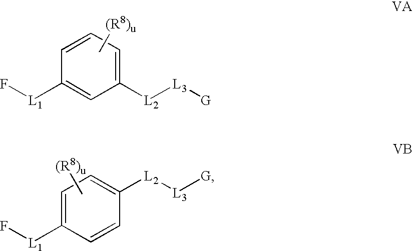
wherein,
R⁸ is selected from halo, cyano, C₁-C₆ alkyl, —OH, or C₁-C₆ alkoxy;
u is selected from 0, 1, or 2; and
each R⁸ is independently selected if u is 2.

34. The compound of claim 25, wherein F is an unsubstituted phenyl group or is a phenyl group that is substituted with at least one cyano, —CF₃, C₁-C₆ alkyl, —OH, or C₁-C₆ alkoxy group.

35. The compound of claim 34, wherein F is a phenyl group substituted with at least one methyl group, methoxy group, ethoxy group, propoxy group, butoxy group, or pentoxy group.

36. The compound of claim 25, wherein the H ring is substituted with a ═CR^{a}R^{a′} group where R^{a} and R^{a′} are independently selected from H and C₁-C₄ alkyl groups.

37. A compound having the formula VI: 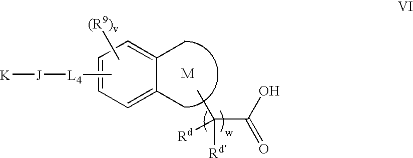
or a pharmaceutically acceptable salt, ester, solvate, tautomer, stereoisomer, or prodrug thereof,
wherein,
J is selected from an aryl group or a heterocyclyl group;
K is selected from —H, —CF₃, halo, cyano, C₁-C₆ alkyl, —OH, C₁-C₆ alkoxy, —O-aryl, an aryl group, or a heterocyclyl group;
M is a 5 to 7 membered carbocyclic or heterocyclic ring;
L₄ is selected from —CH₂CH₂—, —CH═CH—, or —C(═CH₂)—;
R⁹ is selected from halo, C₁-C₆ alkyl, —OH, or C₁-C₆ alkoxy;
v is selected from 0, 1, or 2;
w is selected from 0, 1, or 2;
each R⁹ is independently selected if v is 2; and
R^{d} and R^{d′} are independently selected from —H and halo,
and further wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
C₁-C₆ alkoxy,
C₁-C₆ alkyl optionally substituted by halo,
aryl,
halo,
hydroxyl,
heteroaryl,
C₁-C₆ hydroxyalkyl, or
—NHS(O)₂—(C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
and further wherein the M ring may be further substituted with an oxo group or a group of formula ═CR^{a}R^{a′} where R^{a} and R^{a′} are independently selected from H or C₁-C₄ alkyl groups.

38. The compound of claim 37, wherein w is 1.

39. The compound of claim 37, wherein v is 0.

40. The compound of claim 37, wherein J is an optionally substituted aryl group.

41. The compound of claim 37, wherein J is an optionally substituted thiazole group.

42. The compound of claim 37, wherein M is a 6 membered carbocyclic or heterocyclic ring.

43. The compound of claim 42, wherein M is a 6 membered carbocyclic ring.

44. The compound of claim 37, wherein the M ring is substituted with a ═CR^{a}R^{a′} group where R^{a} and R^{a′} are independently selected from H and C₁-C₄ alkyl groups.

45. A compound having the formula VII: 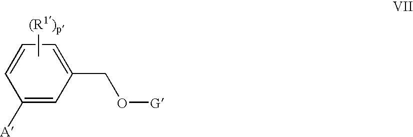
or a pharmaceutically acceptable salt, ester, solvate, tautomer, stereoisomer, or prodrug thereof,
wherein,
A′ is selected from an aryl group or a heterocyclyl group;
R^{1′} is selected from halo, cyano, C₁-C₆ alkyl, —OH, or C₁-C₆ alkoxy;
p′ is selected from 0, 1, or 2;
each R^{1′} is independently selected if p is 2; and
G′ is selected from 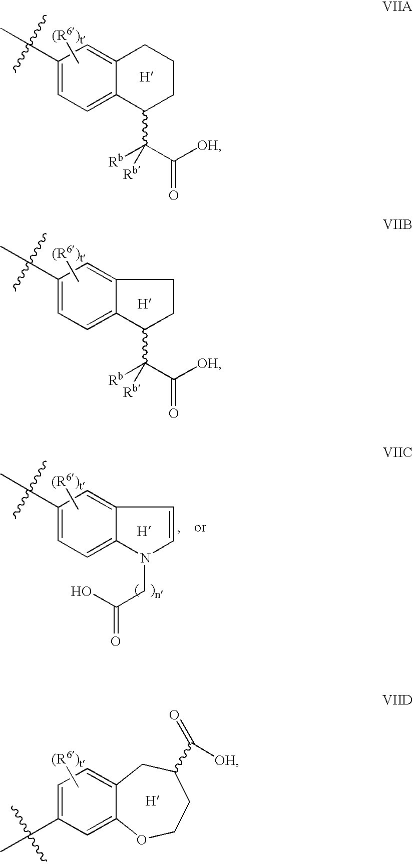
wherein,
R^{6′} is selected from halo, C₁-C₆ alkyl, —OH, or C₁-C₆ alkoxy;
t′ is selected from 0, 1, or 2;
each R^{6′} is independently selected if t′ is 2;
R^{b} and R^{b′} are independently selected from —H and halo; and
n′ is selected from 1 or 2
and further wherein the H′ ring may be further substituted with a halo, a C₁-C₆ alkyl group, an oxo group, a C₂-C₆ alkenyl group, or a group of formula ═CR^{a}R^{a′} where R^{a} and R^{a′} are independently selected from H or C₁-C₄ alkyl groups, and a wavy bond indicates a point of attachment when drawn across a bond, or indicates the R and S enantiomers individually or as a mixture of the R and S enantiomers;
and further wherein each of the above alkyl, aryl, and heterocyclyl groups, and heterocyclic and carbocyclic rings is optionally and independently substituted by 1 to 3 substituents selected from
amino,
aryl, heteroaryl, cycloalkyl, or heterocyclyl optionally substituted by 1-5 substituents selected from
C₁-C₆ alkoxy,
C₁-C₆ alkyl optionally substituted by halo,
aryl,
halo,
hydroxyl,
heteroaryl,
C₁-C₆ hydroxyalkyl, or
—NHS(O)₂—C₁-C₆ alkyl);
C₁-C₆ alkyl, C₁-C₆ haloalkyl, C₁-C₆ hydroxyalkyl, C₁-C₆ alkoxy, C₁-C₆ alkylamino, C₂-C₆ alkenyl, or C₂-C₆ alkynyl, wherein each of which may be interrupted by one or more heteroatoms,
cyano,
halo,
hydroxyl,
nitro, or
—O-aryl,
and further wherein, A′ does not have the following formula

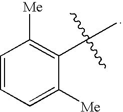

46. The compound of claim 45, wherein A′ is a phenyl group that is substituted with at least one cyano, —CF₃, C₁-C₆ alkyl, —OH, or C₁-C₆ alkoxy group.

47. The compound of claim 45, wherein A′ is a phenyl group that is substituted with at least one —CF₃, —F, —Cl, —Br, —I, methoxy group, ethoxy group, propoxy group, butoxy group, or pentoxy group.

48. The compound of claim 45, wherein p′ is 0.

49. The compound of claim 45, wherein t′ is 0.

50. The compound of claim 45, wherein G′ is VIIA.

51. The compound of claim 45, wherein G′ is VIIB.

52. The compound of claim 45, wherein G′ is VIIC.

53. The compound of claim 45, wherein G′ is VIID.

54. The compound of claim 45, wherein H′ is not further substituted.

55. The compound of claim 45, wherein H′ is substituted with a C₁-C₄ alkyl group.

56. The compound of claim 45, wherein H′ is substituted with a group of formula ═CR^{a}R^{a′} where R^{a} and R^{a′} are independently selected from H or C₁-C₄ alkyl groups.

57. A pharmaceutical composition, comprising: a pharmaceutically acceptable carrier, diluent or excipient and the compound, pharmaceutically acceptable salt, ester, solvate, tautomer, stereoisomer, or prodrug of any one of claims 1, 12, 25, 37, or 45.

58. A method for treating a disease or condition selected from type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer or edema, comprising: administering to a subject in need thereof a therapeutically effective amount of the compound, pharmaceutically acceptable salt, ester, solvate, tautomer, stereoisomer, or prodrug of any one of claims 1, 12, 25, 37, or 45.

59. The method of claim 58, wherein the disease or condition is type II diabetes.

60. The method of claim 58, wherein the compound, pharmaceutically acceptable salt, ester, solvate, tautomer, stereoisomer, or prodrug or pharmaceutical composition is administered in combination with a second therapeutic agent.

61. A method for modulating circulating insulin concentration in a subject, comprising: administering the compound, pharmaceutically acceptable salt, ester, solvate, tautomer, stereoisomer, or prodrug of any one of claims 1, 12, 25, 37, or 45 to the subject.
